(19) United States
(12) Patent Application Publication
(10) Pub. No.: US 2026/0285912 A1
(43) Pub. Date: Sep. 24, 2026

(54) MODULAR RE-TARGETING OF RHABDOVIRAL (G) GLYCOPROTEINS FROM THEIR NATURAL RECEPTORS

(71) Applicant: Vyriad, Rochester, MN (US)

(72) Inventors: Stephen J. RUSSELL, Rochester, MN (US); Gopal Naik NENAVATH, Rochester, MN (US)

(73) Assignee: Vyriad, Rochester, MN (US)

(21) Appl. No.: 19/474,285

(22) PCT Filed: Apr. 12, 2024
(86) PCT No.: PCT/US2024/024511

§ 371 Date: Oct. 10, 2025

Related U.S. Application Data
(60) Provisional application No. 63/495,702, filed on Apr. 12, 2023.
(60) Provisional application No. 63/502,585, filed on May 16, 2023.
(60) Provisional application No. 63/586,976, filed on Sep. 29, 2023.
(60) Provisional application No. 63/625,842, filed on Jan. 26, 2024.
(60) Provisional application No. 63/626,979, filed on Jan. 30, 2024.
(60) Provisional application No. 63/560,911, filed on Mar. 4, 2024.
(60) Provisional application No. 63/633,553, filed on Apr. 12, 2024.

(51) Int. Cl. C07K 14/005; C07K 14/485; C07K 16/28; C07K 16/32; C12N 15/86

(52) U.S. Cl. CPC C07K 14/005; C07K 14/485; C07K 16/2809; C07K 16/2863; C07K 16/32; C12N 15/86; C07K 2317/622; C07K 2319/30; C07K 2319/70; C12N 2740/15043; C12N 2740/15052; C12N 2760/20222

(57) ABSTRACT

In aspects, the present disclosure provides a recombinant fusion protein comprising, consisting essentially of, or consisting of (a) a rhabdoviral G glycoprotein or a functional fragment or derivative thereof and (b) a polypeptide (polypeptide tag) that is capable of forming a covalent bond with an amino acid residue in a second recombinant fusion protein, wherein the polypeptide tag is N-terminal to the rhabdoviral G glycoprotein, and wherein the second recombinant fusion protein comprises a polypeptide (polypeptide catcher) that is capable of forming a covalent bond with an amino acid residue in the polypeptide tag. Additional aspects are as described herein.

### CROSS-REFERENCE TO RELATED APPLICATIONS

This application claims the priority benefit of U.S. Provisional Patent Application No. 63/495,702, filed Apr. 12, 2023; U.S. Provisional Patent Application No. 63/502,585, filed May 16, 2023; U.S. Provisional Patent Application No. 63/586,976, filed Sep. 29, 2023; U.S. Provisional Patent Application No. 63/625,842, filed Jan. 26, 2024; U.S. Provisional Patent Application No. 63/626,979, filed Jan. 30, 2024; U.S. Provisional Patent Application No. 63/560,911, filed Mar. 4, 2024; and U.S. Provisional Patent Application No. 63/633,553, filed Apr. 12, 2024; the disclosures of each of which are incorporated herein by reference in their entireties.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 832,607 Byte XML (Extensible Markup Language) file named “770398.xml” created on Apr. 12, 2024.

### BACKGROUND

There is an ongoing need in the art for components that can be used in a virus or enveloped delivery vehicle to target the virus or enveloped delivery vehicle to particular cells. There is also an ongoing need in the art for viruses and enveloped delivery vehicles that incorporate such components.

### BRIEF SUMMARY

In aspects, the present disclosure provides a recombinant fusion protein comprising, consisting essentially of, or consisting of (a) a rhabdoviral G glycoprotein or a functional fragment or derivative thereof and (b) a polypeptide (polypeptide tag) that is capable of forming a covalent bond with an amino acid residue in a second recombinant fusion protein, wherein the polypeptide tag is N-terminal to the rhabdoviral G glycoprotein, and wherein the second recombinant fusion protein comprises a polypeptide (polypeptide catcher) that is capable of forming a covalent bond with an amino acid residue in the polypeptide tag.

In aspects, the present disclosure provides a recombinant protein system comprising, consisting essentially of, or consisting of (a) a recombinant fusion protein comprising, consisting essentially of, or consisting of (1) a rhabdoviral G glycoprotein or a functional fragment or derivative thereof and (2) a polypeptide (polypeptide tag) that is capable of forming a covalent bond with an amino acid residue in a second recombinant fusion protein, wherein the polypeptide tag is N-terminal to the rhabdoviral G glycoprotein, and (b) the second recombinant fusion protein, wherein the second recombinant fusion protein comprises a polypeptide (polypeptide catcher) that is capable of forming a covalent bond with an amino acid residue in the polypeptide tag.

In aspects, the present disclosure provides a membraned vesicle comprising, consisting essentially of, or consisting of a recombinant fusion protein as described herein or a recombinant protein system as described herein.

In aspects, the present disclosure provides an enveloped viral particle comprising, consisting essentially of, or consisting of a recombinant fusion protein as described herein or a recombinant protein system as described herein.

In aspects, the present disclosure provides a recombinant viral vector comprising, consisting essentially of, or consisting of a nucleotide encapsulated by a membraned vesicle as described herein or an enveloped viral particle as described herein.

In aspects, the present disclosure provides a composition comprising, consisting essentially of, or consisting of a pharmaceutically acceptable carrier and a membraned vesicle as described herein, an enveloped viral particle as described herein, or a recombinant viral vector as described herein.

In aspects, the present disclosure provides a method of delivering a payload to a target cell, the method comprising, consisting essentially of, or consisting of contacting the target cell with a membraned vesicle as described herein, an enveloped viral particle as described herein, a recombinant viral vector as described herein, or a composition as described herein.

In aspects, the present disclosure provides a retroviral vector expression system comprising, consisting essentially of, or consisting of one or more nucleotide sequences encoding a recombinant fusion protein as described herein or a recombinant protein system as described herein.

In aspects, the present disclosure provides a method of making a membraned vesicle, an enveloped viral particle, or a recombinant viral vector, the method comprising, consisting essentially of, or consisting of: a) transfecting or transducing a packaging host cell with a retroviral vector expression system as described herein; and b) recovering the membraned vesicle, enveloped viral particle, or recombinant viral vector produced by the transfected or transduced packaging host cell.

In aspects, the present disclosure provides a method of making a membraned vesicle, an enveloped viral particle, or a recombinant viral vector, the method comprising, consisting essentially of, or consisting of: a) transfecting or transducing a first host cell with a first vector expression system comprising, consisting essentially of, or consisting of one or more nucleotide sequences encoding a recombinant fusion protein as described herein, wherein the first host cell is a packaging host cell; b) transfecting or transducing a second host cell with a second vector expression system comprising, consisting essentially of, or consisting of one or more nucleotide sequences encoding the second recombinant fusion protein; c) collecting supernatant from the transfected or transduced second host cell; d) administering the supernatant to the transfected or transduced first host cell; and e) recovering the membraned vesicle, enveloped viral particle, or recombinant viral vector produced by the transfected or transduced first host cell after administration of supernatant produced by the transfected or transduced second host cell.

In aspects, the present disclosure provides a plasmid comprising, consisting essentially of, or consisting of one or more nucleotide sequences encoding a recombinant fusion protein as described herein or a recombinant protein system as described herein.

In aspects, the present disclosure provides a composition as described herein or a retroviral vector expression system as described herein for use in treating a disease in a mammal.

In aspects, the present disclosure provides a method of making a mixed rhabdoviral G glycoprotein trimer, the method comprising, consisting essentially of, or consisting of: a) transfecting or transducing a packaging host cell with a retroviral vector expression system as described herein; and b) recovering the mixed rhabdoviral G glycoprotein trimer.

In aspects, the present disclosure provides a method of making a mixed rhabdoviral G glycoprotein trimer, the method comprising, consisting essentially of, or consisting of: a) transfecting or transducing a first host cell with a first vector expression system comprising, consisting essentially of, or consisting of one or more nucleotide sequences encoding a recombinant fusion protein as described herein, wherein the first host cell is a packaging host cell; b) transfecting or transducing a second host cell with a second vector expression system comprising, consisting essentially of, or consisting of one or more nucleotide sequences encoding the second recombinant fusion protein; c) contacting virus produced by the first host cell with second recombinant fusion protein produced by the second host cell; and d) obtaining virus decorated with the second recombinant fusion protein, wherein the virus comprises a mixed trimer, wherein the mixed trimer comprises (a) at least one recombinant fusion protein, wherein the polypeptide tag of the recombinant fusion protein is covalently bound to the polypeptide catcher of the second fusion protein, and (b) at least one (i) recombinant fusion protein, wherein the polypeptide tag of the recombinant fusion protein is not covalently bound to a polypeptide catcher or (ii) rhabdoviral G glycoprotein or a functional fragment or derivative thereof that is not within a fusion protein.

In aspects, the present disclosure provides a method of reducing inhibition of rhabdoviral G glycoprotein-mediated fusion in serum, wherein the inhibition is by complement inactivation or competitive binding with low-density lipoprotein (LDL) or very-low-density lipoprotein (vLDL), the method comprising, consisting essentially of, or consisting of covalently bonding to a rhabdoviral G glycoprotein or functional fragment or derivative thereof a targeting molecule, and exposing the rhabdoviral G glycoprotein or functional fragment or derivative thereof with targeting molecule to serum, wherein complement inactivation or competitive binding with LDL or vLDL is inhibited.

In aspects, the present disclosure provides a method of reducing inhibition of rhabdoviral G glycoprotein-mediated fusion in serum, wherein the inhibition is by complement inactivation or competitive binding with LDL or vLDL, the method comprising, consisting essentially of, or consisting of producing a rhabdoviral G glycoprotein or functional fragment or derivative thereof as the rhabdoviral G glycoprotein or functional fragment or derivative thereof within a recombinant fusion protein as described herein, and exposing the rhabdoviral G glycoprotein or functional fragment or derivative thereof to serum, wherein complement inactivation or competitive binding with LDL or vLDL is inhibited.

Additional aspects are as described herein.

Without wishing to be bound by any particular theory, there may be discussion herein of beliefs or understandings of underlying principles relating to the materials and methods disclosed herein. It is recognized that regardless of the ultimate correctness of any mechanistic explanation or hypothesis, an aspect of the disclosure can nonetheless be operative and useful

### BRIEF DESCRIPTION OF THE DRAWINGS

FIG. 1A shows the Vesiculovirus indiana G glycoprotein (VSIV-G) amino acid sequence (SEQ ID NO: 21) with SpyTag (ST) insertion sites bolded and labeled. Throughout the figures the abbreviation VSV refers to Vesiculovirus indiana.

FIG. 1B is a drawing of the VSIV-G crystal structure with SpyTag (ST) insertion sites labeled.

FIG. 2 is a western blot showing the binding of a VSIV antibody (anti-VSV-G) to pCG-4MC11-G-WT constructs with different SpyTag insertion sites either without (left) or with (right) flanking linkers.

FIGS. 3A-3C are flow cytometry histograms showing cell surface localization of VSIV-G SpyTag constructs with or without linkers at insertion sites 1-4 (FIG. 3A) or insertion sites 7, 9, and 11 (FIG. 3B), and a bar graph quantification showing a comparison of all the constructs (FIG. 3C). The SpyTag insertion site is indicated by ST #, the presence of a linker is indicated by -L, wild type VSIV-G (listed as VSV-WT-G, G-WT, or WT-G in the figures) was used as a positive control, and a negative control was also used (Mock). In FIG. 3C the leftmost bar shows the wild type VSIV-G (WT-G) Median Fluorescence Intensity (MFI) and the MFI for the SpyTag VSIV-G constructs with different insertion sites (ST #1, ST #2, ST #3, ST #4, ST #7, ST #9, ST #11) is shown for each construct without linker (ST, left bar) and with linker (ST w/Linker, right bar).

FIGS. 3D-3E are a flow cytometry histogram showing cell surface localization of VSV-G constructs with wild type G (G-WT) (SEQ ID NO: 21), G with deletions at the K47, R354, and Y209 residues (G-delKYR) (SEQ ID NO: 27), G with Q substitutions at the K47, R354, and Y209 residues (SEQ ID NO: 26) and a SpyTag insertion (SEQ ID NO: 1) with linker (SEQ ID NO: 12) at site 1 (G-QQQ-ST #1-L), and a negative control (Mock) (FIG. 3D), and a bar graph quantification showing a comparison of all the constructs (FIG. 3E).

FIGS. 4A-4B are graphs showing the results of a dual split protein (DSP) assay for SpyTag-VSV-G pseudotyped lentiviruses with SpyTag insertion at sites 1, 2, or 3 (FIG. 4A) and at sites 4, 7, 9, or 11 (FIG. 4B), with (+Linker) and without (-Linker) linkers, in wild type G (G-WT) (SEQ ID NO: 21), K47Q+R354Q+Y209Q substitution G (G-QQQ) (SEQ ID NO: 26), and K47+R354+Y209 deletion G (G-AKYR) (SEQ ID NO: 27). The results for each construct are shown at pH 7.0 (left bar) and pH 5.0 (right bar) conditions

FIG. 5 is a set of micrographs showing the transduction of SpyTag-VSV-G pseudotyped lentiviruses with or without SpyTag insertion at sites 1, 2, 3, 4, 7, 9, or 11, with (+Linker) and without (-Linker) linkers, in wild type G (G-WT) (SEQ ID NO: 21), K47Q+R354Q+Y209Q substitution G (G-QQQ) (SEQ ID NO: 26), and K47+R354+Y209 deletion G (G-AKYR) (SEQ ID NO: 27) into K562 cells.

FIG. 6 is a western blot showing binding of a VSIV antibody (anti-VSV-G) to SpyTag-VSV-G pseudotyped lentiviruses with or without SpyTag insertion at sites 1, 2, 3, 4, 7, 9, or 11, with (+Linker) and without (-Linker) linkers, in wild type G (G-WT) (SEQ ID NO: 21), K47Q+R354Q+Y209Q substitution G (G-QQQ) (SEQ ID NO: 26), and K47+R354+Y209 deletion G (G-AKYR) (SEQ ID NO: 27). Bands showing both the monomeric (anti-VSV-G) and dimeric (VSV-G dimer) form of VSV-G are labeled.

FIGS. 7A-7B are drawings of the SpyCatcher encoding plasmid (FIG. 7A) and SpyTag encoding plasmid (FIG. 7B) designs.

FIG. 8A is a flowchart showing the steps in the SpyCatcher plasmid transfection into HEK-293T cells and subsequent supernatant collection and concentration for western blotting.

FIGS. 8B and 8C are western blots showing binding of FLAG antibody (α-FLAG) with the cell lysate (FIG. 8B) and supernatant (FIG. 8C) of SpyCatcher transfected cells.

FIG. 9A is a diagram showing the process of creating targeted pseudotyped lentivirus particles by cotransfecting the SpyCatcher plasmid with lentivirus, envelope (SpyTag-G), and packaging plasmids.

FIG. 9B is a diagram showing the process of creating targeted pseudotyped lentivirus particles by transfecting the SpyCatcher plasmid alone and then collecting supernatant and administering it to cells transfected with the lentivirus, envelope (SpyTag-G), and packaging plasmids.

FIG. 10A is a flowchart showing the steps of creating targeted pseudotyped lentivirus particles by cotransfecting the lentivirus and packaging plasmids with different envelope plasmid variants, with or without the SpyCatcher plasmid.

FIGS. 10B and 10C are western blots showing the covalent binding between the recombinant fusion proteins encoded by the envelope plasmid and the SpyCatcher plasmid in the transfected cell lysate (FIG. 10B) and in the collected lentiviruses (FIG. 10C).

FIG. 11A is a set of micrographs showing infection of pseudotyped lentivirus particles with different envelope plasmid variants, with or without the SpyCatcher plasmid, in K562 cells and K562 cells expressing EGFR.

FIGS. 11B and 11C are a set of cytometer images showing cells infected with unconcentrated (FIGS. 11B and 11C) and concentrated (FIGS. 11D and 11E) pseudotyped lentivirus particles with different envelope plasmid variants, with or without the SpyCatcher plasmid, in K562 cells and K562 cells expressing EGFR (FIGS. 11B and 11D) and bar graphs quantifying the K562 cells (left bar) and K562-EGFR cells (right bar) that are GFP positive for each group (FIGS. 11C and 11E).

FIG. 12 is a flowchart showing the process of creating targeted pseudotyped lentivirus particles by transfecting the SpyCatcher plasmid alone and then collecting supernatant and administering it to cells transfected with the lentivirus and packaging plasmids and different envelope plasmid variants.

FIG. 13A is a set of micrograph images showing infection of pseudotyped lentivirus particles with different envelope plasmid variants, with or without the SpyCatcher plasmid, in K562-cKit cells and K562-EGFR cells

FIGS. 13B and 13C are a set of cytometer images showing infection of pseudotyped lentivirus particles with different envelope plasmid variants, with or without the SpyCatcher plasmid, in K562-cKit cells and K562-EGFR cells (FIG. 13B) and a bar graph quantifying the K562-EGFR cells (left bar) and K562-cKit cells (right bar) that are positive for each group (FIG. 13C).

FIGS. 14A and 14B are a set of micrograph images showing infection of pseudotyped lentivirus particles with wild type G (WT) (SEQ ID NO: 21) or SpyTag K47Q+R354Q+Y209Q substitution G (ST-G-QQQ) (SEQ ID NO: 66) bound to SpyCatcher with targeting molecule EGFR scFv (SEQ ID NO: 49), Her2 scFv (SEQ ID NO: 51), or hSCF ligand (SEQ ID NO: 52) in K562, K562-EGFR, K562-Her2, and K562-cKit cells (FIG. 14A) and a bar graph quantifying the K562 cells (left bar), K562-EGFR cells (middle left bar), K562-Her2 cells (middle right bar), and K562-cKit (right bar) that are GFP positive for each group (FIG. 14B).

FIGS. 15A and 15B are a set of cytometer images showing infection of pseudotyped lentivirus particles with wild type G (WT) (SEQ ID NO: 21) or SpyTag K47Q+R354Q+Y209Q substitution G (ST-G-QQQ) (SEQ ID NO: 66) alone or bound to SpyCatcher with targeting molecule UCHT1 (SEQ ID NOs: 45 and 46), TR66-opt (SEQ ID NOs: 43 and 44), or TR66 (SEQ ID NO: 41 ad 42) in Jurkat and Nalm6 cells (FIG. 15A) and a bar graph quantifying the Jurkat cells (left bar) and Nalm6 cells (right bar) that are GFP positive for each group (FIG. 15B).

FIGS. 15C and 15D are a set of cytometer images showing infection of pseudotyped lentivirus particles with wild type G (WT) (SEQ ID NO: 21) or SpyTag K47Q+R354Q+Y209Q substitution G (SEQ ID NO: 66) (ST-G-QQQ) alone or bound to SpyCatcher (SEQ ID NO: 30) with targeting molecule UCHT1 (SEQ ID NOs: 45 and 46), HuM291 (SEQ ID NOs: 37 and 38), OKT3 (SEQ ID NOs: 39 and 40), TR66-opt (SEQ ID NOs: 43 and 44), or TR66 (SEQ ID NO: 41 ad 42) in Jurkat and Nalm6 cells (FIG. 15C) and a bar graph quantifying the Jurkat cells (left bar) and Nalm6 cells (right bar) that are GFP positive for each group (FIG. 15D).

FIGS. 16A-16E show lentivirus particles pseudotyped with wild type G (G-WT) (SEQ ID NO: 21), K47Q+R354Q substitution G (G-QQ) (SEQ ID NO: 25), K47Q+R354Q+Y209Q substitution G (G-QQQ) (SEQ ID NO: 26), or SpyTag K47Q+R354Q+Y209Q substitution G (ST-G-QQQ) (SEQ ID NO: 66) alone or bound to SpyCatcher (SEQ ID NO: 30) with targeting molecule UCHT1 (SEQ ID NOs: 45 and 46) (G-QQQ-ST-UCHT1), TR66-opt (SEQ ID NOs: 43 and 44) (G-QQQ-ST-TR66-opt), or TR66 (SEQ ID NOs: 41 and 42) (G-QQQ-ST-TR66) used to infect human PBMC cultured in hIL2 or hIL-7+hIL-15 containing media. FIGS. 16A and 16B are a set of flow cytometry dot plots showing the percentage of infected PBMCs in media containing hIL-2 (FIG. 16A) or hIL-7 and hIL-15 (FIG. 16B) that express human CD3. FIG. 16C is a flow cytometry graph showing the number of PBMCs in media containing hIL-7 and hIL-15 that are CD25 positive. FIG. 16D is a set of bar graphs showing the mean fluorescence intensity for the PBMCs cultured in media containing hIL-2 (left) or hIL-7 and hIL-15 (right). FIG. 16E is a bar graph showing the percentage of PBMCs cultured in media containing hIL-7 and hIL-15 that were CD25 positive.

FIGS. 17A-17D show western blots of SpyTag containing lentivirus particles with bound SpyCatcher-ligand and unbound SpyCatcher-ligand created using coexpression and mixing approaches. FIG. 17A shows a western blot of lentivirus particles decorated with VSIV-G glycoprotein (G-WT) (SEQ ID NO: 21), SpyTag K47Q+R354Q+Y209Q substitution G glycoprotein alone (ST-G-QQQ) (SEQ ID NO: 66) or with bound SpyCatcher with UCHT1 scFv and FLAG tag (SEQ ID N: 67) (ST-G-QQQ_UCT1-FLAG) created using coexpression and mixing approaches against a FLAG antibody (left) or a VSIV G glycoprotein antibody (middle), and a western blot of lentivirus particles decorated with SpyTag K47Q+R354Q+Y209Q substitution G glycoprotein alone (ST-G-QQQ) (SEQ ID NO: 66) or with bound SpyCatcher with UCHT1 scFv and FLAG tag (SEQ ID NO: 67) (ST-G-QQQ_UCT1-FLAG, and concentrated, unconcentrated, and pelleted SpyCatcher with UCHT1 scFV and FLAG tag alone (right); bands showing SpyTag VSIV G glycoproteins alone (VSV-G), with bound SpyCatcher (* and G-QQQ_Sc-UCHT1), G glycoprotein dimers, and unbound SpyCatcher (Sc-UCHT1) are labeled with arrows. FIG. 17B shows a western blot of lentivirus particles decorated with SpyTag K47Q+R354Q+Y209Q substitution G glycoprotein alone (LV-1_ST-G-QQQ) (SEQ ID NO: 66) or with bound SpyCatcher with UCHT1 scFv and FLAG tag (SEQ ID NO: 67) (LV-2) created using the coexpression approach and concentrated using ultracentrifugation (UC) and PEG8000 (PEG8000) approaches against a FLAG antibody (left) or a VSIV G glycoprotein antibody (right), bands showing SpyTag G glycoproteins alone (VSV-G), with bound SpyCatcher (G-QQQ_Sc-UCHT1), and unbound SpyCatcher (Sc-UCHT1) are labeled with arrows. A loading issue causing a distortion in the p24 bands is indicated with an arrow and label on the right western blot. FIG. 17C is a flow chart showing the process of concentrating lentivirus particles decorated with SpyTag K47Q+R354Q+Y209Q substitution G glycoprotein (SEQ ID NO: 66) bound to SpyCatcher with UCHT1 scFv and FLAG tag (SEQ ID NO: 67). FIG. 17D shows western blot of lentivirus particles decorated with SpyTag K47Q+R354Q+Y209Q substitution G glycoprotein (SEQ ID NO: 66) bound to SpyCatcher with UCHT1 scFv and FLAG tag (SEQ ID NO: 67) before ultracentrifugation (Before UC), after one ultracentrifugation (1^{st} UC), and after a second ultracentrifugation (2^{nd} UC) created using the coexpression approach against a FLAG antibody (left) or a VSIV G glycoprotein antibody (right), bands showing SpyTag VSIV G glycoproteins alone (VSV-G), with bound SpyCatcher (G-QQQ_Sc-UCHT1), and unbound SpyCatcher (Sc-UCHT1) are labeled with arrows.

FIGS. 18A and 18B are western blots against a FLAG antibody and a VSIV G glycoprotein antibody of lentivirus particles decorated with SpyTag K47Q+R354Q+Y209Q substitution VSIV-G glycoprotein (LV ST-VSV-G-QQQ) (SEQ ID NO: 66) bound to SpyCatcher with UCHT1 scFv and FLAG tag (Spycatcher-UCHT1-FLAG) (SEQ ID NO: 67) produced using a mixing approach using a dilution titer of either lentivirus (FIG. 18A) or SpyCatcher (FIG. 18B).

FIGS. 19A and 19B are a flowchart showing the experimental design to test the effect of mixing time on G modification in lentivirus particles (FIG. 19A) and western blots of lentivirus decorated with SpyTag K47Q+R354Q+Y209Q substitution G glycoprotein ((LV) ST-G-QQQ)) (SEQ ID NO: 66) mixed with SpyCatcher with UCHT1 scFV and FLAG tag (Sc-UCHT1-FLAG) (SEQ ID NO: 67) for 0.5, 1, 4, 6, and 24 hours against a VSIV G glycoprotein antibody (left) or a FLAG antibody (right) with short exposure of 2 or 10 seconds (FIG. 19B).

FIGS. 20A-20F show the efficacy of third generation SpyTag and SpyCatcher in lentiviruses pseudotyped with VSIV G glycoproteins and targeting molecules. FIG. 20A shows western blots of the lentivirus particles decorated with VSIV G glycoprotein (G-WT) (SEQ ID NO: 21), third generation SpyTag K47Q+R354Q+Y209Q substitution G glycoprotein alone (ST3-G-QQQ) (SEQ ID NO: 68) or bound to third generation SpyCatcher with UCHT1 scFV and FLAG tag (SEQ ID NO: 69) (ST3-G-QQQ+SC3-UCHT1), third generation SpyTag K47 deletion G glycoprotein alone (ST3-G-ΔK47) (SEQ ID NO: 70) or bound to third generation SpyCatcher with UCHT1 scFV and FLAG tag (SEQ ID NO: 69) (ST3-G-ΔK47+SC3-UCHT1) against a VSIV G glycoprotein antibody or a FLAG antibody. FIG. 20B is a set of micrographs showing Jurkat cells (T cell line) and Nalm6 cells (B cell line) infected with lentivirus particles decorated with VSIV G glycoprotein (WT-G) (SEQ ID NO: 21), first generation SpyTag K47Q+R354Q+Y209Q substitution G glycoprotein (SEQ ID NO: 66) bound to first generation SpyCatcher with UCHT1 scFv and GFP (SEQ ID NO: 67) (SC1-UCHT1+ST1-G-QQQ), or third generation SpyTag K47Q+R354Q+Y209Q substitution G glycoprotein alone (ST3-G-QQQ) (SEQ ID NO: 68) or bound to third generation SpyCatcher with UCHT1 scFv and GFP (SC3-UCHT1+ST3-G-QQQ) (SEQ ID NO: 69). FIGS. 20C and 20D are a set of micrographs showing Jurkat cells (T cell line) and Nalm6 cells (B cell line) infected with lentivirus particles decorated with VSIV G glycoprotein (WT-G) (SEQ ID NO: 21), third generation SpyTag K47Q+R354Q+Y209Q substitution G glycoprotein (SEQ ID NO: 68) bound to third generation SpyCatcher with UCHT1 scFv and GFP (SC3-UCHT1+ST3-G-QQQ) (SEQ ID NO: 69), or third generation SpyTag K47 deletion G glycoprotein (SEQ ID NO: 70) bound to third generation SpyCatcher with UCHT1 scFv and GFP (SC3-UCHT1+ST3-G-ΔK47) (SEQ ID NO: 69) (FIG. 20C), and a bar graph quantifying the Jurkat cells (left bar) and Nalm6 cells (right bar) that are GFP positive for each group (FIG. 20D). FIGS. 20E and 20F are a set of micrographs showing Jurkat cells (T cell line) and Nalm6 cells (B cell line) infected with lentivirus particles decorated with third generation SpyTag K47Q+R354Q+Y209Q substitution G glycoprotein (SEQ ID NO: 68) bound to third generation SpyCatcher with UCHT1 scFv and GFP (SC3-UCHT1+ST3-G-QQQ) (SEQ ID NO: 69) or third generation SpyTag K47 deletion G glycoprotein (SEQ ID NO: 70) bound to third generation SpyCatcher with UCHT1 scFv and GFP (SC3-UCHT1+ST3-G-ΔK47) (SEQ ID NO: 69) (FIG. 20E), and a bar graph quantifying the Jurkat cells (left bar) and Nalm6 cells (right bar) that are GFP positive for each group (FIG. 20F)

FIG. 21 is a set of micrographs showing cells from a human ovarian cancer line (SKOV3) infected with lentivirus particles decorated with VSIV G glycoprotein (WT-G) (SEQ ID NO: 21), first generation SpyTag K47Q+R354Q+Y209Q substitution G glycoprotein (SEQ ID NO: 66) bound to first generation SpyCatcher with UCHT1 scFv and FLAG (SC1-UCHT1+ST1-G-QQQ) (SEQ ID NO: 67), or third generation SpyTag K47Q+R354Q+Y209Q substitution G glycoprotein (SEQ ID NO: 68) bound to third generation SpyCatcher with UCHT1 scFv and FLAG (SC3-UCHT1+ST3-G-QQQ) (SEQ ID NO: 69).

FIGS. 22A-22D are overlapping figures showing alternate rhabdoviral G glycoproteins. FIG. 22A depicts select glycoproteins of the Vesiculovirus, Sprivivirus, Perhabdovirus, Ledantevirus, and Sigmavirus generas. FIG. 22B depicts select glycoproteins of the Ephemerovirus, Tibrovirus, Hapavirus, Curiovirus, Caligrhavirus, Tupavirus, Sripuvirus, and Alphanemrhavirus generas. FIG. 22C depicts select glycoproteins of the Lyssavirus, Almendravirus, and Varicosavirus generas. FIG. 22D depicts select glycoproteins of the Cytorhabdovirus, Dichorhavirus, Nucleorhabdovirus, and Novirhabdovirus generas. Arrows indicate nine glycoproteins from nine different rhabdovirus genera (arrows pointing to Ledantevirus: KM205001 Fukuoka virus (FIG. 22A); Sigmavirus: GQ375258 Drosophila melanogaster sigmavirus (FIG. 22A); Ephemerovirus: AF234533 bovine ephemeral fever virus (FIG. 22B); Tibrovirus: JX297815 Bas-Congo virus (FIG. 22B); Hapavirus: KM205002 Flanders virus (FIG. 22B); Curiovirus: KM204994 Curionopolis virus (FIG. 22B); Tupavirus: AY840978 tupaia rhabdovirus (FIG. 22B); Sripuvirus: KC585008 Niakha virus (FIG. 22B); Almendravirus: KF543749 Puerto Almendras virus (FIG. 22C)).

FIGS. 23A and 23B are a flowchart of the experimental design testing alternative Vesiculovirus G glycoproteins to VSIV G glycoprotein for use in lentivirus (FIG. 23A), and a bar graph showing the number of transduced cells 72 hours post infection with lentivirus decorated with VSIV G glycoprotein (VSV G WT) (SEQ ID NO: 21), SpyTag K1R+K47Q+R354Q+Y209Q substitution Vesiculovirus carajas G glycoprotein (SEQ ID NO: 74) bound to SpyCatcher (SEQ ID NO: 30) (SC:ST CJSV (RQQQ)) with an epidermal growth factor receptor (EGFR) antibody (aEGFR) (SEQ ID NO: 49), a mutant EGF ligand (EGFm123) (SEQ ID NO: 59), or UCHT1, an anti-CD3 scFv (aCD3) (SEQ ID NOs: 45 and 46), SpyTag K1R+K47Q+R354Q+Y209Q substitution Vesiculovirus alagoas G glycoprotein (SEQ ID NO: 75) bound to SpyCatcher (SEQ ID NO: 30) (SC:ST VSAV (RQQQ)) with an EGFR antibody (aEGFR) (SEQ ID NO: 49), a mutant EGF ligand (EGFm123) (SEQ ID NO: 59), or UCHT1, an anti-CD3 scFv (aCD3) (SEQ ID NOs: 45 and 46), or SpyTag K47Q+R354Q+Y209Q substitution Vesiculovirus newjersey G glycoprotein bound (SEQ ID NO: 76) to SpyCatcher (SEQ ID NO: 30) (SC:ST VSVNJ (QQQ)) with an EGFR antibody (aEGFR) (SEQ ID NO: 49), a mutant EGF ligand (EGFm123) (SEQ ID NO: 59), or UCHT1, an anti-CD3 scFv (aCD3) (SEQ ID NOs: 45 and 46), in Jurkat cells (left bar), Jurkat cells expressing EGF receptor (Jurkat EGFR) (middle bar), and Nalm6 cells (right bar) (FIG. 23B).

FIG. 24 is a set of fluorescent micrographs of Jurkat cells transduced with lentivirus containing a GFP-expression cassette and pseudotyped with either VSIV G glycoprotein (LV-G-WT) (SEQ ID NO: 21) or a recombinant fusion protein of first generation SpyTag fused to K47Q+R354Q+Y209Q substitution VSIV G glycoprotein (SEQ ID NO: 66) bound to first generation SpyCatcher fused to UCHT1 scFv (SEQ ID NO: 67) (LV-ST-L-G-QQQ+SC-UCHT1) and cultured in media containing Opti-MEM™ 60% complement active human serum, or 60% heat inactivated (HI) human serum.

### DETAILED DESCRIPTION

In aspects, the present disclosure provides a recombinant fusion protein comprising, consisting essentially of, or consisting of (a) a rhabdoviral G glycoprotein or a functional fragment or derivative thereof and (b) a polypeptide (polypeptide tag) that is capable of forming a covalent bond with an amino acid residue in a second recombinant fusion protein, wherein the polypeptide tag is N-terminal to the rhabdoviral G glycoprotein, and wherein the second recombinant fusion protein comprises a polypeptide (polypeptide catcher) that is capable of forming a covalent bond with an amino acid residue in the polypeptide tag.

In aspects, the present disclosure provides a recombinant protein system comprising, consisting essentially of, or consisting of (a) a recombinant fusion protein comprising, consisting essentially of, or consisting of (1) a rhabdoviral G glycoprotein or a functional fragment or derivative thereof and (2) a polypeptide (polypeptide tag) that is capable of forming a covalent bond with an amino acid residue in a second recombinant fusion protein, wherein the polypeptide tag is N-terminal to the rhabdoviral G glycoprotein, and (b) the second recombinant fusion protein, wherein the second recombinant fusion protein comprises a polypeptide (polypeptide catcher) that is capable of forming a covalent bond with an amino acid residue in the polypeptide tag.

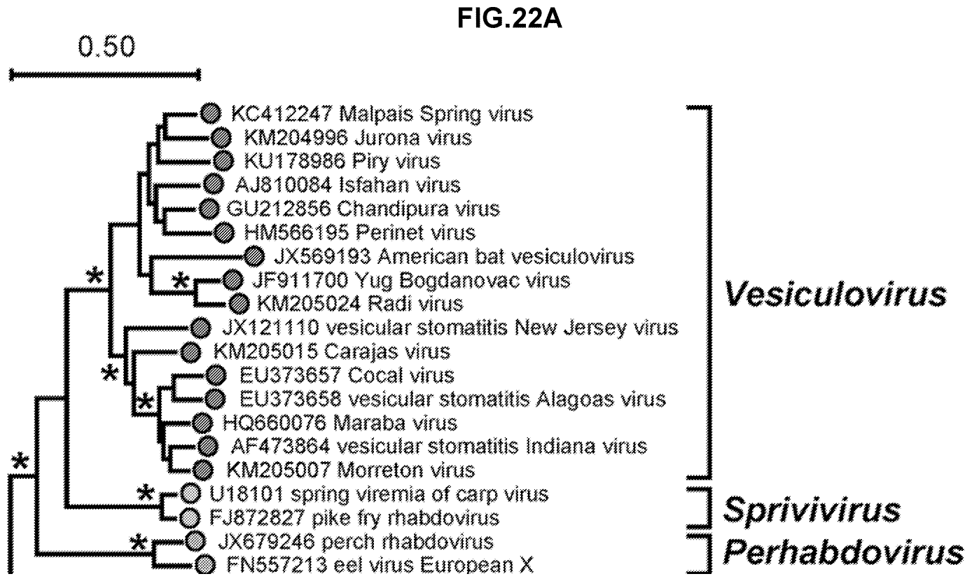

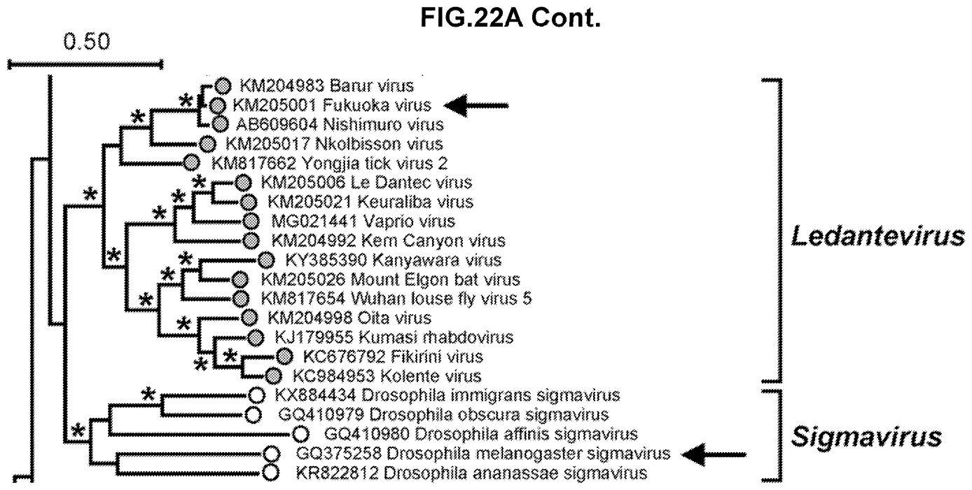

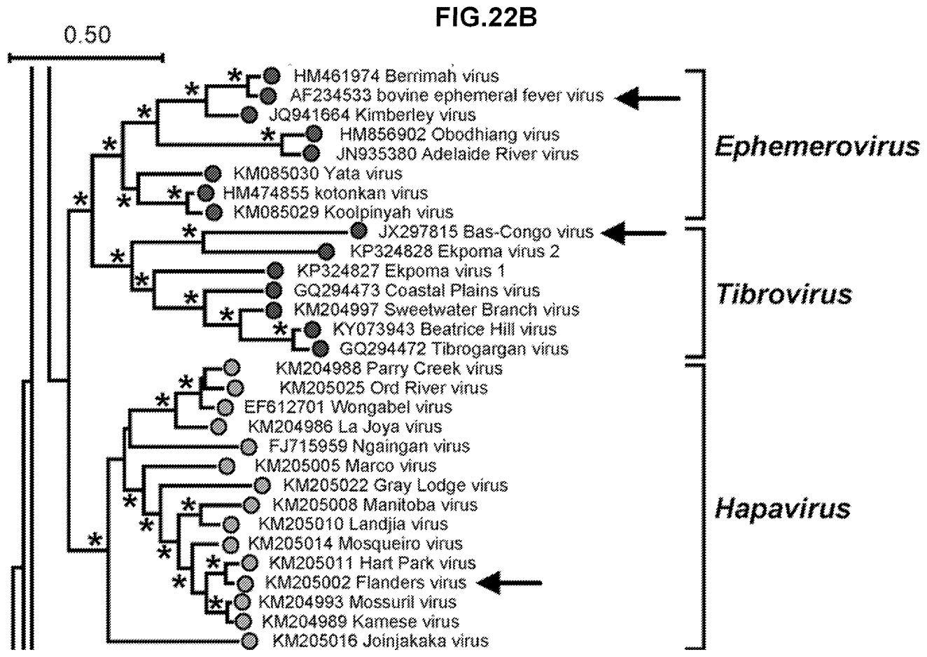

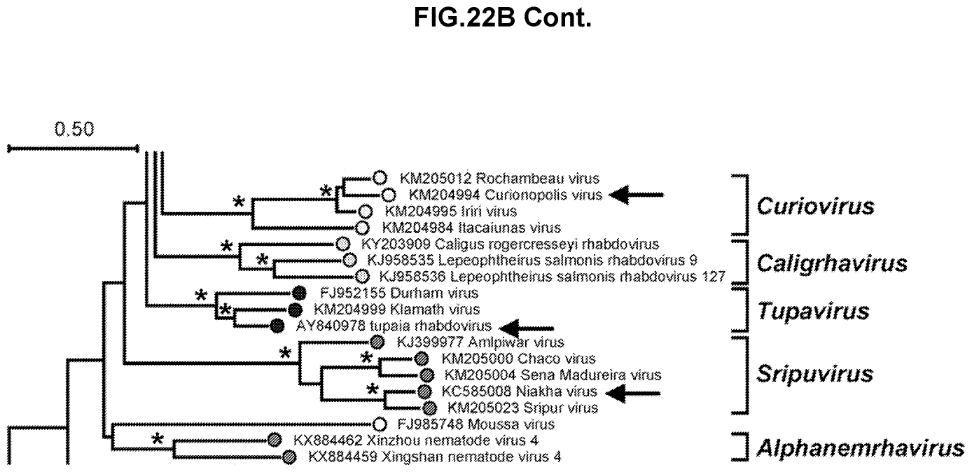

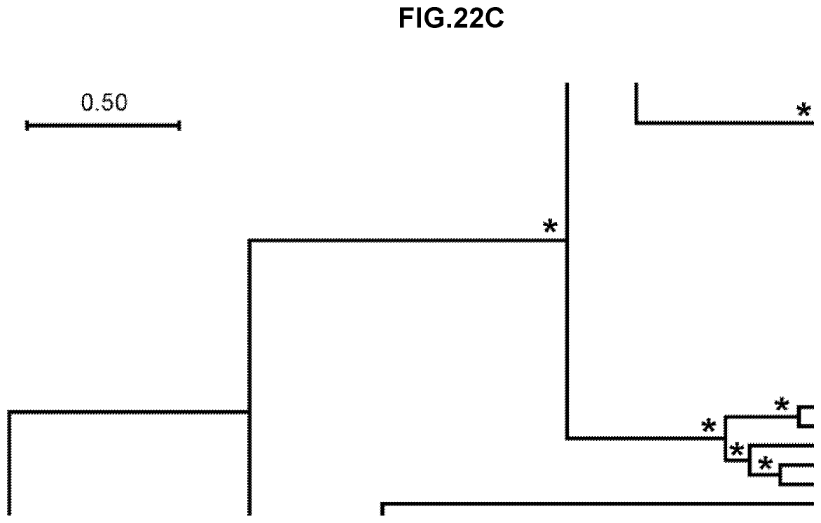

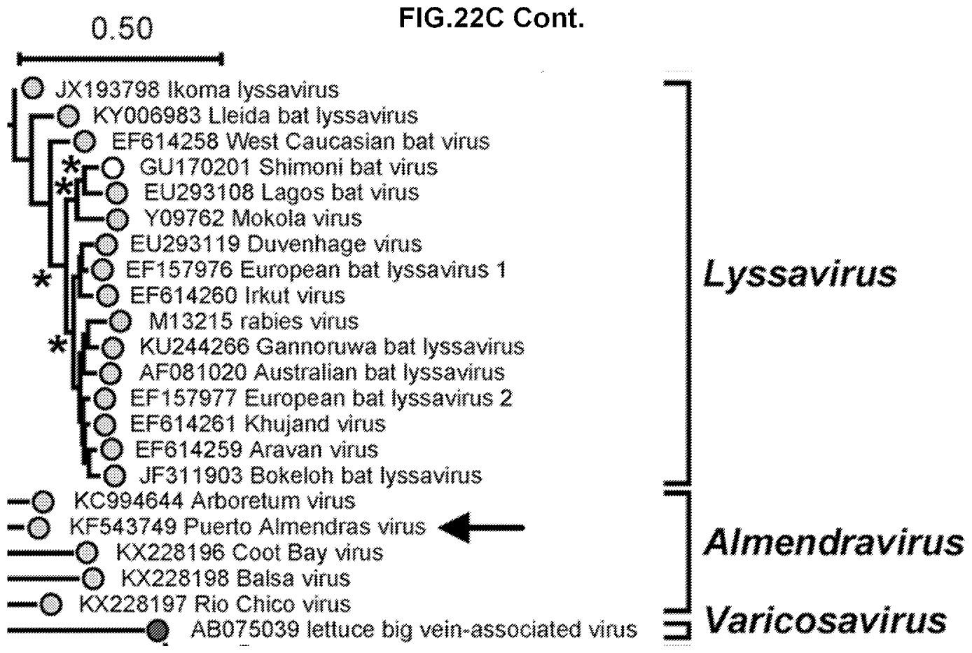

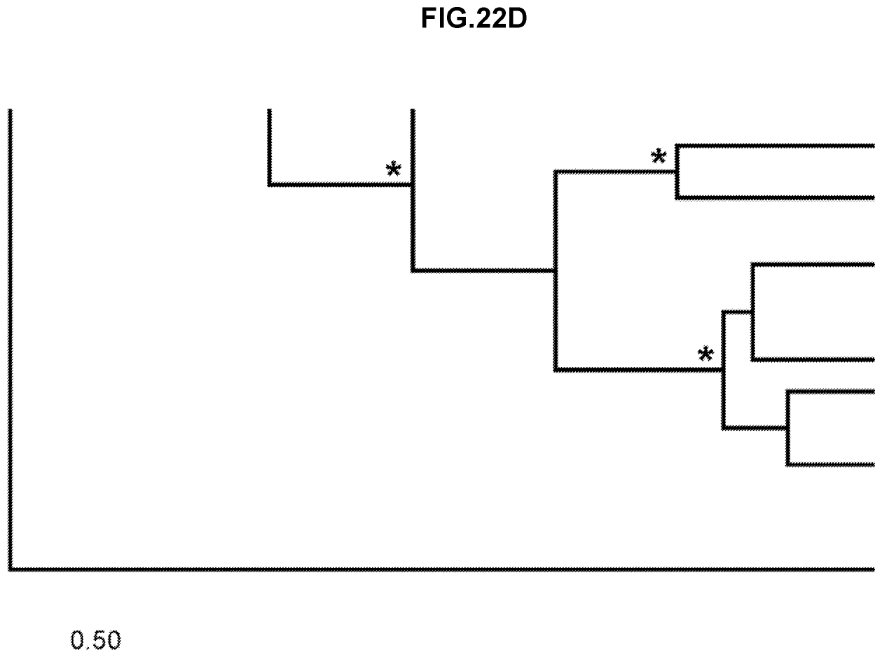

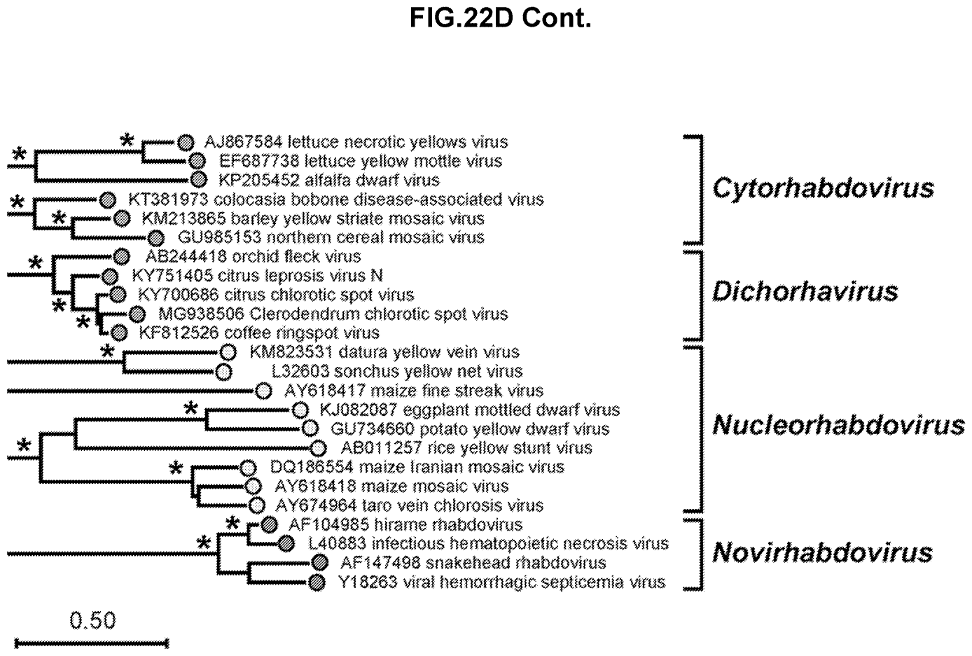

Rhabdoviruses are viruses within the family Rhabdoviridae. The viruses encode proteins denoted N (nucleoprotein), P (phosphoprotein), M (matrix protein), G (glycoprotein), and L (large protein, which is a polymerase), and the viruses appear to have a shape of a bullet when observed using electron microscopy. Dimensions of rhabdovirus virions can range from 100 nm to 430 nm in length and can range from 45 nm to 100 nm in diameter. Exemplary rhabdoviruses, without limitation, that are contemplated herein are listed in FIGS. 22A-22D.

Rhabdoviral G glycoprotein mediates binding of rhabdovirus to a receptor on a cell, which binding then mediates entry of the rhabdovirus into the cell and infection of the cell. A rhabdoviral G glycoprotein or functional fragment or derivative thereof as described herein, including recombinant fusion proteins comprising the rhabdoviral G glycoprotein or functional fragment or derivative thereof, can be used to pseudotype a type of virus that is not the native/natural virus of the rhabdoviral G glycoprotein.

As used herein, a “pseudotype” of a virus, membraned vesicle, enveloped delivery vehicle, enveloped viral particle, or recombinant viral vector, etc. means a virus, membraned vesicle, enveloped delivery vehicle, enveloped viral particle, or recombinant viral vector, etc. comprising a molecule, e.g., a rhabdoviral G glycoprotein or functional fragment or derivative thereof (including a recombinant fusion protein comprising the rhabdoviral G glycoprotein or functional fragment or derivative thereof), that is not typically found in the virus, membraned vesicle, enveloped delivery vehicle, enveloped viral particle, or recombinant viral vector, etc. Such a molecule can have a mutation (e.g., a substitution or deletion) that impacts the tropism of the virus, membraned vesicle, enveloped delivery vehicle, enveloped viral particle, or recombinant viral vector, etc. The impact on tropism can be, e.g., to contribute to, direct, redirect, or completely change, or any combination thereof, the tropism of the virus, membraned vesicle, enveloped delivery vehicle, enveloped viral particle, or recombinant viral vector, etc. when compared to the virus, membraned vesicle, enveloped delivery vehicle, enveloped viral particle, or recombinant viral vector, etc. without the molecule and/or compared to the wild-type virus, membraned vesicle, enveloped delivery vehicle, enveloped viral particle, or recombinant viral vector, etc. The impact on tropism can be, e.g., to target a cell that is different from the cell normally targeted by the virus, membraned vesicle, enveloped delivery vehicle, enveloped viral particle, or recombinant viral vector, etc. and/or to not target a cell that is normally targeted by the virus, membraned vesicle, enveloped delivery vehicle, enveloped viral particle, or recombinant viral vector, etc.

As used herein, “functional fragment” of a rhabdoviral G glycoprotein means a rhabdoviral G glycoprotein that is not a full length rhabdoviral G glycoprotein but is a portion of a parent rhabdoviral G glycoprotein (e.g., a truncated form of a full length rhabdoviral G glycoprotein), where the portion retains the fusion function of the parent full length rhabdoviral G glycoprotein. As used herein, “functional derivative” or “functional variant” of a rhabdoviral G glycoprotein means a rhabdoviral G glycoprotein that has been modified, e.g., by conservative amino acid substitution, where the rhabdoviral G glycoprotein retains the fusion function of the parent full length rhabdoviral G glycoprotein. As used herein, “functional fragment or derivative” and “functional fragment or variant” encompass the meanings of both “functional fragment” and “functional derivative”/“functional variant.” The “fusion function” of a rhabdoviral G glycoprotein means that a rhabdoviral G glycoprotein, when part of a virus, can initiate fusion of the virus with a target cell, e.g., such that the virus can infect the target cell. Such fusion can be due to the interaction of the rhabdoviral G glycoprotein with its natural receptor or the interaction of the rhabdoviral G glycoprotein with a different receptor (e.g., the rhabdoviral G glycoprotein is blinded to its natural receptor, such that the rhabdoviral G glycoprotein is engineered to reduce or abolish its natural receptor binding specificity, and the rhabdoviral G glycoprotein is re-targeted to a new receptor). A functional fragment and/or functional derivative/functional variant of a rhabdoviral G glycoprotein can be within a recombinant fusion protein comprising a rhabdoviral G glycoprotein.

In aspects, the recombinant fusion protein comprises a signal peptide. In aspects, the signal peptide comprises the amino acid sequence of a signal peptide as described herein. As used herein, a “signal peptide” refers to a peptide involved in targeting a glycoprotein to the secretory pathway. In aspects, different signal peptides can be selected to improve glycoprotein targeting to the secretory pathway. In aspects, the signal peptide may be the naturally occurring signal peptide for the rhabdoviral G glycoprotein. In aspects, the signal peptide comprises the amino acid sequence of a signal peptide as described herein. In aspects, the recombinant fusion protein comprises a signal peptide N-terminal to the targeting molecule. In aspects, the signal peptide comprises the amino acid sequence of SEQ ID NO: 60, 121, 122, 123, 253, 256, 257, or 265. In aspects, the signal peptide comprises the amino acid sequence of SEQ ID NO: 60, SEQ ID NO: 121, SEQ ID NO: 122, or SEQ ID NO: 123. In aspects, the recombinant fusion protein comprises a mature form of a rhabdoviral G glycoprotein or functional fragment or derivative thereof, where the signal peptide is not present in the recombinant fusion protein. In aspects, the recombinant fusion protein comprising a rhabdoviral G glycoprotein or functional fragment or derivative thereof does not comprise 1, 2, 3, 4, 5, 6, 7, 8, 9, 10, or more amino acids of the N-terminus of the mature form of the wild-type rhabdoviral G glycoprotein. In aspects, the recombinant fusion protein comprising a rhabdoviral G glycoprotein or functional fragment or derivative thereof does not comprise a signal peptide and also does not comprise 1, 2, 3, 4, 5, 6, 7, 8, 9, 10, or more amino acids of the N-terminus of the mature form of the wild-type rhabdoviral G glycoprotein.

The polypeptide tag is capable of forming a covalent bond with an amino acid residue of another polypeptide. In aspects, the covalent bond capable of being formed is a peptide bond. In aspects, the covalent bond capable of being formed is an isopeptide bond. In aspects, the polypeptide tag is SpyTag (SEQ ID NO: 1), SpyTag002 (SEQ ID NO: 2), SpyTag003 (SEQ ID NO: 3), Isopeptag (SEQ ID NO: 4), or SnoopTag (SEQ ID NO: 5). In aspects, the polypeptide tag is SpyTag (SEQ ID NO: 1).

Previous studies performed using G glycoprotein of the rhabdovirus vesicular stomatitis virus (VSV) have shown that insertion of amino acids are tolerated within certain sites of the amino acid sequence of the VSV-G glycoprotein (Ammayappan et al., J. Virol., 87(24): 13543-13555 (2013), which is incorporated by reference in its entirety): for a 9-residue amino acid sequence, the tolerated insertions were found at seven sites within VSV-G, and even a large 49 amino acid sequence was tolerated at two of the seven sites. Based on those studies, it would have been expected that an amino acid sequence similar in size to the 9-residue amino acid sequence would be tolerated within the same seven sites. However, it unexpectedly has been discovered based on the studies described herein that insertion of a 13-residue amino acid sequence (a polypeptide tag, SpyTag, as described below) was not tolerated (which could be due to, e.g., misfolding, lack of expression in VSV virions, lack of interaction with polypeptide catcher-ligand and could not mediate targeting, etc.) at several of the sites, and only insertion at the site at the N-terminus of VSV-G provided G glycoprotein that retained infectivity.

Additionally, the previous studies showed that insertion of scFv at the N-terminus of the VSV-G resulted in nucleotide changes of the nucleotide encoding the underlying VSV-G, possibly to allow better accommodation of larger amino acid sequences. As provided herein, rhabdoviral G glycoproteins, having a targeting molecule, including an scFv, covalently bonded through a polypeptide tag at the N-terminus of the rhabdoviral G glycoprotein, can retain their amino acid sequences without changes to the nucleotide encoding the rhabdoviral G glycoproteins.

In aspects, the recombinant fusion protein comprises a linker between the polypeptide tag and the rhabdoviral G glycoprotein or functional fragment or derivative thereof. Any suitable linker is contemplated, e.g., as disclosed in Chen et al., Adv. Drug. Deliv. Rev., 65(10): 1357-1369 (2013), which is incorporated herein by reference in its entirety. In aspects, the linker is flexible. Exemplary flexible linkers include but are not limited to e.g.

(SEQ ID NO: 130)
AAASGGSGGGGSGGGGSGP,
(SEQ ID NO: 131)
AAASGGSGGGGSGGGGS,
(SEQ ID NO: 132)
GGGGSGGGGSGGGGSGGGGS,
(SEQ ID NO: 36)
GGGGSGGGGSGGGGS,
(SEQ ID NO: 133)
GGGGSGGGGS,
(SEQ ID NO: 134)
GGGGS,
(SEQ ID NO: 135)
GGGGGGGG,
(SEQ ID NO: 136)
GGGGGG,
(SEQ ID NO: 137)
GSAGSAAGSGEF,
and
(SEQ ID NO: 138)
VPGVGVPGVG.

In aspects, the linker is rigid. Exemplary rigid linkers include, but are not limited to, e.g.,

(SEQ ID NO: 139)
PAPAP,
(SEQ ID NO: 140)
EAAAKEAAAKEAAAK,
(SEQ ID NO: 141)
EAAAKEAAAK,
(SEQ ID NO: 142)
EAAAK,
(SEQ ID NO: 143)
AEAAAKEAAAKEAAAKEAAAKALEAEAAAKEAAAKEAAAKEAAAKA,
(SEQ ID NO: 144)
AEAAAKEAAAKA,
(SEQ ID NO: 145)
ESKYGPPCPPCP,
(SEQ ID NO: 146)
CPPCPAPELLGGPSVF,

and alanine-proline (AP) repeated for a total of 10 to 34 amino acids (SEQ ID NO: 147).

In aspects, the recombinant fusion protein comprises a rhabdoviral G glycoprotein or functional fragment or derivative thereof that is of a Flanders virus glycoprotein (FLAV-G) (SEQ ID NO: 13), a Chandipura virus glycoprotein (CHPV-G) (SEQ ID NO: 14), a Perinet virus glycoprotein (PERV-G) (SEQ ID NO: 15), a Piry virus glycoprotein (PIRYV-G) (SEQ ID NO: 16), a Fukuoka virus glycoprotein (FUKV-G) (SEQ ID NO: 17), a Joinjakaka virus glycoprotein (JOIV-G) (SEQ ID NO: 18), a Kumasi virus glycoprotein (KRV-G) (SEQ ID NO: 19), a Keuraliba virus glycoprotein (KEUV-G) (SEQ ID NO: 20), an Isfahan glycoprotein (ISFV-G), a Jurona glycoprotein (JURV-G), a Mediterranean Bat glycoprotein (MBV-G), a Malpais Spring glycoprotein (MSPV-G), a Radi glycoprotein (RADV-G), a Rhinolophus affinis-G, a Yug Bugdanavoc glycoprotein (YBV-G), a Yinshui Bat glycoprotein (YSBV-G), a Kimberley glycoprotein (KIMV-G), a Kanyawara glycoprotein (KYAV-G), a La Joya glycoprotein (LJV-G), a Mosquiero glycoprotein (MQOV-G), a Parry Creek glycoprotein (PCV-G), a Bas Congo glycoprotein (BASV-G), a Bovine Ephemeral fever glycoprotein (BEFV-G), a Curionopolis glycoprotein (CURV-G), a Drosophila melanogaster sigmavirus glycoprotein (DMelSV-G), a Niakha glycoprotein (NIAV-G), a Puerto almandras glycoprotein (PTAMV-G), or a Tupaia rhabdovirus (TUPTV-G). In aspects, the rhabdoviral G glycoprotein or functional fragment or derivative thereof is of Vesiculovirus glycoprotein or a functional fragment or derivative thereof. In aspects, the rhabdoviral G glycoprotein or functional fragment or derivative thereof is of Vesiculovirus indiana (e.g., SEQ ID NO: 21 or 210), Vesiculovirus newjersey (e.g., SEQ ID NO: 129 or 213), Vesiculovirus carajas (e.g., SEQ ID NO: 23 or 214), Vesiculovirus alagoas (e.g., SEQ ID NO: 24 or 215), Vesiculovirus cocal (e.g., SEQ ID NO: 128 or 216), Vesiculovirus marraba (e.g., SEQ ID NO: 211 or 212), Vesiculovirus morreton (e.g., SEQ ID NO: 217 or 218), or any other rhabdoviral G glycoprotein or functional fragment or derivative thereof as provided herein. In aspects, the rhabdoviral G glycoprotein or functional fragment or derivative thereof is of Vesiculovirus indiana (e.g., SEQ ID NO: 21, 209, 210, or 258), Vesiculovirus newjersey (e.g., SEQ ID NO: 129 or 213), Vesiculovirus carajas (e.g., SEQ ID NO: 23 or 214), Vesiculovirus alagoas (e.g., SEQ ID NO: 24 or 215), Vesiculovirus cocal (e.g., SEQ ID NO: 128 or 216), Vesiculovirus marraba (e.g., SEQ ID NO: 211 or 212), Vesiculovirus morreton (e.g., SEQ ID NO: 217 or 218), or any other rhabdoviral G glycoprotein or functional fragment or derivative thereof as provided herein. In aspects, the rhabdoviral G glycoprotein or functional fragment or derivative thereof is of Vesiculovirus indiana (e.g., SEQ ID NO: 21 or 210). In aspects, the rhabdoviral G glycoprotein or functional fragment or derivative thereof is of Vesiculovirus newjersey (e.g., SEQ ID NO: 129 or 213). In aspects, the rhabdoviral G glycoprotein or functional fragment or derivative thereof is of Vesiculovirus newjersey (SEQ ID NO: 22 or 26). In aspects, the rhabdoviral G glycoprotein or functional fragment or derivative thereof is of Vesiculovirus alagoas (e.g., SEQ ID NO: 124, 125, 285, or 288). In aspects, the rhabdoviral G glycoprotein or functional fragment or derivative thereof is of Vesiculovirus carajas (e.g., SEQ ID NO: 126, 127, 278, or 281) Shown in FIGS. 22A-22D are additional exemplary rhabdoviral G glycoproteins, without limitation, that are contemplated herein.

In aspects, the recombinant fusion protein comprises a rhabdoviral G glycoprotein or functional fragment or derivative thereof that has 99%, 98%, 97%, 96%, 95%, 94%, 93%, 92%, 91%, 90%, 85%, 80% 75%, 70%, 65%, or 60% sequence identity compared to a rhabdoviral G glycoprotein or functional fragment or derivative thereof described herein. In aspects, the rhabdoviral G glycoprotein or functional fragment or derivative thereof contains one or more conservative amino acid substitutions that do not interfere with the fusion function of the rhabdoviral G glycoprotein or functional fragment or derivative thereof.

In aspects, the recombinant fusion protein comprises a rhabdoviral G glycoprotein that is substantially intact. As used herein, a “substantially intact” rhabdoviral G glycoprotein means a rhabdoviral G glycoprotein that is a functional fragment of the rhabdoviral G glycoprotein, where the rhabdoviral G glycoprotein has each of the domains of a rhabdoviral G glycoprotein, the domains as defined in Roche et al., Cell. Mol. Life Sci., 65: 1716-1728 (2008), which is incorporated herein by reference in its entirety.

In aspects, the recombinant fusion protein comprises a rhabdoviral G glycoprotein that is a functional fragment or derivative thereof. In aspects, the cytoplasmic tail of the glycoprotein is truncated, deleted, or replaced with another sequence. Previous work has shown that such truncation or deletion can enhance fusion activity in rhabdoviral G glycoprotein. In aspects, the truncations from the C terminus can be, for example, 1 amino acid, 2 amino acids, 3 amino acids, 4 amino acids, 5 amino acids, 6 amino acids, 7 amino acids, 8 amino acids, 9 amino acids, 10 amino acids, 20 amino acids, 30 amino acids, 40 amino acids, 50 amino acids, 60 amino acids, up to 10 amino acids, up to 20 amino acids, up to 30 amino acids, up to 40 amino acids, up to 50 amino acids, up to 60 amino acids, or more than 60 amino acids. In aspects, the cytoplasmic tail is replaced with another sequence.

In aspects, the recombinant fusion protein comprises a rhabdoviral G glycoprotein or functional fragment or derivative thereof that is engineered to reduce or abolish its natural receptor binding specificity. Reduction in binding specificity can be of any amount, e.g., reduced by 90%, 80%, 70%, 60%, 50%, 40%, 30%, 20%, 10%, 5%, 2%, 1%, or any range between these percentages.

Vesicular stomatitis virus (VSV) is a rhabdovirus, having a natural receptor of the low-density lipoprotein receptor (LDL-R) or very low density lipoprotein receptor (VLDL-R), which are expressed on the cell membrane of many types of cells. In aspects, the rhabdoviral G glycoprotein or functional fragment or derivative thereof is engineered to have a mutation to reduce or abolish its natural receptor binding specificity to LDL-R or VLDL-R, or other receptors which exhibit cross-reactivity to these receptors. In aspects, the rhabdoviral G glycoprotein or functional fragment or derivative thereof comprises a mutation at one or more positions corresponding to H8, K47, Y209, and K354 on the Vesiculovirus indiana glycoprotein (SEQ ID NO: 21). In aspects, the mutation is a substitution of a wild-type amino acid to another amino acid. In aspects, the substitution is with a Q. In aspects, the mutation is a substitution at three or more positions corresponding to H8, K47, Y209, and K354 on the Vesiculovirus indiana glycoprotein (SEQ ID NO: 21). In aspects, the mutation comprises, consists of, or consists essentially of the amino acid sequence of SEQ ID NO: 26, 71, 72, 73, 267, 269, 275, 279, 282, or 286. The US Patent Publication No. 2020/0216502 is incorporated herein by reference in its entirety. In aspects, the mutation is a deletion of a wild-type amino acid. In aspects, the mutation is deletion of one or more positions corresponding to H8, K47, Y209, and K354 on the Vesiculovirus indiana glycoprotein (SEQ ID NO: 21), wherein each deleted amino acid is not present within the amino acid sequence of the glycoprotein. In aspects, the mutation is a single deletion at K47, wherein the K47 amino acid is not present within the amino acid sequence of the glycoprotein. In aspects, the mutation comprises, consists of, or consists essentially of the amino acid sequence of SEQ ID NO: 27, 29, 268, 270, 276, 277, 280, 283, 284, or 287.

In aspects, preferred full length rhabdoviral G glycoproteins, ectodomains, signal peptides, and engineered mutations of the ectodomains to reduce or abolish its natural receptor binding affinity are shown in Table 1. VSIV indicates Vesiculovirus indiana G glycoprotein, VSNJV indicates Vesiculovirus newjersey G glycoprotein, VSCV indicates Vesiculovirus carajas G glycoprotein, VSAV indicates Vesiculovirus alagoas G glycoprotein, VSCOV indicates Vesiculovirus cocal G glycoprotein, WT indicates wild type, 41J indicates residue 41 can be an I or L, KIR indicates a R substitution at residue 1 corresponding to SEQ ID NO: 21, G115A indicates an A substitution at residue 115 corresponding to SEQ ID NO: 21, Delta K47 indicates a deletion at residue 47 corresponding to SEQ ID NO: 21, and K47Q+Y209Q+R354Q indicates Q substitutions at residues 47, 209, and 354 corresponding to SEQ ID NO: 21.

TABLE 1

| | | VSIV | VSNJV | VSNJV | VSCV | VSCV | VSCV | VSAV | VSAV | VSAV | |
|---|---|---|---|---|---|---|---|---|---|---|---|
| | VSIV | (41J) | (WT) | (Variant) | (WT) | (K1R) | G115A | (WT) | (K1R) | G115A | VSCOV |
| Full Length | 210 | 258 | 213 | 266 | 214 | 278 | 281 | 215 | 285 | 288 | 216 |
| G | | | | | | | | | | | |
| Ectodomain | 209 | 21 | 129 | 22 | 23 | 126 | 127 | 24 | 124 | 125 | 128 |
| G | | | | | | | | | | | |
| Signal | 60 | 60 | 265 | 121 | 123 | 123 | 123 | 122 | 122 | 122 | 253 |
| Peptide | | | | | | | | | | | |
| Delta K47 | 290 | 27 | 268 | 29 | 276 | 277 | 280 | 283 | 284 | 287 | 270 |
| K47Q + | 289 | 26 | 267 | 73 | 275 | 71 | 279 | 282 | 72 | 286 | 269 |
| Y209Q + | | | | | | | | | | | |
| R354Q | | | | | | | | | | | |

It has been found that a Vesiculovirus indiana rhabdoviral G glycoprotein having a deletion of K47 reduces or abolishes the natural receptor binding specificity of the rhabdoviral G glycoprotein when the rhabdoviral G glycoprotein while unexpected retaining full function of the G protein in other respects. In particular, this feature is seen when incorporated into a lentiviral vector. However, when used in a VSV vector, there is second site mutation (F405I) generated in the VSV-G protein during the virus amplification. This mutation leads to the loss of detargeting effect caused by the deletion on K47 residue. Without wishing to be bound by theory, this phenomenon may be due to VSV being a replicating virus, whereas lentivirus is a non-replicating virus.

Vesiculovirus indiana glycoprotein having H8 and/or K47 deletions, when incorporated into a lentiviral system, can support production of functional pseudotyped lentivirus and successful cell transduction. The H8 and/or K47 deletions, but not Y209 or R354 deletions or Y209/R354 deletion combinations, demonstrated generation and rescue of pseudotyped lentivirus with low to no LDL-R background binding.

In aspects, the recombinant fusion protein comprises a rhabdoviral G glycoprotein or functional fragment or derivative thereof that comprises one or more viral titer increasing mutations. In aspects, the one or more viral titer increasing mutations is one or both of M184T and F250L in SEQ ID NO: 21 (or the positions corresponding to M184T and F250L). Other exemplary mutations, without limitation, for increasing viral titer include those described in US Patent Publication No. 2022/0162266, which is incorporated herein by reference in its entirety. The mutations include H22N and S422I in the ectodomain of Vesiculovirus indiana G glycoprotein (SEQ ID NO: 21) (or substitutions in positions corresponding to these in other rhabdoviral G glycoproteins).

Amino acid positions of other rhabdoviral G glycoproteins or functional fragments or derivatives thereof that are “positions corresponding to” the amino acids discussed above can be determined using a global sequence alignment algorithm (see, e.g., Madeira et al., Nuc. Acids Res., 50(W1): W276-W279 (2022), which is incorporated herein by reference in its entirety) comparing the base rhabdoviral G glycoprotein to another rhabdoviral G glycoprotein.

In aspects, the rhabdoviral G glycoprotein or functional fragment or derivative thereof is mutated to reduce or abolish protease cleavage.

The polypeptide catcher is capable of forming a covalent bond with an amino acid residue of another polypeptide. In aspects, the covalent bond capable of being formed is a peptide bond. In aspects, the covalent bond capable of being formed is an isopeptide bond. In aspects, the polypeptide tag is covalently bonded to the polypeptide catcher. In aspects, the formation of the covalent bond is spontaneous. In aspects, the covalent bond is a peptide bond. In aspects, the covalent bond is an isopeptide bond. In aspects, the polypeptide tag and the polypeptide catcher are the pair SpyTag and SpyCatcher (SEQ ID NO: 1 and SEQ ID NO: 30), SpyTag002 and SpyCatcher002 (SEQ ID NO: 2 and SEQ ID NO: 31), SpyTag003 and SpyCatcher003 (SEQ ID NO: 3 and SEQ ID NO: 32), SpyTag and KTag (SEQ ID NO: 1 and SEQ ID NO: 33), Isopeptag and pilin-C(SEQ ID NO: 4 and SEQ ID NO: 34), or SnoopTag and SnoopCatcher (SEQ ID NO: 5 and SEQ ID NO: 35). In aspects, the polypeptide tag and the polypeptide catcher are the pair SpyTag003 and SpyCatcher003 (SEQ ID NO: 3 and SEQ ID NO: 32).

In aspects, the second recombinant fusion protein comprises a targeting molecule. As used herein, the term “targeting molecule” refers to a binding moiety (that can bind or that can be bound), such as a natural ligand, antibody, multispecific binding molecule, or others known in the art. In aspects, the targeting molecule is an antibody or portion thereof. In aspects, the portion thereof is an antigen-binding fragment. In aspects, the antibody or portion thereof is a single chain fragment variable (scFv), an affibody, a diabody, a minibody, a nanobody, a single-domain antibody (sdAb), or a single heavy chain antibody. Also contemplated herein are monoclonal antibodies, human antibodies, humanized antibodies, chimeric antibodies, single chain antibodies, Fab fragments, F(ab′) fragments, disulfide-linked Fvs (sdFv), intrabodies, and anti-idiotypic (anti-Id) antibodies (including, e.g., anti-Id antibodies to antigen specific TCR), Ig-DARTS, epitope-binding fragments of any of the above, and what is described in US Patent Publication No. 2007/0004909 and US Patent Publication No. 2009/0060910, each of which is incorporated herein by reference in their entireties). Also contemplated are immunoglobulin molecules and immunologically active fragments of immunoglobulin molecules, i.e., molecules that contain an antigen binding site. Immunoglobulin molecules can be of any type (e.g., IgG, IgE, IgM, IgD, IgA and IgY), class (e.g., IgGI, IgG2, IgG3, IgG4, IgAI and IgA2) or subclass. In aspects, the targeting molecule is an antibody comprising a variable domain that binds a cell surface protein on a target cell and a heavy chain constant domain. In aspects, the targeting molecule is an antibody comprising a variable domain that binds a cell surface protein on a target cell and an IgG heavy chain constant domain. In aspects, the targeting molecule is an antibody comprising a variable domain that binds a cell surface protein on a target cell and an IgG heavy chain constant domain.

In aspects, a targeting molecule specifically binds a cell surface molecule, e.g., an oligosaccharide, a receptor, cell surface marker, etc., expressed on the surface of a mammalian (e.g., human) eukaryotic cell. In aspects, a targeting molecule binds a (e.g., human) liver cell, a (e.g., human) brain cell, a (e.g., human) T cell, a (e.g., human) kidney cell, a (e.g., human) intestinal cell, a (e.g., human) lung cell, a (e.g., human) cancerous cell, or a (e.g., human) cell infected with heterologous pathogen. In aspects, the targeting molecule binds a receptor expressed by a (e.g., human) liver cell, e.g., an asialoglycoprotein receptor, e.g., hASGR1. In aspects, the targeting molecule binds a molecule expressed by a (e.g., human) neuronal cell, e.g., GABA, transferrin, etc. In aspects, the targeting molecule binds a molecule expressed by a (e.g., human) T cell, e.g., CD3, e.g., CD3c. In aspects, the targeting molecule binds CD63. In aspects, the targeting molecule binds a molecule expressed by a (e.g., human) hematopoietic stem cell, e.g., CD34. In aspects, the targeting molecule binds a molecule expressed by a (e.g., human) kidney cell. In aspects, the targeting molecule binds a molecule expressed by a (e.g., human) muscle cell, e.g., an integrin. In aspects, the targeting molecule binds a molecule expressed by a (e.g., human) cancerous cell, e.g., a tumor associated antigen, e.g., adipophilin, AIM-2, ALDHIAI, alpha-actinin-4, alpha-fetoprotein (“AFP”), ARTCI, B-RAF, BAGE-1, BCLX (L), BCR-ABL fusion protein b3a2, beta-catenin, BING-4, CA-125, CALCA, carcinoembryonic antigen (“CEA”), CASP-5, CASP-8, CD274, CD45, Cdc27, CDK12, CDK4, CDKN2A, CEA, CLPP, COA-1, CPSF, CSNKIAI, CTAGI, CTAG2, cyclin DI, Cyclin-A1, dek-can fusion protein, DKKI, EFTUD2, Elongation factor 2, ENAH (hMena), Ep-CAM, EpCAM, EphA3, epithelial tumor antigen (“ETA”), ETV6-AML1 fusion protein, EZH2, E6, E7, FGF5, FLT3-ITD, FNI, G250/MN/CAIX, GAGE-1,2,8, GAGE-3,4,5,6,7, GAS7, glypican-3, GnTV, gp100/Pmell 7, GPNMB, HAUS3, Hepsin, HER-2/neu, HERV-K-MEL, HLA-Al1, HLA-A2, HLA-DOB, hsp70-2, IDOI, IGF2B3, IL13Ralpha2, Intestinal carboxyl esterase, K-ras, Kallikrein 4, KIF20A, KK-LC-1, KKLCI, KM-HN-1, KMHNI also known as CCDCI 10, LAGE-I, LDLR-fucosyltransferaseAS fusion protein, Lengsin, M-CSF, MAGE-A1, MAGE-AIO, MAGE-A12, MAGE-A2, MAGE-A3, MAGE-A4, MAGE-A6, MAGE-A9, MAGE-C1, MAGE-C2, malic enzyme, mammaglobin-A, MART2, MATN, MCIR, MCSP, mdm-2, MEI, Melan-A/MART-1, Meloe, Midkine, MMP-2, MMP-7, MUCI, MUC5AC, mucin, MUM-I, MUM-2, MUM-3, Myosin, Myosin class I, N-raw, NA88-A, neo-PAP, NFYC, NY-BR-I, NY-ESO-1/LAGE-2, OAI, OGT, OS-9, P polypeptide, p53, PAP, PAX5, PBF, pml-RARalpha fusion protein, polymorphic epithelial mucin (“PEM”), PPPIR3B, PRAME, PRDX5, PSA, PSMA, PTPRK, RAB38/NY-MEL-1, RAGE-I, RBAF600, RGS5, RhoC, RNF43, RU2AS, SAGE, secemin 1, SIRT2, SNRPDI, SOXI0, Spl7, SPA17, SSX-2, SSX-4, STEAPI, survivin, SYT-SSXI or -SSX2 fusion protein, TAG-I, TAG-2, Telomerase, TGF-betaRII, TPBG, TRAG-3, Triosephosphate isomerase, TRP-1/gp75, TRP-2, TRP2-INT2, tyrosinase, tyrosinase (“TYR”), VEGF, WTI, XAGE-lb/GAGED2a, Kras, NYESOI, HPV E2, HPV E6, HPV E7, WT-I antigen (in lymphoma and other solid tumors), ErbB receptors, Melan A [MARTI], gp 100, tyrosinase, TRP-1/gp 75, and TRP-2 (in melanoma); MAGE-I and MAGE-3 (in bladder, head and neck, and non-small cell carcinoma); HPV EG and E7 proteins (in cervical cancer); Mucin [MUC-1](in breast, pancreas, colon, and prostate cancers); prostate-specific antigen [PSA](in prostate cancer); carcinoembryonic antigen [CEA](in colon, breast, and gastrointestinal cancers), and such shared tumor-specific antigens as MAGE-2, MAGE-4, MAGE-6, MAGE-IO, MAGE-12, BAGE-1, CAGE-1,2,8, CAGE-3 TO 7, LAGE-I, NY-ESO-1/LAGE-2, NA-88, GnTV, TRP2-INT2, etc. In aspects, the targeting molecule binds E6 and/or E7. In aspects, the targeting molecule binds Her2. In aspects, the targeting molecule binds CD63. In aspects, the targeting molecule binds human glucagon receptor (hGCGR). In aspects, the targeting molecule binds human ectonucleoside triphosphate diphosphohydrolase 3 (hENTPD3).

In aspects, the targeting molecule is a polypeptide antibody construct. In aspects, the polypeptide antibody construct is agonistic to CD3. In aspects, the polypeptide antibody construct comprises a single chain variable fragment (scFv). In aspects, the scFv has the VL N-terminal to VH. In aspects, the scFv has the VH N-terminal to VL. In aspects, the scFv is UCHT1, HuM291, OKT3, or TR66. In aspects, the scFv is a humanized UCHT1. In aspects, the scFv comprises a variable heavy chain (VH) comprising, consisting essentially of, or consisting of the amino acid sequence of SEQ ID NO: 46 and a variable light chain (VL) comprising, consisting essentially of, or consisting of the amino acid sequence of SEQ ID NO: 45. In aspects, the VH and VL are separated by a flexible linker. In aspects, the flexible linker is SEQ ID NO: 36. In aspects, the scFv is TR66 and wherein the TR66 is codon optimized for expression in human and wherein the scFv comprises a variable heavy chain (VH) comprising, consisting essentially of, or consisting of the amino acid sequence of SEQ ID NO: 44 and a variable light chain (VL) comprising, consisting essentially of, or consisting of the amino acid sequence of SEQ ID NO: 43. In aspects, the scFv is TR66opt and wherein the scFv comprises a variable heavy chain (VH) comprising, consisting essentially of, or consisting of the amino acid sequences of a complementarity determining region 1 (CDR1) of SEQ ID NO: 247, a complementarity determining region 2 (CDR2) of SEQ ID NO: 248, and a complementarity determining region 3 (CDR3) of SEQ ID NO: 249, and a variable light chain (VL) comprising, consisting essentially of, or consisting of the amino acid sequences of a CDR1 of SEQ ID NO: 250, a CDR2 of SEQ ID NO: 251, and a CDR3 of SEQ ID NO: 252. In aspects, the scFv is UCHT1 wherein the scFv comprises a variable heavy chain (VH) comprising, consisting essentially of, or consisting of the amino acid sequence of SEQ ID NO: 46 and a variable light chain (VL) comprising, consisting essentially of, or consisting of the amino acid sequence of SEQ ID NO: 45. In aspects, the scFv is UCHT1 and wherein the scFv comprises a variable heavy chain (VH) comprising, consisting essentially of, or consisting of the amino acid sequences of a CDR1 of SEQ ID NO: 223, a CDR2 of SEQ ID NO: 224, and a CDR3 of SEQ ID NO: 225, and a variable light chain (VL) comprising, consisting essentially of, or consisting of the amino acid sequences of a CDR1 of SEQ ID NO: 226, a CDR2 of SEQ ID NO: 227, and a CDR3 of SEQ ID NO: 228. In aspects, the scFv is HuM291 wherein the scFv comprises a variable heavy chain (VH) comprising, consisting essentially of, or consisting of the amino acid sequence of SEQ ID NO: 38 and a variable light chain (VL) comprising, consisting essentially of, or consisting of the amino acid sequence of SEQ ID NO: 37. In aspects, the scFv is Hum291 and wherein the scFv comprises a variable heavy chain (VH) comprising, consisting essentially of, or consisting of the amino acid sequences of a CDR1 of SEQ ID NO: 235, a CDR2 of SEQ ID NO: 236, and a CDR3 of SEQ ID NO: 237, and a variable light chain (VL) comprising, consisting essentially of, or consisting of the amino acid sequences of a CDR1 of SEQ ID NO: 238, a CDR2 of SEQ ID NO: 239, and a CDR3 of SEQ ID NO: 240. In aspects, the scFv is OKT3 wherein the scFv comprises a variable heavy chain (VH) comprising, consisting essentially of, or consisting of the amino acid sequence of SEQ ID NO: 40 and a variable light chain (VL) comprising, consisting essentially of, or consisting of the amino acid sequence of SEQ ID NO: 39. In aspects, the scFv is OKT3 and wherein the scFv comprises a variable heavy chain (VH) comprising, consisting essentially of, or consisting of the amino acid sequences of a CDR1 of SEQ ID NO: 241, a CDR2 of SEQ ID NO: 242, and a CDR3 of SEQ ID NO: 243, and a variable light chain (VL) comprising, consisting essentially of, or consisting of the amino acid sequences of a CDR1 of SEQ ID NO: 244, a CDR2 of SEQ ID NO: 245, and a CDR3 of SEQ ID NO: 246. In aspects, the scFv is TR66 wherein the scFv comprises a variable heavy chain (VH) comprising, consisting essentially of, or consisting of the amino acid sequence of SEQ ID NO: 42 and a variable light chain (VL) comprising, consisting essentially of, or consisting of the amino acid sequence of SEQ ID NO: 41. In aspects, the scFv is TR66 and wherein the scFv comprises a variable heavy chain (VH) comprising, consisting essentially of, or consisting of the amino acid sequences of a CDR1 of SEQ ID NO: 229, a CDR2 of SEQ ID NO: 230, and a CDR3 of SEQ ID NO: 231, and a variable light chain (VL) comprising, consisting essentially of, or consisting of the amino acid sequences of a CDR1 of SEQ ID NO: 232, a CDR2 of SEQ ID NO: 233, and a CDR3 of SEQ ID NO: 234.

In aspects, the targeting molecule is an anti-cKit binding domain or a functional fragment or derivative thereof capable of selectively binding to the c-KIT receptor. In aspects, the anti-cKit binding domain comprises an anti-cKit 2D1 scFv or a functional fragment or derivative thereof wherein the scFv comprises a variable heavy chain (VH) comprising, consisting essentially of, or consisting of the amino acid sequence of SEQ ID NO: 195 and a variable light chain (VL) comprising, consisting essentially of, or consisting of the amino acid sequence of SEQ ID NO: 196.

In aspects, the targeting molecule is a ligand. In aspects, the ligand is epidermal growth factor (EGF), a mutant EGF (EFGm123) (SEQ ID NO: 59), stem cell factor (SCF) (e.g., SEQ ID NOs: 52-54), thrombopoietin (TPO) (SEQ ID NO: 55), human hepatocyte growth factor (HGF) (SEQ ID NO: 56), or type 1 insulin-like growth factor (IGF1) (SEQ ID NO: 58). In aspects, the targeting molecule is a single chain variable fragment (scFv). In aspects, the scFv binds epidermal growth factor receptor (EGFR) (SEQ ID NO: 49), human epidermal growth factor receptor 2 (Her2) (SEQ ID NO: 51), cluster of differentiation 3 (CD3), or cluster of differentiation 117 (cKit), mucin-16 (MUC16), B cell maturation antigen (BCMA), or Nectin4.

The targeting molecule can target any suitable cell type. Non-limiting examples of cell types include hematopoietic stem cells (HSC), T cells, monocytes, and NK cells. For HSC, non-limiting examples of markers that can be targeted by the targeting molecule include, e.g., one or more of CD34, CD133, and CD90 (Thy-1). For T cells, non-limiting examples of markers that can be targeted by the targeting molecule include, e.g., one or more of CD3, CD4, and CD8. For monocytes, non-limiting examples of markers that can be targeted by the targeting molecule include, e.g., one or both of CD14 and CD1 lb. For NK cells, non-limiting examples of markers that can be targeted by the targeting molecule include, e.g., one or both of CD56 and NKG2D.

In aspects, the second recombinant fusion protein comprises a linker between the targeting molecule and the polypeptide catcher. In aspects, the linker is flexible. Exemplary flexible linkers include, but are not limited to, e.g.,

(SEQ ID NO: 130)
AAASGGSGGGGSGGGGSGP,
(SEQ ID NO: 131)
AAASGGSGGGGSGGGGS,
(SEQ ID NO: 132)
GGGGSGGGGSGGGGSGGGGS,
(SEQ ID NO: 36)
GGGGSGGGGSGGGGS,
(SEQ ID NO: 133)
GGGGSGGGGS,
(SEQ ID NO: 134)
GGGGS,
(SEQ ID NO: 135)
GGGGGGGG,
(SEQ ID NO: 136)
GGGGGG,
(SEQ ID NO: 137)
GSAGSAAGSGEF,
and
(SEQ ID NO: 138)
VPGVGVPGVG.

In aspects, the linker is rigid. Exemplary rigid linkers include, but are not limited to, e.g.,

(SEQ ID NO: 139)
PAPAP,
(SEQ ID NO: 140)
EAAAKEAAAKEAAAK,
(SEQ ID NO: 141)
EAAAKEAAAK,
(SEQ ID NO: 142)
EAAAK,
(SEQ ID NO: 143)
AEAAAKEAAAKEAAAKEAAAKALEAEAAAKEAAAKEAAAKEAAAKA,
(SEQ ID NO: 144)
AEAAAKEAAAKA,
(SEQ ID NO: 145)
ESKYGPPCPPCP,
(SEQ ID NO: 146)
CPPCPAPELLGGPSVF,

alanine-proline (AP) repeated for a total of 10 to 34 amino acids (SEQ ID NO: 147).

In aspects, the targeting molecule is N terminal to the polypeptide catcher. In aspects, the targeting molecule is C terminal to the polypeptide catcher.

In aspects, the second recombinant fusion protein comprises a signal peptide. In aspects, the signal peptide comprises the amino acid sequence of a signal peptide as described herein. In aspects, the signal peptide comprises the amino acid sequence of SEQ ID NO: 61. In aspects, the signal sequence is cleaved off.

In aspects, the second recombinant fusion protein comprises a tag for western detection or purification. In aspects, the tag is a tag for western detection. In aspects, the tag is a tag for purification. In aspects the tag is a FLAG tag (SEQ ID NO: 62), GFP, or others.

In aspects, the present disclosure provides a polynucleotide that encodes any of the proteins and polypeptides described herein, including any rhabdoviral G glycoprotein or functional fragment or derivatives thereof, and their recombinant fusion proteins, described herein.

In aspects, the present disclosure provides a membraned vesicle comprising, consisting essentially of, or consisting of a recombinant fusion protein as described herein or a recombinant protein system as described herein.

As used herein, a “membraned vesicle” is a vesicle bound (delimited) by a lipid bilayer. In aspects, the membraned vesicle is either naturally-derived or engineered. In aspects, the vesicle is a gesicle or an exosome. A gesicle can be as described in Mangeot et al., Mol. Ther., 19: 1656-1666 (2011), which is incorporated herein by reference in its entirety, where overexpression of glycoprotein of VSV-G in human cells induced release of fusogenic vesicles. A membraned vesicle can be cell-derived and thus be a cell-derived enveloped particle (CDEP).

In aspects, the present disclosure provides an enveloped viral particle comprising, consisting essentially of, or consisting of a recombinant fusion protein as described herein or a recombinant protein system as described herein.

As used herein, an “enveloped viral particle” is a vesicle bound (delimited) by a lipid bilayer and has the ability to infect a cell and produce additional enveloped viral particles. An enveloped viral particle can include one or more components of a virus, in addition to a rhabdoviral G glycoprotein or functional fragment or derivative thereof or recombinant fusion protein thereof. Exemplary components of a virus include, without limitation, the gag, po, or env gene or gene product of lentivirus. A “virus-like particle” as used herein means an enveloped viral particle that cannot produce additional virus-like particles (e.g., is replication-incompetent).

In aspects, membraned vesicles, cell-derived enveloped particles, enveloped viral particles, and virus-like particles can encapsulate proteins, lipids, nucleic acids, etc. for delivery, such that membraned vesicles, cell-derived enveloped particles, enveloped viral particles, and virus-like particles can be considered “enveloped delivery vehicles.”

In aspects, an enveloped delivery vehicle, a membraned vesicle, or an enveloped viral particle comprises a mixed trimer, wherein the mixed trimer comprises (a) at least one recombinant fusion protein, wherein the polypeptide tag of the recombinant fusion protein is covalently bound to the polypeptide catcher of the second recombinant fusion protein, and (b) at least one (i) recombinant fusion protein, wherein the polypeptide tag of the recombinant fusion protein is not covalently bound to a polypeptide catcher or (ii) rhabdoviral G glycoprotein or functional fragment or derivative thereof that is not within a recombinant fusion protein. Without being bound by theory, decreasing the number of units in a recombinant rhabdoviral G glycoprotein trimer that are decorated with a covalently bound targeting molecule, particularly a large targeting molecule, better allows the transformation of the trimer from a prefusion to a fusion conformation. In aspects, an enveloped delivery vehicle described herein comprises a mixed trimer.

In aspects, the enveloped delivery vehicle, membraned vesicle, or enveloped viral particle comprises an unmixed trimer, wherein the unmixed trimer comprises only rhabdoviral G glycoprotein or a functional fragment or derivative thereof that is not within a recombinant fusion protein. In aspects, an enveloped delivery vehicle described herein comprises an unmixed trimer.

In aspects, an enveloped delivery vehicle, a membraned vesicle, or an enveloped viral particle comprises the maximum number of the recombinant fusion proteins possible to be accommodated within the membrane of the enveloped delivery vehicle, membraned vesicle, or enveloped viral particle, wherein the polypeptide tag of the recombinant fusion protein is covalently bound to the polypeptide catcher of the second recombinant fusion protein. In aspects, an enveloped delivery vehicle, a membraned vesicle, or an enveloped viral particle comprises less than the maximum number of the recombinant fusion proteins possible to be accommodated within the membrane of the enveloped delivery vehicle, membraned vesicle, or enveloped particle, wherein the polypeptide tag of the recombinant fusion protein is covalently bound to the polypeptide catcher of the second recombinant fusion protein. Less than the maximum amount can be of any amount less than 100%, e.g., 90%, 80%, 70%, 60%, 50%, 40%, 30%, 20%, 10%, 5%, 2%, or 1%, or any range between these percentages. In aspects, any enveloped delivery vehicle as described herein comprises the maximum number of the recombinant fusion proteins possible to be accommodated within the membrane.

In aspects, the present disclosure provides a recombinant viral vector comprising, consisting essentially of, or consisting of a nucleotide encapsulated by an enveloped delivery vehicle as described herein, a membraned vesicle as described herein, or an enveloped viral particle as described herein.

In aspects, the present disclosure provides a composition comprising, consisting essentially of, or consisting of a pharmaceutically acceptable carrier and an enveloped delivery vehicle as described herein, a membraned vesicle as described herein, an enveloped viral particle as described herein, or a recombinant viral vector as described herein. Pharmaceutically acceptable carriers are well-known in the art.

In aspects, the present disclosure provides a method of delivering a payload to a target cell, the method comprising, consisting essentially of, or consisting of contacting the target cell with a membraned vesicle as described herein, an enveloped viral particle as described herein, an enveloped delivery vehicle as described herein, a recombinant viral vector as described herein, or a composition as described herein. In aspects, the target cell in in vitro or ex vivo. In aspects, the target cell in in vivo. In aspects, the payload is a nucleotide sequence encoding a chimeric antigen receptor (CAR) comprising, consisting essentially of, or consisting of an antigen binding domain, a transmembrane domain, and an intracellular signaling domain. In aspects, the payload is a nucleotide sequence encoding a transgene.

In aspects, the present disclosure provides a retroviral vector expression system comprising, consisting essentially of, or consisting of one or more nucleotide sequences encoding a recombinant fusion protein as described herein or a recombinant protein system as described herein. In aspects, the retroviral vector expression system comprises a vector construct and helper constructs that are each on separate plasmids. In aspects, the retroviral vector expression system is a lentirival vector expression system, a foamy vector expression system, or a respiratory syncytial virus (RSV). A rhabdoviral G glycoprotein or functional fragment or derivative thereof as described herein can be expressed within a retroviral vector expression system to pseudotype the vector and alter the tropism of the vector.

As used herein, a “vector construct” comprises a nucleic acid of interest, e.g., encoding a rhabdoviral G glycoprotein or functional fragment or derivative thereof, or recombinant fusion protein thereof, as described herein, and “helper constructs” comprise proteins useful for the formation of, e.g., virions. Such helper constructs can encode, e.g., structural proteins, and can be packaging vectors that are, e.g., plasmids.

In aspects, the retroviral vector expression system comprises, consists essentially of, or consists of a nucleotide sequence encoding a chimeric antigen receptor (CAR) comprising, consisting essentially of, or consisting of an antigen binding domain, a transmembrane domain, and an intracellular signaling domain. In aspects, the retroviral vector expression system comprises, consists essentially of, or consists of a nucleotide sequence encoding a transgene.

In aspects, the present disclosure provides a method of making a membraned vesicle, an enveloped viral particle, an enveloped delivery vehicle, or a recombinant viral vector, the method comprising, consisting essentially of, or consisting of: a) transfecting or transducing a packaging host cell with a retroviral vector expression system as described herein; and b) recovering the membraned vesicle, enveloped viral particle, or recombinant viral vector produced by the transfected or transduced packaging host cell.

In aspects, the present disclosure provides a method of making a membraned vesicle, an enveloped viral particle, an enveloped delivery vehicle, or a recombinant viral vector, the method comprising, consisting essentially of, or consisting of: a) transfecting or transducing a first host cell with a first vector expression system comprising, consisting essentially of, or consisting of one or more nucleotide sequences encoding a recombinant fusion protein as described herein, wherein the first host cell is a packaging host cell; b) transfecting or transducing a second host cell with a second vector expression system comprising, consisting essentially of, or consisting of one or more nucleotide sequences encoding a second recombinant fusion protein as described herein; c) collecting supernatant from the transfected or transduced second host cell; d) administering the supernatant to the transfected or transduced first host cell; and e) recovering the membraned vesicle, enveloped delivery vehicle, enveloped viral particle, or recombinant viral vector produced by the transfected or transduced first host cell after administration of supernatant produced by the transfected or transduced second host cell.

“Transfecting” and “transfection” as used herein means non-viral means of introducing nucleic acid into a cell. “Transducing” and “transduction” as used herein means use of viral means of introducing nucleic acid into a cell.

In aspects, any method of making an enveloped delivery vehicle, lentivirus, retroviral vector expression system, recombinant viral vector, etc. as described herein can comprise transduction in a medium comprising a poloxamer-based chemical adjuvant. In aspects, the poloxamer-based chemical adjuvant is selected from vectofusin-1, poloxamer F108, and Lentiboost™. In aspects, the method of making comprises a spinoculation step at the beginning of transduction.

In aspects, the packaging host cell is HEK 293T or a 293-cell suspension line.

In aspects, the present disclosure provides a plasmid comprising, consisting essentially of, or consisting of one or more nucleotide sequences encoding a recombinant fusion protein as described herein or a recombinant protein system as described herein.

In aspects, the present disclosure provides a composition as described herein or a retroviral vector expression system as described herein for use in treating a disease in a mammal.

The mammal may be any suitable mammal. Mammals include, but are not limited to, the order Rodentia, such as mice, and the order Lagomorpha, such as rabbits. The mammal can be from the order Carnivora, including Felines (cats) and Canines (dogs). The mammal can be from the order Artiodactyla, including Bovines (cows) and Swines (pigs) or of the order Perissodactyla, including Equines (horses). The mammal can be of the order Primates, Cebids, or Simioids (monkeys) or of the order Anthropoids (humans and apes). In aspects, the mammal is human.

In aspects the disease is a genetic disease. In aspects the disease is a cancer. In aspects the disease is beta-thalassemia, anemia, sickle cell disease, Gaucher disease, Parkinson disease, immunologic deficiency syndromes, mucopolysaccharidosis III, severe combined immunodeficiency disease, hepatitis C, chronic hepatitis C, pancytopenia, X-linked combined immunodeficiency diseases, epidermolysis bullosa, solid tumors, melanoma, Fanconi anemia, adenosine deaminase deficiency, granulomatous disease, chronic cystic fibrosis, HIV infections, HIV seropositivity, Hodgkin's lymphoma, Bruton type agammaglobulinemia, Hemophilia A, arthritis, or choroidal neovascularization. In other aspects, the disease is any disease or condition that can be treated by delivery of a gene editing system.

As used herein, “treatment” or “treat” refers to a therapeutic intervention that ameliorates a sign or symptom of a disease or pathological condition after it has begun to develop, and also includes addressing a medical condition or disease with the objective of improving or stabilizing an outcome in a mammal being treated. “Treating,” “treat,” and “treatment” have grammatically corresponding meanings. In the context of cancer, the terms “treat” or “treating” or “treatment” can, among other things, refer to inducing apoptosis of cancerous cells, reducing the size of a cancerous tumor, delaying the growth of tumors, or inducing or enhancing an immune response against one or more cancerous cells, where the immune response has the effect of inducing apoptosis, reducing the size of a tumor, or the like. The terms “treat,” “treating,” “treatment,” “therapeutically effective,” “prevention,” etc. used herein do not necessarily imply 100% or complete treatment/prevention/etc. Rather, there are varying degrees, which one of ordinary skill in the art recognizes as having a potential benefit or therapeutic effect. In this respect, the membraned vesicles, enveloped viral particles, recombinant viral vectors, etc. as described herein and methods as described herein can provide any amount of any level of treatment. Furthermore, the treatment provided by the disclosed method can include the treatment of one or more conditions or symptoms of the disease or condition being treated.

In aspects, a method of treating comprises administering a composition as described herein or a retroviral vector expression system as described herein, comprising a rhabdoviral G glycoprotein or functional fragment or derivative thereof or recombinant fusion protein thereof, and subsequently administering a composition as described herein or a retroviral vector expression system as described herein, comprising a different rhabdoviral G glycoprotein or functional fragment or derivative thereof or recombinant fusion protein thereof. The subsequent administration may be within hours, days, months, or years of the first administration, with scheduling to be determined by patient needs. Multiple rounds of subsequent administrations may be performed, using the same or different rhabdoviral G glycoproteins as determined by the patient needs. Without wishing to be bound by theory, it is believed that administering a different rhabdoviral G glycoprotein or functional fragment or derivative thereof reduces the likelihood of decreased effectiveness due to any immune response that is directed against the first rhabdoviral G glycoprotein or functional fragment or derivative thereof. A rhabdoviral G glycoprotein or functional fragment or derivative thereof or recombinant fusion protein thereof described herein can be the first administered, and a rhabdoviral G glycoprotein or functional fragment or derivative thereof or recombinant fusion protein thereof as described herein that has a different rhabdoviral G glycoprotein than the first rhabdoviral G glycoprotein or functional fragment or derivative thereof or recombinant fusion protein thereof can be the subsequently administered rhabdoviral G glycoprotein or functional fragment or derivative thereof or recombinant fusion protein thereof. This sequence of events can be termed “re-dosing”.

In aspects, the composition is administered intravenously. In aspects, the composition is administered intraperitoneally.

Rhabdoviral G glycoproteins can associate into trimers at the surface of a native virus, an enveloped delivery vehicle as described herein, a membraned vesicle as described herein, an enveloped viral particle as described herein, or a recombinant viral vector as described herein. As used herein, a “mixed rhabdoviral G glycoprotein trimer” comprises, consists essentially of, or consists of three rhabdoviral G glycoproteins or functional fragments or derivatives thereof (wherein each is optionally within a recombinant fusion protein), wherein (a) at least one of the rhabdoviral G glycoproteins or functional fragments or derivatives thereof in the trimer is within a recombinant fusion protein comprising, consisting essentially of, or consisting of a rhabdoviral G glycoprotein or a functional fragment or derivative thereof engineered to reduce or abolish natural receptor binding specificity and a polypeptide (polypeptide tag) that is covalently bound to a polypeptide (polypeptide catcher) of a second recombinant fusion protein, and (b) at least one of the rhabdoviral G glycoproteins or functional fragments or derivatives thereof in the trimer is (1) not within a recombinant fusion protein and does not have a polypeptide tag or (2) within a recombinant fusion protein comprising, consisting essentially of, or consisting of a rhabdoviral G glycoprotein or a functional fragment or derivative thereof engineered to reduce or abolish natural receptor binding specificity and a polypeptide (polypeptide tag) that is covalently bound to a polypeptide (polypeptide catcher) of a third recombinant fusion protein, wherein the second and third recombinant fusion proteins are not the same (e.g., the portions of the second and third recombinant fusion proteins that are not the polypeptide catcher have different amino acid sequences). For example, a mixed rhabdoviral G glycoprotein trimer can include (a) two rhabdoviral G glycoproteins or functional fragments or derivatives thereof engineered to reduce or abolish natural receptor binding specificity, wherein the two rhabdoviral G glycoproteins or functional fragments or derivatives are not within recombinant fusion proteins and do not have polypeptide tags, and (b) a rhabdoviral G glycoprotein that is within a recombinant fusion protein comprising, consisting essentially of, or consisting of a rhabdoviral G glycoprotein or a functional fragment or derivative thereof engineered to reduce or abolish natural receptor binding specificity and a polypeptide (polypeptide tag) that is covalently bound to a polypeptide (polypeptide catcher) of a second recombinant fusion protein. As another example, a mixed rhabdoviral G glycoprotein trimer can include (a) one rhabdoviral G glycoprotein or a functional fragment or derivative thereof engineered to reduce or abolish natural receptor binding specificity, wherein the one rhabdoviral G glycoprotein or functional fragment or derivative is not within a recombinant fusion protein and does not have a polypeptide tag, and (b) two rhabdoviral G glycoproteins that each are within separate recombinant fusion proteins comprising, consisting essentially of, or consisting of a rhabdoviral G glycoprotein or a functional fragment or derivative thereof, engineered to reduce or abolish natural receptor binding specificity, and a polypeptide (polypeptide tag) that is covalently bound to a polypeptide (polypeptide catcher) within separate second recombinant fusion proteins, wherein the separate second recombinant fusion proteins are the same or not the same (e.g., the portions of the separate second recombinant fusion proteins that are not the polypeptide catcher are the same or different amino acid sequences). As another example, a mixed rhabdoviral G glycoprotein trimer can include three rhabdoviral G glycoproteins that each are within separate recombinant fusion proteins comprising, consisting essentially of, or consisting of a rhabdoviral G glycoprotein or a functional fragment or derivative thereof, engineered to reduce or abolish natural receptor binding specificity, and a polypeptide (polypeptide tag), wherein at least one of the polypeptide tags is not covalently bound to a polypeptide (polypeptide catcher) and at least one of the polypeptide tags is covalently bound to a polypeptide (polypeptide catcher) within a second recombinant fusion protein. As another example, a mixed rhabdoviral G glycoprotein trimer can include three rhabdoviral G glycoproteins that each are within separate recombinant fusion proteins comprising, consisting essentially of, or consisting of a rhabdoviral G glycoprotein or a functional fragment or derivative thereof, engineered to reduce or abolish natural receptor binding specificity, and a polypeptide (polypeptide tag) that is covalently bound to a polypeptide (polypeptide catcher) within separate second recombinant fusion proteins, wherein at least one of the separate second recombinant fusion proteins is not the same as another one of the separate second recombinant fusion proteins (e.g., the portions of the separate second recombinant fusion proteins that are not the polypeptide catcher have among them at least two different amino acid sequences).

Trimers of rhabdoviral G glycoproteins or functional fragments or derivatives thereof can be mixed trimers based on the identity of the amino acid sequence of the polypeptide fused to polypeptide catcher. Trimers of rhabdoviral G glycoproteins or functional fragments or derivatives thereof can be mixed trimers based on the presence of polypeptide fused to polypeptide catcher for one or two rhabdoviral G glycoproteins or functional fragments or derivatives thereof and absence of polypeptide fused to polypeptide catcher for the other two or one rhabdoviral G glycoprotein(s) or functional fragment(s) or derivative(s) thereof, wherein the other two or one rhabdoviral G glycoprotein(s) or functional fragment(s) or derivative(s) thereof may or may not be within recombinant fusion proteins that comprise a polypeptide tag. Trimers of rhabdoviral G glycoproteins or functional fragments or derivatives thereof can be mixed trimers based on the presence of polypeptide fused to polypeptide catcher, wherein at least two of the amino acid sequences of the polypeptides fused to polypeptide catcher are different.

Trimers of rhabdoviral G glycoproteins or functional fragments or derivatives thereof, wherein the rhabdoviral G glycoproteins or functional fragments or derivatives thereof are within recombinant fusion proteins comprising a polypeptide (polypeptide tag) may have one, two, or all three polypeptide tags separately covalently bound to polypeptides (polypeptide catchers) within separate second recombinant fusion proteins (regardless of the identity of the portions of the separate second recombinant fusion proteins that are not the polypeptide catcher). The number of bound polypeptide catchers to the trimer is the occupancy of the trimer, wherein the trimer may have full occupancy (polypeptide catchers covalently bound to all polypeptide tags of the trimer), partial occupancy (polypeptide catchers covalently bound to fewer than all polypeptide tags of the trimer, e.g., two or one polypeptide tags), or no occupancy (no polypeptide catchers covalently bound to any polypeptide tags of the trimer).

In aspects, the present disclosure provides a method of making a mixed rhabdoviral G glycoprotein trimer, the method comprising, consisting essentially of, or consisting of: a) transfecting or transducing a packaging host cell with a retroviral vector expression system as described herein; and b) recovering the mixed rhabdoviral G glycoprotein trimer.

In aspects, the present disclosure provides a method of making a mixed rhabdoviral G glycoprotein trimer, the method comprising, consisting essentially of, or consisting of: a) transfecting or transducing a first host cell with a first vector expression system comprising, consisting essentially of, or consisting of one or more nucleotide sequences encoding a recombinant fusion protein as described herein, wherein the first host cell is a packaging host cell; b) transfecting or transducing a second host cell with a second vector expression system comprising, consisting essentially of, or consisting of one or more nucleotide sequences encoding the second recombinant fusion protein; c) contacting virus produced by the first host cell with second recombinant fusion protein produced by the second host cell; and d) obtaining virus decorated with the second recombinant fusion protein, wherein the virus comprises a mixed trimer, wherein the mixed trimer comprises (a) at least one recombinant fusion protein, wherein the polypeptide tag of the recombinant fusion protein is covalently bound to the polypeptide catcher of the second recombinant fusion protein, and (b) at least one (i) recombinant fusion protein, wherein the polypeptide tag of the recombinant fusion protein is not covalently bound to a polypeptide catcher or (ii) rhabdoviral G glycoprotein or a functional fragment or derivative thereof that is not within a recombinant fusion protein.

Mixed trimers may be prepared using any suitable method. For example, a recombinant fusion protein comprising, consisting essentially of, or consisting of (a) a rhabdoviral G glycoprotein or a functional fragment or derivative thereof and (b) a polypeptide (polypeptide tag) that is capable of forming a covalent bond with an amino acid residue in a second recombinant fusion protein, wherein the polypeptide tag is N-terminal to the rhabdoviral G glycoprotein, can be co-expressed with the second recombinant fusion protein, wherein the second recombinant fusion protein comprises a polypeptide (polypeptide catcher) that is capable of forming a covalent bond with an amino acid residue in the polypeptide tag. In such a method, collection of a membraned vesicle, an enveloped viral particle, an enveloped delivery vehicle, a recombinant viral vector, etc. will include at least some having polypeptide tag covalently bonded to polypeptide catcher. In such a method, multiple, different second recombinant fusion proteins can be co-expressed to provide a membraned vesicle, an enveloped viral particle, an enveloped delivery vehicle, a recombinant viral vector, etc. having rhabdoviral G glycoproteins or functional fragments or derivatives thereof having different second recombinant fusion proteins covalently bonded. As another example, a membraned vesicle, an enveloped viral particle, an enveloped delivery vehicle, a recombinant viral vector, etc. can be prepared having a recombinant fusion protein comprising, consisting essentially of, or consisting of (a) a rhabdoviral G glycoprotein or a functional fragment or derivative thereof and (b) a polypeptide (polypeptide tag) that is capable of forming a covalent bond with an amino acid residue in a second recombinant fusion protein, wherein the polypeptide tag is N-terminal to the rhabdoviral G glycoprotein, without co-expressing with the second recombinant fusion protein. Then, a membraned vesicle, an enveloped viral particle, an enveloped delivery vehicle, a recombinant viral vector, etc. can be collected, e.g., isolated and/or purified, without having the polypeptide catcher bound. One or more second recombinant fusion proteins can then be covalently bound to at least some of the polypeptide tags of the recombinant fusion proteins, wherein more than one second recombinant fusion proteins can be different recombinant fusion proteins. Different second recombinant fusion proteins can be covalently bound sequentially or simultaneously, and can be bound in predetermined ratios.

In aspects, the present disclosure provides a method of reducing inhibition of rhabdoviral G glycoprotein-mediated fusion in serum, wherein the inhibition is by complement inactivation or competitive binding with LDL or vLDL (e.g., ApoB-100-containing lipoproteins), the method comprising, consisting essentially of, or consisting of covalently bonding to a rhabdoviral G glycoprotein or functional fragment or derivative thereof a targeting molecule, and exposing the rhabdoviral G glycoprotein or functional fragment or derivative thereof with targeting molecule to serum, wherein complement inactivation or competitive binding with LDL or vLDL is inhibited. In aspects, the present disclosure provides a method of reducing inhibition of rhabdoviral G glycoprotein-mediated fusion in serum, wherein the inhibition is by complement inactivation or competitive binding with LDL or vLDL, the method comprising, consisting essentially of, or consisting of producing the rhabdoviral G glycoprotein or functional fragment or derivative thereof as the rhabdoviral G glycoprotein or functional fragment or derivative thereof within a recombinant fusion protein as described herein, and exposing the rhabdoviral G glycoprotein or functional fragment or derivative thereof to serum, wherein complement inactivation or competitive binding with LDL or vLDL is inhibited. Reduction in inhibition can be of any amount, e.g., reduced by 90%, 80%, 70%, 60%, 50%, 40%, 30%, 20%, 10%, 5%, 2%, 1%, or any range between these percentages. In aspects, producing vectors as described herein in a cell that expresses CD55 (or overexpresses CD55) can increase resistance to complement inactivation.

Methods contemplated include use of a lentivirus as described herein to genetically modify hematopoietic stem cells for use in treatment of a disease in a mammal. The modification can be to treat, e.g., transfusion-dependent β-thalassemia, cerebral adrenoleukodystrophy, sickle cell disease, metachromatic leukodystrophy and adrenoleukodystrophy, Wiskott-Aldrich syndrome, X-SCID or ADA-SCID (ADA: adenosine deaminase, SCID: severe combined immunodeficiency), Fanconi anemia, or X-linked chronic granulomatous disease.

Commonly owned International Patent application Ser. No. ______, titled “CD3-REDIRECTED VECTORS, COMPONENTS THEREOF, AND USES OF THE VECTORS AND COMPONENTS THEREOF,” filed on Apr. 12, 2024 and having Attorney Docket No. 770399, is incorporated herein by reference in its entirety.

International Patent Application No. PCT/US2024/013058, titled “MODIFIED RHABDOVIRUS GLYCOPROTEINS AND USES THEREOF,” filed on Jan. 26, 2024, is incorporated herein by reference in its entirety.

The following are certain aspects of the disclosure.
1. A recombinant fusion protein comprising, consisting essentially of, or consisting of (a) a rhabdoviral G glycoprotein or a functional fragment or derivative thereof and (b) a polypeptide (polypeptide tag) that is capable of forming a covalent bond with an amino acid residue in a second recombinant fusion protein, wherein the polypeptide tag is N-terminal to the rhabdoviral G glycoprotein, and wherein the second recombinant fusion protein comprises a polypeptide (polypeptide catcher) that is capable of forming a covalent bond with an amino acid residue in the polypeptide tag.
2. The recombinant fusion protein of aspect 1, polypeptide tag is SpyTag (SEQ ID NO: 1), SpyTag002 (SEQ ID NO: 2), SpyTag003 (SEQ ID NO: 3), Isopeptag (SEQ ID NO: 4), or SnoopTag (SEQ ID NO: 5).
3. The recombinant fusion protein of aspect 2, wherein the polypeptide tag is SpyTag (SEQ ID NO: 1).
4. The recombinant fusion protein of any one of aspects 1-3, wherein the fusion protein comprises a linker between the polypeptide tag and the rhabdoviral G glycoprotein or functional fragment or derivative thereof.
5. The recombinant fusion protein of aspect 4, wherein the linker is flexible.
6. The recombinant fusion protein of aspect 5, wherein the linker is

(SEQ ID NO: 130)
AAASGGSGGGGSGGGGSGP,
(SEQ ID NO: 131)
AAASGGSGGGGSGGGGS,
(SEQ ID NO: 132)
GGGGSGGGGSGGGGSGGGGS,
(SEQ ID NO: 36)
GGGGSGGGGSGGGGS,
(SEQ ID NO: 133)
GGGGSGGGGS,
(SEQ ID NO: 134)
GGGGS,
(SEQ ID NO: 135)
GGGGGGGG,
(SEQ ID NO: 136)
GGGGGG,
(SEQ ID NO: 137)
GSAGSAAGSGEF,
and
(SEQ ID NO: 138)
VPGVGVPGVG).

7. The recombinant fusion protein of aspect 4, wherein the linker is rigid.
8. The recombinant fusion protein of aspect 7, wherein the linker is

(SEQ ID NO: 139)
PAPAP,
(SEQ ID NO: 140)
EAAAKEAAAKEAAAK,
(SEQ ID NO: 141)
EAAAKEAAAK,
(SEQ ID NO: 142)
EAAAK,
(SEQ ID NO: 143)
AEAAAKEAAAKEAAAKEAAAKALEAEAAAKEAAAKEAAAKEAAAKA,
(SEQ ID NO: 144)
AEAAAKEAAAKA,
(SEQ ID NO: 145)
ESKYGPPCPPCP,
(SEQ ID NO: 146)
CPPCPAPELLGGPSVF,

and
   alanine-proline (AP) repeated for a total of 10 to 34 amino acids (SEQ ID NO: 147).
9. The recombinant fusion protein of any one of aspects 1-8, wherein the rhabdoviral G glycoprotein or functional fragment or derivative thereof is of a Flanders virus glycoprotein (FLAV-G) (SEQ ID NO: 13), a Chandipura virus glycoprotein (CHPV-G) (SEQ ID NO: 14), a Perinet virus glycoprotein (PERV-G) (SEQ ID NO: 15), a Piry virus glycoprotein (PIRYV-G) (SEQ ID NO: 16), a Fukuoka virus glycoprotein (FUKV-G) (SEQ ID NO: 17), a Joinjakaka virus glycoprotein (JOIV-G) (SEQ ID NO: 18), a Kumasi virus glycoprotein (KRV-G) (SEQ ID NO: 19), a Keuraliba virus glycoprotein (KEUV-G) (SEQ ID NO: 20), an Isfahan glycoprotein (ISFV-G), a Jurona glycoprotein (JURV-G), a Mediterranean Bat glycoprotein (MBV-G), a Malpais Spring glycoprotein (MSPV-G), a Radi glycoprotein (RADV-G), a Rhinolophus affinis-G, a Yug Bugdanavoc glycoprotein (YBV-G), a Yinshui Bat glycoprotein (YSBV-G), a Kimberley glycoprotein (KIMV-G), a Kanyawara glycoprotein (KYAV-G), a La Joya glycoprotein (LJV-G), a Mosquiero glycoprotein (MQOV-G), a Parry Creek glycoprotein (PCV-G), a Bas Congo glycoprotein (BASV-G), a Bovine Ephemeral fever glycoprotein (BEFV-G), a Curionopolis glycoprotein (CURV-G), a Drosophila melanogaster sigmavirus glycoprotein (DMelSV-G), a Niakha glycoprotein (NIAV-G), a Puerto almandras glycoprotein (PTAMV-G), or a Tupaia rhabdovirus (TUPTV-G).
10. The recombinant fusion protein of any one of aspects 1-8, wherein the rhabdoviral G glycoprotein or functional fragment or derivative thereof is of Vesiculovirus glycoprotein or a functional fragment or derivative thereof.
11. The recombinant fusion protein of any one of aspects 1-8, wherein the rhabdoviral G glycoprotein or functional fragment or derivative thereof is of Vesiculovirus indiana, Vesiculovirus newjersey, Vesiculovirus carajas, or Vesiculovirus alagoas.
12. The recombinant fusion protein of aspect 11, wherein the rhabdoviral G glycoprotein or functional fragment or derivative thereof is of Vesiculovirus indiana (SEQ ID NO 21).
13. The recombinant fusion protein of aspect 11, wherein the rhabdoviral G glycoprotein or functional fragment or derivative thereof is of Vesiculovirus newjersey (SEQ ID NO: 22).
14. The recombinant fusion protein of any one of aspects 1-13, wherein the rhabdoviral G glycoprotein is substantially intact.
15. The recombinant fusion protein of any one of aspects 1-13, wherein the rhabdoviral G glycoprotein is a functional fragment or derivative thereof.
16. The recombinant fusion protein of aspect 15, wherein the cytoplasmic tail of the glycoprotein is truncated, deleted, or replaced with another sequence.
17. The recombinant fusion protein of any one of aspects 1-16, wherein the rhabdoviral G glycoprotein is engineered to reduce or abolish its natural receptor binding specificity.
18. The recombinant fusion protein of aspect 17, wherein the rhabdoviral G glycoprotein is engineered to have a mutation to reduce or abolish its natural receptor binding specificity.
19. The recombinant fusion protein of aspect 18, wherein the rhabdoviral G glycoprotein comprises a mutation at one or more positions corresponding to H8, K47, Y209, and K354 on the Vesiculovirus indiana glycoprotein (SEQ ID NO: 21)
20. The recombinant fusion protein of aspect 18 or 19, wherein the mutation is a substitution.
21. The recombinant fusion protein of aspect 20, wherein the substitution is with a Q.
22. The recombinant fusion protein of aspect 20 or 21, wherein the mutation is a substitution at three or more positions corresponding to H8, K47, Y209, and K354 on the Vesiculovirus indiana glycoprotein (SEQ ID NO: 21)
23. The recombinant fusion protein of aspect 18 or 19, wherein the mutation is a deletion.
24. The recombinant fusion protein of aspect 23, wherein the mutation is a single deletion at K47.
25. The recombinant fusion protein of any one of aspects 1-24, wherein the polypeptide tag is covalently bonded to the polypeptide catcher.
26. The recombinant fusion protein of aspect 25, wherein the formation of the covalent bond is spontaneous.
27. The recombinant fusion protein of aspect 25 or 26, wherein the covalent bond is an isopeptide bond.
28. The recombinant fusion protein of any one of aspects 25-27, wherein the polypeptide tag and the polypeptide catcher are the pair SpyTag and SpyCatcher (SEQ ID NO: 1 and SEQ ID NO: 30), SpyTag002 and SpyCatcher002 (SEQ ID NO: 2 and SEQ ID NO: 31), SpyTag003 and SpyCatcher003 (SEQ ID NO: 3 and SEQ ID NO: 32), SpyTag and KTag (SEQ ID NO: 1 and SEQ ID NO: 33), Isopeptag and pilin-C(SEQ ID NO: 4 and SEQ ID NO: 34), or SnoopTag and SnoopCatcher (SEQ ID NO: 5 and SEQ ID NO: 35).
29. The recombinant fusion protein of any one of aspects 25-28, wherein the second recombinant fusion protein comprises a targeting molecule.
30. The recombinant fusion protein of aspect 29, wherein the targeting molecule is a ligand.
31. The recombinant fusion protein of aspect 30, wherein the ligand is epidermal growth factor (EGF), a mutant EGF (EFGm123) (SEQ ID NO: 59), stem cell factor (SCF) (SEQ ID NO: 52), thrombopoietin (TPO) (SEQ ID NO: 55), human hepatocyte growth factor (HGF) (SEQ ID NO: 56), or type 1 insulin-like growth factor (IGF1) (SEQ ID NO: 58).
32. The recombinant fusion protein of aspect 29, wherein the targeting molecule is a single chain variable fragment (scFv).
33. The recombinant fusion protein of aspect 32, wherein the scFv binds epidermal growth factor (EGF), human epidermal growth factor receptor 2 (Her2), cluster of differentiation 3 (CD3), or cluster of differentiation 117 (cKit), mucin-16 (MUC16), B cell maturation antigen (BCMA), or Nectin4.
34. The recombinant fusion protein of any one of aspects 29-33, wherein the second recombinant fusion protein comprises a linker between the targeting molecule and the polypeptide catcher.
35. The recombinant fusion protein of aspect 34, wherein the linker is flexible.
36. The recombinant fusion protein of aspect 35, wherein the linker is

(SEQ ID NO: 130)
AAASGGSGGGGSGGGGSGP,
(SEQ ID NO: 131)
AAASGGSGGGGSGGGGS,
(SEQ ID NO: 132)
GGGGSGGGGSGGGGSGGGGS,
(SEQ ID NO: 36)
GGGGSGGGGSGGGGS,
(SEQ ID NO: 133)
GGGGSGGGGS,
(SEQ ID NO: 134)
GGGGS,
(SEQ ID NO: 135)
GGGGGGGG,
(SEQ ID NO: 136)
GGGGGG,
(SEQ ID NO: 137)
GSAGSAAGSGEF,
and
(SEQ ID NO: 138)
VPGVGVPGVG).

37. The recombinant fusion protein of aspect 34, wherein the linker is rigid.
38. The recombinant fusion protein of aspect 37, wherein the linker is

(SEQ ID NO: 139)
PAPAP,
(SEQ ID NO: 140)
EAAAKEAAAKEAAAK,
(SEQ ID NO: 141)
EAAAKEAAAK,
(SEQ ID NO: 142)
EAAAK,
(SEQ ID NO: 143)
AEAAAKEAAAKEAAAKEAAAKALEAEAAAKEAAAKEAAAKEAAAKA,
(SEQ ID NO: 144)
AEAAAKEAAAKA,
(SEQ ID NO: 145)
ESKYGPPCPPCP,
(SEQ ID NO: 146)
CPPCPAPELLGGPSVF,

and
   alanine-proline (AP) repeated for a total of 10 to 34 amino acids (SEQ ID NO: 147).
39. The recombinant fusion protein of any one of aspects 29-38, wherein the targeting molecule is N terminal to the polypeptide catcher.
40. The recombinant fusion protein of any one of aspects 29-38, wherein the targeting molecule is C terminal to the polypeptide catcher.
41. The recombinant fusion protein of any one of aspects 25-40, wherein the second recombinant fusion protein comprises a signal peptide.
42. The recombinant fusion protein of any one of aspects 25-41, wherein the second recombinant fusion protein comprises a tag for western detection or purification.
43. The recombinant fusion protein of aspect 42, wherein the tag is a tag for western detection.
44. The recombinant fusion protein of aspect 42, wherein the tag is a tag for purification.
45. A recombinant protein system comprising, consisting essentially of, or consisting of (a) a recombinant fusion protein comprising, consisting essentially of, or consisting of (1) a rhabdoviral G glycoprotein or a functional fragment or derivative thereof and (2) a polypeptide (polypeptide tag) that is capable of forming a covalent bond with an amino acid residue in a second recombinant fusion protein, wherein the polypeptide tag is N-terminal to the rhabdoviral G glycoprotein, and (b) the second recombinant fusion protein, wherein the second recombinant fusion protein comprises a polypeptide (polypeptide catcher) that is capable of forming a covalent bond with an amino acid residue in the polypeptide tag.
46. The recombinant protein system of aspect 45, wherein the polypeptide tag and the polypeptide catcher are the pair SpyTag and SpyCatcher (SEQ ID NO: 1 and SEQ ID NO: 30), SpyTag002 and SpyCatcher002 (SEQ ID NO: 2 and SEQ ID NO: 31), SpyTag003 and SpyCatcher003 (SEQ ID NO: 3 and SEQ ID NO: 32), SpyTag and KTag (SEQ ID NO: 1 and SEQ ID NO: 33), Isopeptag and pilin-C(SEQ ID NO: 4 and SEQ ID NO: 34), or SnoopTag and SnoopCatcher (SEQ ID NO: 5 and SEQ ID NO: 35).
47. The recombinant protein system of aspect 46, wherein the polypeptide tag and the polypeptide catcher are the pair SpyTag003 and SpyCatcher003 (SEQ ID NO: 3 and SEQ ID NO: 32).
48. The recombinant protein system of any one of aspects 45-47, wherein the fusion protein comprises a linker between the polypeptide tag and the rhabdoviral G glycoprotein or functional fragment or derivative thereof.
49. The recombinant protein system of aspect 48, wherein the linker is flexible.
50. The recombinant protein system of aspect 49, wherein the linker is

(SEQ ID NO: 130)
AAASGGSGGGGSGGGGSGP,
(SEQ ID NO: 131)
AAASGGSGGGGSGGGGS,
(SEQ ID NO: 132)
GGGGSGGGGSGGGGSGGGGS,
(SEQ ID NO: 36)
GGGGSGGGGSGGGGS,
(SEQ ID NO: 133)
GGGGSGGGGS,
(SEQ ID NO: 134)
GGGGS,
(SEQ ID NO: 135)
GGGGGGGG,
(SEQ ID NO: 136)
GGGGGG,
(SEQ ID NO: 137)
GSAGSAAGSGEF,
and
(SEQ ID NO: 138)
VPGVGVPGVG).

51. The recombinant protein system of aspect 48, wherein the linker is rigid.
52. The recombinant protein system of aspect 51, wherein the linker is

(SEQ ID NO: 139)
PAPAP,
(SEQ ID NO: 140)
EAAAKEAAAKEAAAK,
(SEQ ID NO: 141)
EAAAKEAAAK,
(SEQ ID NO: 142)
EAAAK,
(SEQ ID NO: 143)
AEAAAKEAAAKEAAAKEAAAKALEAEAAAKEAAAKEAAAKEAAAKA,
(SEQ ID NO: 144)
AEAAAKEAAAKA,
(SEQ ID NO: 145)
ESKYGPPCPPCP,
(SEQ ID NO: 146)
CPPCPAPELLGGPSVF,

and
   alanine-proline (AP) repeated for a total of 10 to 34 amino acids (SEQ ID NO: 147).
53. The recombinant protein system of any one of aspects 45-52, wherein the rhabdoviral G glycoprotein or functional fragment or derivative thereof is of a Flanders virus glycoprotein (FLAV-G) (SEQ ID NO: 13), a Chandipura virus glycoprotein (CHPV-G) (SEQ ID NO: 14), a Perinet virus glycoprotein (PERV-G) (SEQ ID NO: 15), a Piry virus glycoprotein (PIRYV-G) (SEQ ID NO: 16), a Fukuoka virus glycoprotein (FUKV-G) (SEQ ID NO: 17), a Joinjakaka virus glycoprotein (JOIV-G) (SEQ ID NO: 18), a Kumasi virus glycoprotein (KRV-G) (SEQ ID NO: 19), a Keuraliba virus glycoprotein (KEUV-G) (SEQ ID NO: 20), an Isfahan glycoprotein (ISFV-G), a Jurona glycoprotein (JURV-G), a Mediterranean Bat glycoprotein (MBV-G), a Malpais Spring glycoprotein (MSPV-G), a Radi glycoprotein (RADV-G), a Rhinolophus affinis-G, a Yug Bugdanavoc glycoprotein (YBV-G), a Yinshui Bat glycoprotein (YSBV-G), a Kimberley glycoprotein (KIMV-G), a Kanyawara glycoprotein (KYAV-G), a La Joya glycoprotein (LJV-G), a Mosquiero glycoprotein (MQOV-G), a Parry Creek glycoprotein (PCV-G), a Bas Congo glycoprotein (BASV-G), a Bovine Ephemeral fever glycoprotein (BEFV-G), a Curionopolis glycoprotein (CURV-G), a Drosophila melanogaster sigmavirus glycoprotein (DMelSV-G), a Niakha glycoprotein (NIAV-G), a Puerto almandras glycoprotein (PTAMV-G), or a Tupaia rhabdovirus (TUPTV-G).
54. The recombinant protein system of any one of aspects 45-52, wherein the rhabdoviral G glycoprotein or functional fragment or derivative thereof is of a Vesiculovirus glycoprotein or a functional fragment or derivative thereof.
55. The recombinant protein system of any one of aspects 45-52, wherein the rhabdoviral G glycoprotein or functional fragment or derivative thereof is of Vesiculovirus indiana, Vesiculovirus newjersey, Vesiculovirus carajas, or Vesiculovirus alagoas.
56. The recombinant protein system of aspect 55, wherein the rhabdoviral G glycoprotein or functional fragment or derivative thereof is of Vesiculovirus indiana (SEQ ID NO: 21).
57. The recombinant protein system of aspect 55, wherein the rhabdoviral G glycoprotein or functional fragment or derivative thereof is of Vesiculovirus newjersey (SEQ ID NO: 22).
58. The recombinant protein system of any one of aspects 45-57, wherein the rhabdoviral G glycoprotein is substantially intact.
59. The recombinant protein system of any one of aspects 45-57, wherein the rhabdoviral G glycoprotein is a functional fragment or derivative thereof.
60. The recombinant protein system of aspect 59, wherein the cytoplasmic tail of the glycoprotein is truncated, deleted, or replaced with another sequence.
61. The recombinant protein system of any one of aspects 45-60, wherein the rhabdoviral G glycoprotein is engineered to reduce or abolish its natural receptor binding specificity.
62. The recombinant protein system of aspect 61, wherein the rhabdoviral G glycoprotein is engineered to have a mutation to reduce or abolish its natural receptor binding specificity.
63. The recombinant protein system of aspect 62, wherein the rhabdoviral G glycoprotein comprises a mutation at one or more positions corresponding to H8, K47, Y209, and K354 on the Vesiculovirus indiana glycoprotein (SEQ ID NO: 21).
64. The recombinant protein system of aspect 62 or 63, wherein the mutation is a substitution.
65. The recombinant protein system of aspect 64, wherein the substitution is with a Q.
66. The recombinant protein system of aspect 64 or 65, wherein the mutation is a substitution at three or more positions corresponding to H8, K47, Y209, and K354 on the Vesiculovirus indiana glycoprotein (SEQ ID NO: 21)
67. The recombinant protein system of aspect 62 or 63, wherein the mutation is a deletion.
68. The recombinant protein system of aspect 67, wherein the mutation is a single deletion at K47.
69. The recombinant protein system of any one of aspects 45-68, wherein the second recombinant fusion protein comprises a targeting molecule.
70. The recombinant protein system of aspect 69, wherein the targeting molecule is a ligand.
71. The recombinant protein system of aspect 70, wherein the ligand is epidermal growth factor (EGF), a mutant EGF (EFGm123) (SEQ ID NO: 59), stem cell factor (SCF) (SEQ ID NO: 52), thrombopoietin (TPO) (SEQ ID NO: 55), human hepatocyte growth factor (HGF) (SEQ ID NO: 56), or type 1 insulin-like growth factor (IGF1) (SEQ ID NO: 58).
72. The recombinant protein system of aspect 69, wherein the targeting molecule is a single chain variable fragment (scFv).
73. The recombinant protein system of aspect 72, wherein the scFv binds epidermal growth factor (EGF), human epidermal growth factor receptor 2 (Her2), cluster of differentiation 3 (CD3), or cluster of differentiation 117 (cKit), mucin-16 (MUC16), B cell maturation antigen (BCMA), or Nectin4.
74. The recombinant protein system of any one of aspects 69-73, wherein the second recombinant fusion protein comprises a linker between the targeting molecule and the polypeptide catcher.
75. The recombinant protein system of aspect 74, wherein the linker is flexible.
76. The recombinant protein system of aspect 75, wherein the linker is

(SEQ ID NO: 130)
AAASGGSGGGGSGGGGSGP,
(SEQ ID NO: 131)
AAASGGSGGGGSGGGGS,
(SEQ ID NO: 132)
GGGGSGGGGSGGGGSGGGGS,
(SEQ ID NO: 36)
GGGGSGGGGSGGGGS,
(SEQ ID NO: 133)
GGGGSGGGGS,
(SEQ ID NO: 134)
GGGGS,
(SEQ ID NO: 135)
GGGGGGGG,
(SEQ ID NO: 136)
GGGGGG,
(SEQ ID NO: 137)
GSAGSAAGSGEF,
and
(SEQ ID NO: 138)
VPGVGVPGVG.

77. The recombinant protein system of aspect 74, wherein the linker is rigid.
78. The recombinant protein system of aspect 77, wherein the linker is

(SEQ ID NO: 139)
PAPAP,
(SEQ ID NO: 140)
EAAAKEAAAKEAAAK,
(SEQ ID NO: 141)
EAAAKEAAAK,
(SEQ ID NO: 142)
EAAAK,
(SEQ ID NO: 143)
AEAAAKEAAAKEAAAKEAAAKALEAEAAAKEAAAKEAAAKEAAAKA,
(SEQ ID NO: 144)
AEAAAKEAAAKA,
(SEQ ID NO: 145)
ESKYGPPCPPCP,
(SEQ ID NO: 146)
CPPCPAPELLGGPSVF,

and and alanine-proline (AP) repeated for a total of 10 to 34 amino acids (SEQ ID NO: 147).
79. The recombinant protein system of any one of aspects 69-78, wherein the targeting molecule is N terminal to the polypeptide catcher.
80. The recombinant protein system of any one of aspects 69-78, wherein the targeting molecule is C terminal to the polypeptide catcher.
81. The recombinant protein system of any one of aspects 45-80, wherein the second recombinant fusion protein comprises a signal peptide.
82. The recombinant protein system of any one of aspects 45-80, wherein the second recombinant fusion protein comprises a tag for western detection or purification.
83. The recombinant protein system of aspect 82, wherein the tag is a tag for western detection.
84. The recombinant protein system of aspect 82, wherein the tag is a tag for purification.
85. A membraned vesicle comprising, consisting essentially of, or consisting of the recombinant fusion protein of any one of aspects 1-44 or the recombinant protein system of any one of aspects 45-84.
86. The membraned vesicle of aspect 85, wherein the vesicle is a gesicle or an exosome.
87. The membraned vesicle of aspect 85 or 86, wherein the membraned vesicle comprises a mixed trimer, wherein the mixed trimer comprises (a) at least one recombinant fusion protein, wherein the polypeptide tag of the recombinant fusion protein is covalently bound to the polypeptide catcher of the second recombinant fusion protein, and (b) at least one (i) recombinant fusion protein, wherein the polypeptide tag of the recombinant fusion protein is not covalently bound to a polypeptide catcher or (ii) rhabdoviral G glycoprotein or functional fragment or derivative thereof that is not within a recombinant fusion protein.
88. The membraned vesicle of aspect 87, wherein the membraned vesicle comprises an unmixed trimer, wherein the unmixed trimer comprises only rhabdoviral G glycoprotein or a functional fragment or derivative thereof that is not within a recombinant fusion protein.
89. The membraned vesicle of aspect 88, wherein the membraned vesicle comprises the maximum number of the recombinant fusion proteins, wherein the polypeptide tag of the recombinant fusion protein is covalently bound to the polypeptide catcher of the second recombinant fusion protein, possible to be accommodated within the membrane of the membraned vesicle.
90. An enveloped viral particle comprising, consisting essentially of, or consisting of the recombinant fusion protein of any one of aspects 1-44 or the recombinant protein system of any one of aspects 45-84.
91. The enveloped viral particle of aspect 90, wherein the enveloped viral particle comprises a mixed trimer, wherein the mixed trimer comprises (a) at least one recombinant fusion protein, wherein the polypeptide tag of the recombinant fusion protein is covalently bound to the polypeptide catcher of the second recombinant fusion protein, and (b) at least one (i) recombinant fusion protein, wherein the polypeptide tag of the recombinant fusion protein is not covalently bound to a polypeptide catcher or (ii) rhabdoviral G glycoprotein or functional fragment or derivative thereof that is not within a recombinant fusion protein.
92. The enveloped viral particle of aspect 91, wherein the enveloped viral particle comprises an unmixed trimer, wherein the unmixed trimer comprises only rhabdoviral G glycoprotein or a functional fragment or derivative thereof that is not within a recombinant fusion protein.
93. The enveloped viral particle of aspect 92, wherein the enveloped viral particle comprises the maximum number of the recombinant fusion proteins possible to be accommodated within the membrane of the enveloped viral particle.
94. A recombinant viral vector comprising, consisting essentially of, or consisting of a nucleotide encapsulated by the membraned vesicle of any one of aspects 85-89 or the enveloped viral particle of any one of aspects 90-93.
95. A composition comprising, consisting essentially of, or consisting of a pharmaceutically acceptable carrier and the membraned vesicle of any one of aspects 85-89, the enveloped viral particle of any one of aspects 90-93, or the recombinant viral vector of aspect 94.
96. A method of delivering a payload to a target cell, the method comprising, consisting essentially of, or consisting of contacting the target cell with the membraned vesicle of any one of aspects 85-89, the enveloped viral particle of any one of aspects 90-93, the recombinant viral vector of aspect 94, or the composition of aspect 95.
97. The method of aspect 96, wherein the target cell in in vitro or ex vivo.
98. The method of aspect 96, wherein the target cell in in vivo.
99. The method of any one of aspects 96-98, wherein the payload is a nucleotide sequence encoding a chimeric antigen receptor (CAR) comprising, consisting essentially of, or consisting of an antigen binding domain, a transmembrane domain, and an intracellular signaling domain.
100. A retroviral vector expression system comprising, consisting essentially of, or consisting of one or more nucleotide sequences encoding the recombinant fusion protein of any one of aspects 1-44 or the recombinant protein system of any one of aspects 45-84.
101. The retroviral vector expression system of aspect 100, wherein the retroviral vector expression system comprises a vector construct and helper constructs that are each on separate plasmids.
102. The retroviral vector expression system of aspect 100 or 101, wherein the retroviral vector expression system is a lentirival vector expression system.
103. The retroviral vector expression system of any one of aspects 100-102, comprising, consisting essentially of, or consisting of a nucleotide sequence encoding a chimeric antigen receptor (CAR) comprising, consisting essentially of, or consisting of an antigen binding domain, a transmembrane domain, and an intracellular signaling domain.
104. A method of making a membraned vesicle, an enveloped viral particle, or a recombinant viral vector, the method comprising, consisting essentially of, or consisting of:
a. transfecting or transducing a packaging host cell with the retroviral vector expression system of any one of aspects 100-103; and
b. recovering the membraned vesicle, enveloped viral particle, or recombinant viral vector produced by the transfected or transduced packaging host cell.
105. A method of making a membraned vesicle, an enveloped viral particle, or a recombinant viral vector, the method comprising, consisting essentially of, or consisting of:
a. transfecting or transducing a first host cell with a first vector expression system comprising, consisting essentially of, or consisting of one or more nucleotide sequences encoding the recombinant fusion protein of any one of aspects 1-24, wherein the first host cell is a packaging host cell;
b. transfecting or transducing a second host cell with a second vector expression system comprising, consisting essentially of, or consisting of one or more nucleotide sequences encoding the second recombinant fusion protein;
c. collecting supernatant from the transfected or transduced second host cell;
d. administering the supernatant to the transfected or transduced first host cell; and
e. recovering the membraned vesicle, enveloped viral particle, or recombinant viral vector produced by the transfected or transduced first host cell after administration of supernatant produced by the transfected or transduced second host cell.
106. The method of aspect 105, wherein the polypeptide tag and the polypeptide catcher are the pair SpyTag and SpyCatcher (SEQ ID NO: 1 and SEQ ID NO: 30), SpyTag002 and SpyCatcher002 (SEQ ID NO: 2 and SEQ ID NO: 31), SpyTag003 and SpyCatcher003 (SEQ ID NO: 3 and SEQ ID NO: 32), SpyTag and KTag (SEQ ID NO: 1 and SEQ ID NO: 33), Isopeptag and pilin-C(SEQ ID NO: 4 and SEQ ID NO: 34), or SnoopTag and SnoopCatcher (SEQ ID NO: 5 and SEQ ID NO: 35).
107. The method of aspect 105 or 106, wherein the second recombinant fusion protein comprises a targeting molecule.
108. The method of aspect 107, wherein the targeting molecule is a ligand.
109. The method of aspect 108, wherein the ligand is epidermal growth factor (EGF), a mutant EGF (EFGm123) (SEQ ID NO: 59), stem cell factor (SCF) (SEQ ID NO: 52), thrombopoietin (TPO) (SEQ ID NO: 55), human hepatocyte growth factor (HGF) (SEQ ID NO: 56), or type 1 insulin-like growth factor (IGF1) (SEQ ID NO: 58).
110. The method of aspect 107, wherein the targeting molecule is a single chain variable fragment (scFv).
111. The method of aspect 110, wherein the scFv binds epidermal growth factor (EGF), human epidermal growth factor receptor 2 (Her2), cluster of differentiation 3 (CD3), or cluster of differentiation 117 (cKit), mucin-16 (MUC16), B cell maturation antigen (BCMA), or Nectin4.
112. The method of any one of aspects 107-111, wherein the second recombinant fusion protein comprises a linker between the targeting molecule and the polypeptide catcher.
113. The method of aspect 112, wherein the linker is flexible.
114. The method of aspect 113, wherein the linker is

(SEQ ID NO: 130)
AAASGGSGGGGSGGGGSGP,
(SEQ ID NO: 131)
AAASGGSGGGGSGGGGS,
(SEQ ID NO: 132)
GGGGSGGGGSGGGGSGGGGS,
(SEQ ID NO: 36)
GGGGSGGGGSGGGGS,
(SEQ ID NO: 133)
GGGGSGGGGS,
(SEQ ID NO: 134)
GGGGS,
(SEQ ID NO: 135)
GGGGGGGG,
(SEQ ID NO: 136)
GGGGGG,
(SEQ ID NO: 137)
GSAGSAAGSGEF,
and
(SEQ ID NO: 138)
VPGVGVPGVG.

115. The method of aspect 112, wherein the linker is rigid.
116. The method of aspect 115, wherein the linker is

(SEQ ID NO: 139)
PAPAP,
(SEQ ID NO: 140)
EAAAKEAAAKEAAAK,
(SEQ ID NO: 141)
EAAAKEAAAK,
(SEQ ID NO: 142)
EAAAK,
(SEQ ID NO: 143)
AEAAAKEAAAKEAAAKEAAAKALEAEAAAKEAAAKEAAAKEAAAKA,
(SEQ ID NO: 144)
AEAAAKEAAAKA,
(SEQ ID NO: 145)
ESKYGPPCPPCP,
(SEQ ID NO: 146)
CPPCPAPELLGGPSVF,

and and alanine-proline (AP) repeated for a total of 10 to 34 amino acids (SEQ ID NO: 147).
117. The method of any one of aspects 107-116, wherein the targeting molecule is N terminal to the polypeptide catcher.
118. The method of any one of aspects 107-116, wherein the targeting molecule is C terminal to the polypeptide catcher.
119. The method of any one of aspects 105-118, wherein the second recombinant fusion protein comprises a signal peptide.
120. The method of any one of aspects 105-119, wherein the second recombinant fusion protein comprises a tag for western detection or purification.
121. The method of aspect 120, wherein the tag is a tag for western detection.
122. The method of aspect 120, wherein the tag is a tag for purification.
123. The method of aspect any one of aspects 105-122, wherein the packaging host cell is HEK 293T or a 293 cell suspension line.
124. A plasmid comprising, consisting essentially of, or consisting of one or more nucleotide sequences encoding the recombinant fusion protein of any one of aspects 1-44 or the recombinant protein system of any one of aspects 45-84.
125. The composition of aspect 95 or the retroviral vector expression system of any one of aspects 100-103 for use in treating a disease in a mammal.
126. The method of aspect 125, wherein the mammal is a human.
127. The method of aspect 125 or 126, wherein the disease is a genetic disease.
128. The method of aspect 125 or 126, wherein the disease is a cancer.
129. A method of making a mixed rhabdoviral G glycoprotein trimer, the method comprising, consisting essentially of, or consisting of:
a. transfecting or transducing a packaging host cell with the retroviral vector expression system of any one of aspects 100-103; and
b. recovering the mixed rhabdoviral G glycoprotein trimer.
130. A method of making a mixed rhabdoviral G glycoprotein trimer, the method comprising, consisting essentially of, or consisting of:
a. transfecting or transducing a first host cell with a first vector expression system comprising, consisting essentially of, or consisting of one or more nucleotide sequences encoding the recombinant fusion protein of any one of aspects 1-24, wherein the first host cell is a packaging host cell;
b. transfecting or transducing a second host cell with a second vector expression system comprising, consisting essentially of, or consisting of one or more nucleotide sequences encoding the second recombinant fusion protein;
c. contacting virus produced by the first host cell with second recombinant fusion protein produced by the second host cell; and
d. obtaining virus decorated with the second recombinant fusion protein,
   wherein the virus comprises a mixed trimer, wherein the mixed trimer comprises (a) at least one recombinant fusion protein, wherein the polypeptide tag of the recombinant fusion protein is covalently bound to the polypeptide catcher of the second recombinant fusion protein, and (b) at least one (i) recombinant fusion protein, wherein the polypeptide tag of the recombinant fusion protein is not covalently bound to a polypeptide catcher or (ii) rhabdoviral G glycoprotein or a functional fragment or derivative thereof that is not within a recombinant fusion protein.
131. The method of aspect 130, wherein the polypeptide tag and the polypeptide catcher are the pair SpyTag and SpyCatcher (SEQ ID NO: 1 and SEQ ID NO: 30), SpyTag002 and SpyCatcher002 (SEQ ID NO: 2 and SEQ ID NO: 31), SpyTag003 and SpyCatcher003 (SEQ ID NO: 3 and SEQ ID NO: 32), SpyTag and KTag (SEQ ID NO: 1 and SEQ ID NO: 33), Isopeptag and pilin-C(SEQ ID NO: 4 and SEQ ID NO: 34), or SnoopTag and SnoopCatcher (SEQ ID NO: 5 and SEQ ID NO: 35).
132. The method of aspect 130 or 131, wherein the second recombinant fusion protein comprises a targeting molecule.
133. The method of aspect 132, wherein the targeting molecule is a ligand.
134. The method of aspect 133, wherein the ligand is epidermal growth factor (EGF), a mutant EGF (EFGm123) (SEQ ID NO: 59), stem cell factor (SCF) (SEQ ID NO: 52), thrombopoietin (TPO) (SEQ ID NO: 55), human hepatocyte growth factor (HGF) (SEQ ID NO: 56), or type 1 insulin-like growth factor (IGF1) (SEQ ID NO: 58).
135. The method of aspect 132, wherein the targeting molecule is a single chain variable fragment (scFv).
136. The method of aspect 135, wherein the scFv binds epidermal growth factor (EGF), human epidermal growth factor receptor 2 (Her2), cluster of differentiation 3 (CD3), or cluster of differentiation 117 (cKit), mucin-16 (MUC16), B cell maturation antigen (BCMA), or Nectin4.
137. The method of any one of aspects 132-136, wherein the second recombinant fusion protein comprises a linker between the targeting molecule and the polypeptide catcher.
138. The method of aspect 137, wherein the linker is flexible.
139. The method of aspect 138, wherein the linker is

(SEQ ID NO: 130)
AAASGGSGGGGSGGGGSGP,
(SEQ ID NO: 131)
AAASGGSGGGGSGGGGS,
(SEQ ID NO: 132)
GGGGSGGGGSGGGGSGGGGS,
(SEQ ID NO: 36)
GGGGSGGGGSGGGGS,
(SEQ ID NO: 133)
GGGGSGGGGS,
(SEQ ID NO: 134)
GGGGS,
(SEQ ID NO: 135)
GGGGGGGG,
(SEQ ID NO: 136)
GGGGGG,
(SEQ ID NO: 137)
GSAGSAAGSGEF,
and
(SEQ ID NO: 138)
VPGVGVPGVG.

140. The method of aspect 137, wherein the linker is rigid.
141. The method of aspect 140, wherein the linker is

(SEQ ID NO: 139)
PAPAP,
(SEQ ID NO: 140)
EAAAKEAAAKEAAAK,
(SEQ ID NO: 141)
EAAAKEAAAK,
(SEQ ID NO: 142)
EAAAK,
(SEQ ID NO: 143)
AEAAAKEAAAKEAAAKEAAAKALEAEAAAKEAAAKEAAAKEAAAKA,
(SEQ ID NO: 144)
AEAAAKEAAAKA,
(SEQ ID NO: 145)
ESKYGPPCPPCP,
(SEQ ID NO: 146)
CPPCPAPELLGGPSVF,

and
   alanine-proline (AP) repeated for a total of 10 to 34 amino acids (SEQ ID NO: 147).
142. The method of any one of aspects 132-141, wherein the targeting molecule is N terminal to the polypeptide catcher.
143. The method of any one of aspects 132-141, wherein the targeting molecule is C terminal to the polypeptide catcher.
144. The method of any one of aspects 130-143, wherein the second recombinant fusion protein comprises a signal peptide.
145. The method of any one of aspects 130-144, wherein the second recombinant fusion protein comprises a tag for western detection or purification.
146. The method of aspect 145, wherein the tag is a tag for western detection.
147. The method of aspect 145, wherein the tag is a tag for purification.
148. The method of aspect any one of aspects 130-147, wherein the packaging host cell is HEK 293T or a 293 cell suspension line.
149. A method of reducing inhibition of rhabdoviral G glycoprotein-mediated fusion in serum, wherein the inhibition is by complement inactivation or competitive binding with LDL or vLDL, the method comprising, consisting essentially of, or consisting of covalently bonding to a rhabdoviral G glycoprotein or functional fragment or derivative thereof a targeting molecule, and exposing the rhabdoviral G glycoprotein or functional fragment or derivative thereof with targeting molecule to serum, wherein complement inactivation or competitive binding with LDL or vLDL is inhibited.
150. The method of aspect 149, wherein the targeting molecule is a ligand.
151. The method of aspect 150, wherein the ligand is epidermal growth factor (EGF), a mutant EGF (EFGm123) (SEQ ID NO: 59), stem cell factor (SCF) (SEQ ID NO: 52), thrombopoietin (TPO) (SEQ ID NO: 55), human hepatocyte growth factor (HGF) (SEQ ID NO: 56), or type 1 insulin-like growth factor (IGF1) (SEQ ID NO: 58).
152. The method of aspect 149, wherein the targeting molecule is a single chain variable fragment (scFv).
153. The method of aspect 152, wherein the scFv binds epidermal growth factor (EGF), human epidermal growth factor receptor 2 (Her2), cluster of differentiation 3 (CD3), or cluster of differentiation 117 (cKit), mucin-16 (MUC16), B cell maturation antigen (BCMA), or Nectin4.
154. The method of any one of aspects 149-153, wherein the second recombinant fusion protein comprises a linker between the targeting molecule and the polypeptide catcher.
155. The method of aspect 154, wherein the linker is flexible.
156. The method of aspect 155, wherein the linker is

(SEQ ID NO: 130)
AAASGGSGGGGSGGGGSGP,
(SEQ ID NO: 131)
AAASGGSGGGGSGGGGS,
(SEQ ID NO: 132)
GGGGSGGGGSGGGGSGGGGS,
(SEQ ID NO: 36)
GGGGSGGGGSGGGGS,
(SEQ ID NO: 133)
GGGGSGGGGS,
(SEQ ID NO: 134)
GGGGS,
(SEQ ID NO: 135)
GGGGGGGG,
(SEQ ID NO: 136)
GGGGGG,
(SEQ ID NO: 137)
GSAGSAAGSGEF,
and
(SEQ ID NO: 138)
VPGVGVPGVG.

157. The method of aspect 154, wherein the linker is rigid.
158. The method of aspect 157, wherein the linker is

(SEQ ID NO: 139)
PAPAP,
(SEQ ID NO: 140)
EAAAKEAAAKEAAAK,
(SEQ ID NO: 141)
EAAAKEAAAK,
(SEQ ID NO: 142)
EAAAK,
(SEQ ID NO: 143)
AEAAAKEAAAKEAAAKEAAAKALEAEAAAKEAAAKEAAAKEAAAKA,
(SEQ ID NO: 144)
AEAAAKEAAAKA,
(SEQ ID NO: 145)
ESKYGPPCPPCP,
(SEQ ID NO: 146)
CPPCPAPELLGGPSVF,

and
   alanine-proline (AP) repeated for a total of 10 to 34 amino acids (SEQ ID NO: 147).
159. A method of reducing inhibition of rhabdoviral G glycoprotein-mediated fusion in serum, wherein the inhibition is by complement inactivation or competitive binding with LDL or vLDL, the method comprising, consisting essentially of, or consisting of producing the rhabdoviral G glycoprotein or functional fragment or derivative thereof as the rhabdoviral G glycoprotein or functional fragment or derivative thereof within the recombinant fusion protein of any one of aspects 25-44, and exposing the rhabdoviral G glycoprotein or functional fragment or derivative thereof to serum, wherein complement inactivation or competitive binding with LDL or vLDL is inhibited.
160. A method of genetically engineering a multimeric protein without significantly disrupting a natural or desired function of the multimeric protein, wherein the multimeric protein comprises identical or similar subunits, the method comprising: (a) genetically engineering a subunit of the multimeric protein to be within a recombinant fusion protein comprising (1) the subunit and (2) a protein ligand or targeting molecule; (b) expressing in a cell the recombinant fusion protein of (a); and (c) limiting the number of subunits expressed in (b) to provide multimeric proteins comprising less than full occupancy of subunits comprising the protein ligand or targeting molecule, wherein the genetically engineered multimeric protein exhibits a natural or a desired function that is not significantly disrupted compared to a wild-type multimeric protein.
161. The method of aspect 160, wherein the limiting of (c) is by co-expression of subunits using plasmids encoding (A) the recombinant fusion protein of (a) and (B) a subunit not comprising the protein ligand or targeting moiety.
162. The method of aspect 160, wherein the limiting of (c) is by use of a system comprising a polypeptide (polypeptide tag) that is capable of forming a covalent bond with an amino acid residue in a second recombinant fusion protein, wherein the polypeptide tag is N-terminal to the rhabdoviral G glycoprotein, and wherein the second recombinant fusion protein comprises a polypeptide (polypeptide catcher) that is capable of forming a covalent bond with an amino acid residue in the polypeptide tag.

It shall be noted that the preceding are merely examples of aspects of the disclosure. Other exemplary aspects are apparent from the entirety of the description herein. It will also be understood by one of ordinary skill in the art that each of these aspects may be used in various combinations with the other aspects provided herein.

The following examples further illustrate aspects of the disclosure, but, of course, should not be construed as in any way limiting its scope.

### Example 1

This example demonstrates the development of constructs for recombinant rhabdoviral G glycoproteins with SpyTag insertions.

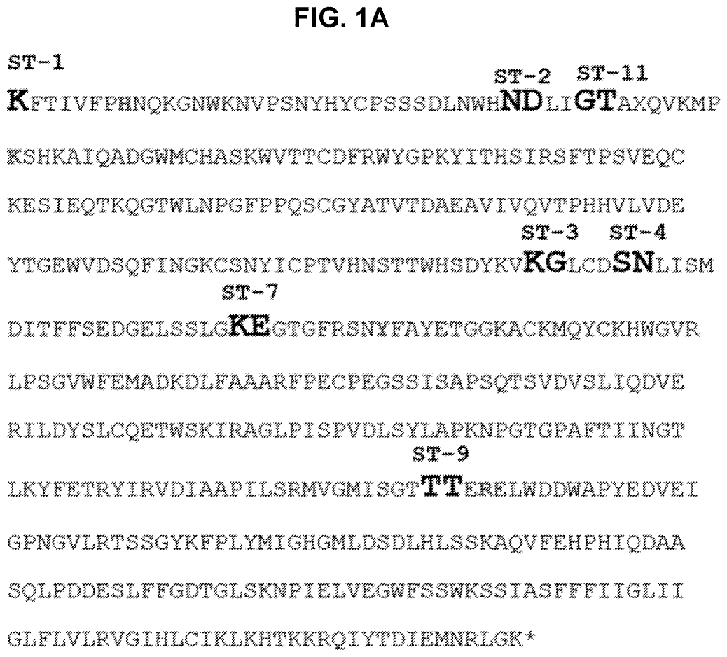

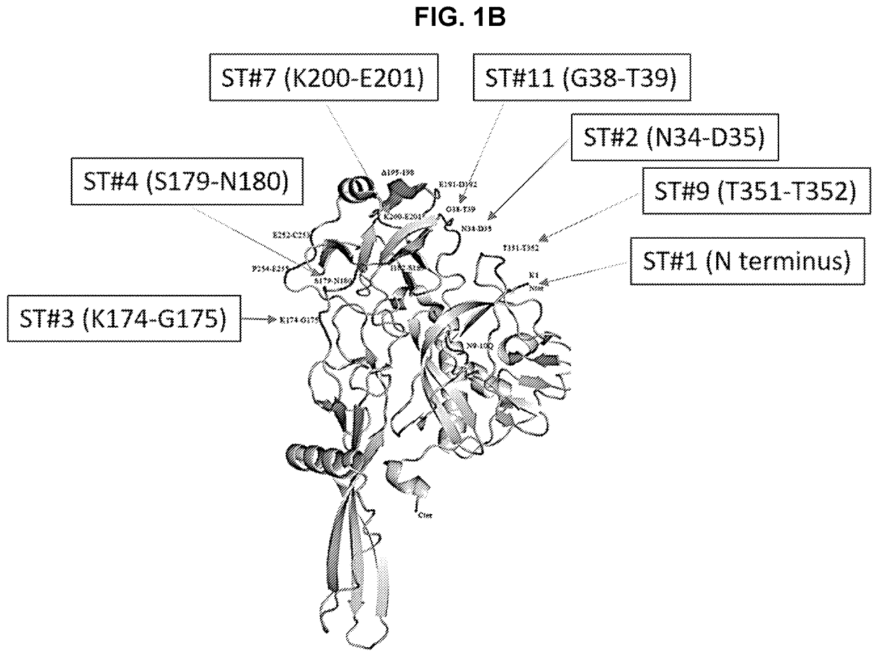

To develop a rhabdoviral G glycoprotein recombinant fusion protein that could covalently bond to a second recombinant fusion protein comprising SpyCatcher, SpyTag insertion sites were identified in the Vesiculovirus indiana glycoprotein (VSIV-G) (SEQ ID NO: 21) (FIG. 1A). SpyTag insertion sites were also characterized in the context of the crystal structure of VSIV-G (SEQ ID NO: 77) (FIG. 1B). The PyMOL™ program was used to edit the G glycoprotein crystal structure. Various VSIV-G constructs were developed with the identified SpyTag insertion sites as shown in Table 2. These constructs were developed with a flexible linker (GSGGSG) (SEQ ID NO: 12) flanking the SpyTag insertion site, except at insertion site-1, where the linker is only between the SpyTag (SEQ ID NO: 1) 3′ terminus and the VSIV-G 5′ terminus (SEQ ID NO: 21).

TABLE 2

| | | SEQ | SpyTag |
|---|---|---|---|
| Site | | ID | Insertion |
| No. | Name of constructs | NO | Location |
| 1 | VSV-G-WT-ST-1 | 78 | ST-K1 |
| 2 | VSV-G-WT-ST-2 | 79 | N34-ST-D35 |
| 3 | VSV-G-WT-ST-3 | 80 | K174-ST-G175 |
| 4 | VSV-G-WT-ST-4 | 81 | S179-ST-N180 |
| 7 | VSV-G-WT-ST-7 | 82 | K200-ST-E201 |
| 9 | VSV-G-WT-ST-9 | 83 | T351-ST-T352 |
| 11 | VSV-G-WT-ST-11 | 84 | G38-ST-T39 |

### Example 2

This example demonstrates how linkers affect SpyTag construct expression, localization, and function.

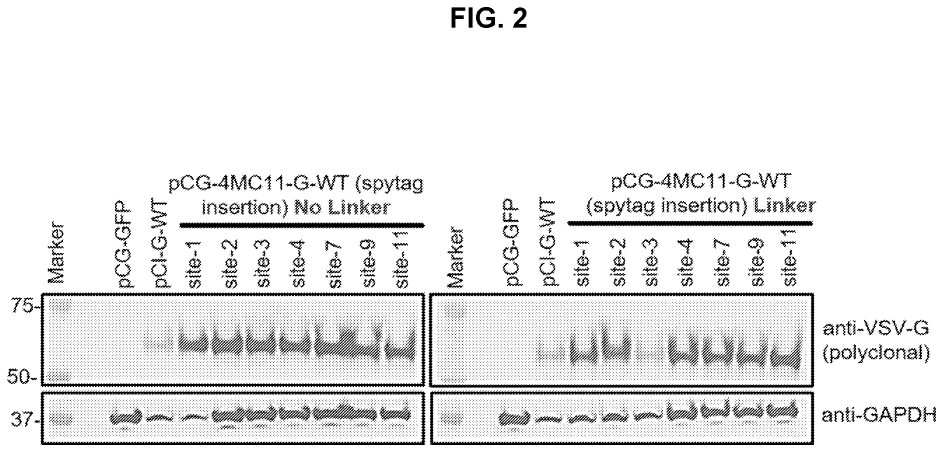

To test that the SpyTag VSIV-G constructs are efficiently translated, they were placed into pCG expression vectors both with and without linkers, and then these plasmids were transfected in HEK-293T cells. pCG expression vectors with GFP and pCI expression vectors with VSIV-G without SpyTag insertions were transfected into HEK-293T cells as controls. 48 hours after transfection cells were collected, lysed, and western blotting was run on the cell lysate using anti-VSV-G polyclonal and GAPDH antibodies (FIG. 2). Similar expression levels of VSIV-G was found for all the constructs suggesting the SpyTag insertion sites do not affect protein expression.

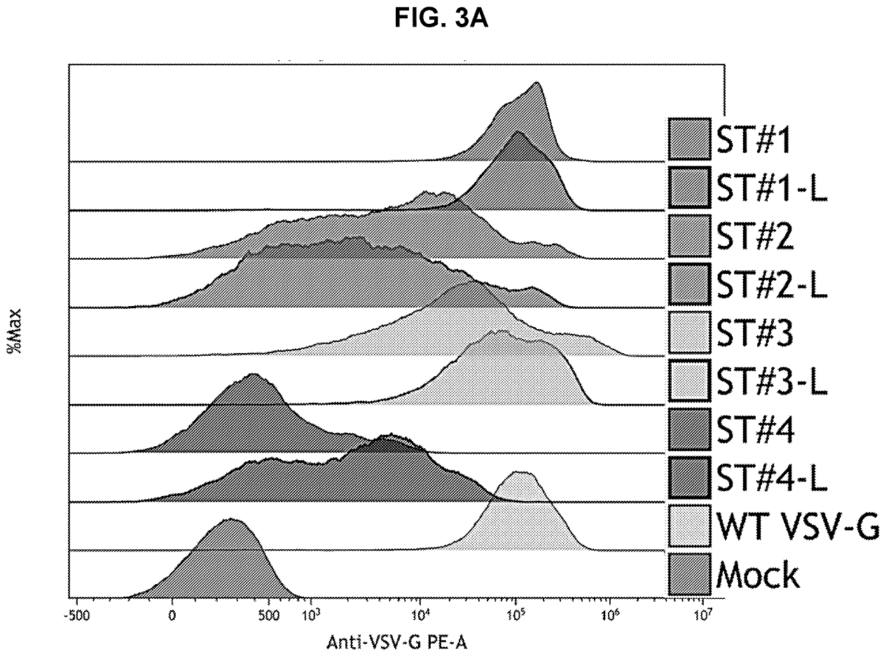

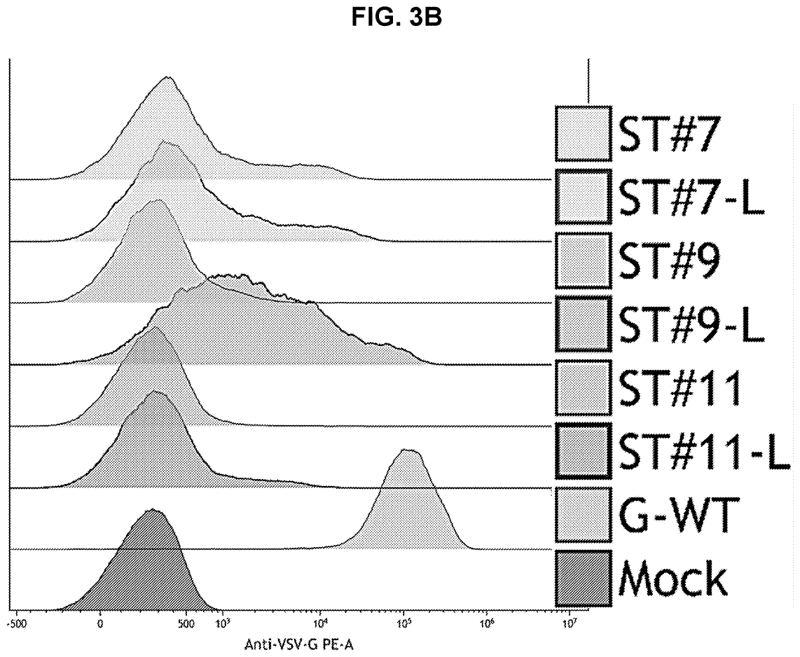

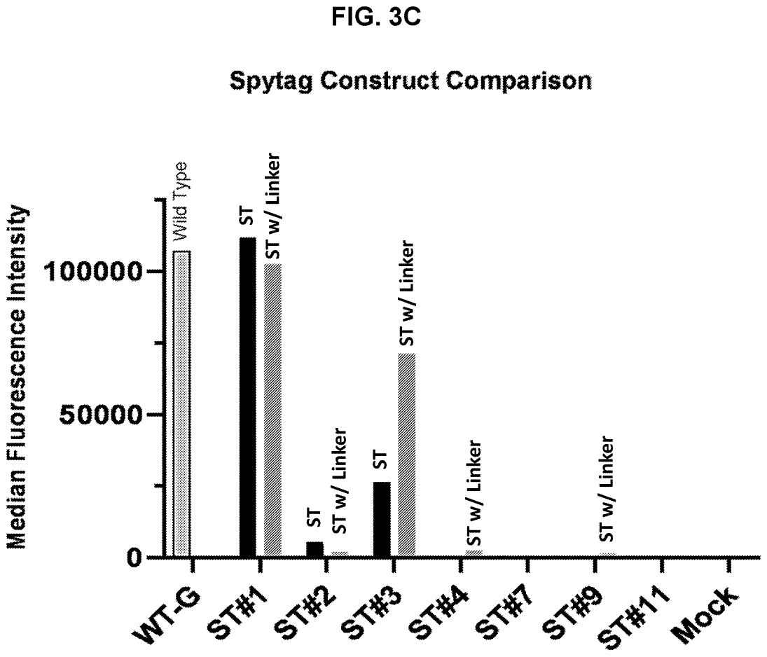

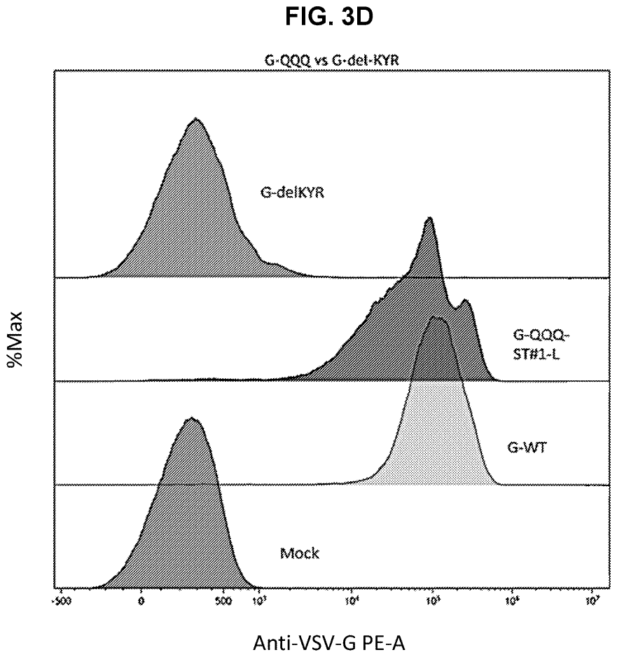

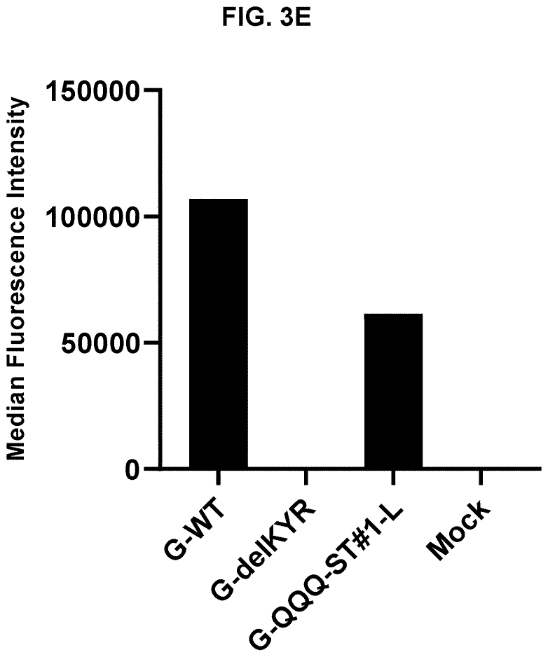

The ability of SpyTag VSIV-G recombinant fusion proteins to localize to the cell surface was tested using plasmids encoding SpyTag VSIV-G recombinant fusion proteins with or without linkers. HEK293T cells were transfected with the plasmids encoding SpyTag VSIV-G without (ST #1, ST #2, ST #3, ST #4, ST #7, ST #9, and ST #11) or with linkers (ST #1-L, ST #2-L, ST #3-L, ST #4-L, ST #7-L, ST #9-L, and ST #11-L) as shown in Tables 2 and 3 respectively. Plasmids encoding wild type VSIV-G without SpyTag insertions (SEQ ID NO: 96) and Mock constructs were used as controls. 24 hours after transfection a fluorescently labeled anti-VSIV G antibody was used to stain VSIV-G glycoproteins on the cell surface. G glycoprotein expression was detected by flow cytometry (FIGS. 3A-3B). The cell surface localization of VSIV-G constructs with mutations to reduce or abolish binding to LDL receptors was also tested in the same manner using plasmids encoding VSIV-G with a K47+R354+Y209 deletion (G-delKYR) (SEQ ID NO: 28) or a SpyTag K47Q+R354Q+Y209Q substitution VSIV-G (G-QQQ-ST #1-L) (SEQ ID NOs: 66 and 93) (FIG. 3D). The insertion of SpyTag at site-1 is well tolerated and had no effect on cell surface expression, showing similar levels to wild type VSIV-G (FIG. 3C). Insertion of SpyTag was also tolerated to a lesser extent at site-2 and site-3. However, insertion sites 4, 7, 9, and 11 did not tolerate the SpyTag insertion well. The insertion of SpyTag at site-1 is also well tolerated in the K47Q+R354Q+Y209Q substitution VSIV-G (SEQ ID NOs: 66 and 93) (FIG. 3E) but not well tolerated in the SpyTag K47+R354+Y209 deletion (G-delKYR) constructs (SEQ ID NO: 92). This suggests that SpyTag insertion is only tolerated at particular sites in the VSIV-G. The linker (GSGGSG) (SEQ ID NO: 12) flanking SpyTag in the VSIV-G does provide some advantage on the cell surface localization of VSIV-G for some of the G-proteins among the tested constructs, particularly insertion site-3 (FIG. 3C). Not wishing to be bound by theory, this suggests the linker may provide some advantage, possibly by adding some flexibility to the protein trafficking and cell surface localization. The VSIV-G with K47+R354+Y209 deletion (G-delKYR) (SEQ ID NO: 28) failed to localize to the cell surface even without a SpyTag insertion. This suggests that deletion of K47+R354+Y209 VSIV-G residues can affect the protein folding and trafficking to the cell surface.

TABLE 3

| | | SEQ | SpyTag |
|---|---|---|---|
| Site | | ID | Insertion |
| No. | Name of constructs | NO | Location |
| 1 | VSV-G-WT-ST-L#1 | 85 | ST-K1 |
| 1 | VSV-G-(del K47Y209R354)- | 92 | ST-K1 |
| | ST-L#1 | | |
| 1 | VSV-G-(K47QY209QR354Q)- | 93 | ST-K1 |
| | ST-L#1 | | |
| 2 | VSV-G-WT-ST-L#2 | 86 | N34-ST-D35 |
| 3 | VSV-G-WT-ST-L#3 | 87 | K174-ST- |
| | | | G175 |
| 4 | VSV-G-WT-ST-L#4 | 88 | S179-ST- |
| | | | N180 |
| 7 | VSV-G-WT-ST-L#7 | 89 | K200-ST- |
| | | | E201 |
| 9 | VSV-G-WT-ST-L#9 | 90 | T351-ST- |
| | | | T352 |
| 11 | VSV-G-WT-ST-L#11 | 91 | G38-ST-T39 |

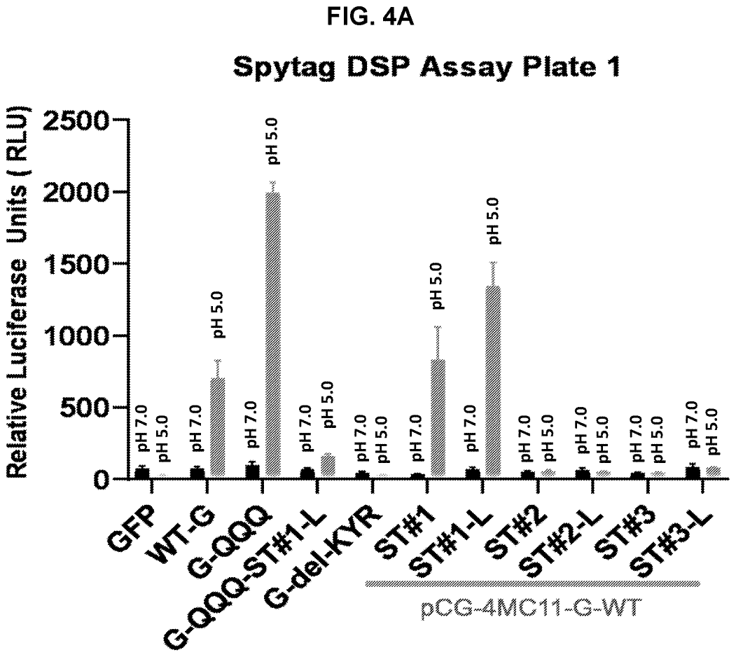

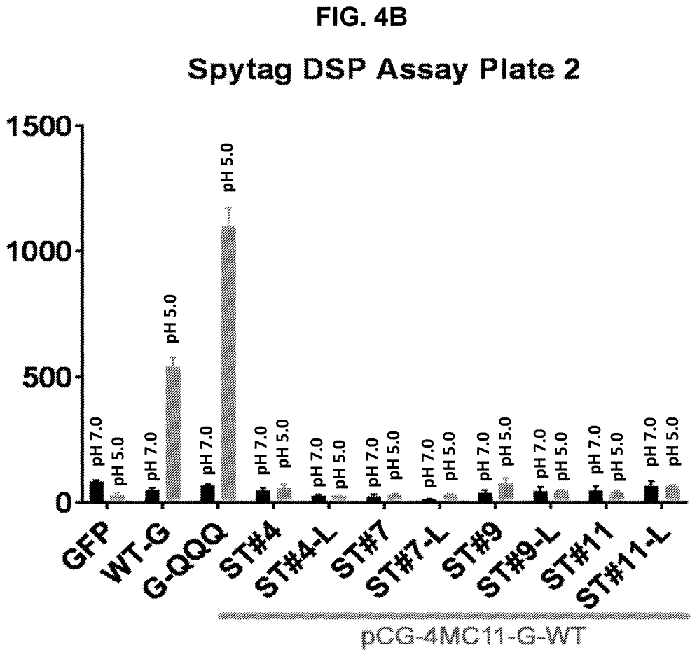

To determine if the SpyTag VSIV-G constructs with or without linkers were capable of inducing membrane fusion a dual split protein (DSP) assay was performed. Two distinct stable cell lines, expressing DSP-1 and DSP-2 fragments separately in K562, were co-cultured before plasmid transfection. After 24 hours, K562 cells were transduced with 10 ul of plasmids encoding SpyTag VSIV-G constructs with or without linkers as shown in Tables 2 and 3, VSIV-G with a K47+R354+Y209 deletion (G-del-KYR) (SEQ ID NO: 28), a K47Q+R354Q+Y209Q substitution VSIV-G (G-QQQ) (SEQ ID NO: 26), or a SpyTag K47Q+R354Q+Y209Q substitution VSIV-G with linker (G-QQQ-ST #1-L) (SEQ ID NO: 66). Plasmids encoding VSIV-G without a SpyTag insertion (WT-G) (SEQ ID NO: 21) were used as a positive control and plasmids with only GFP were used as a negative control (GFP). 24 hours after transfection, the cells were treated with pH 5.0 phosphate buffered saline (PBS) to induce fusion or pH 7.0 PBS as a control for 2 minutes, and then fresh media was added containing the luciferase substrate, EnduRen. Renilla luciferase activity was measured 4 hours after the addition of the substrate. The cells transfected with VSIV-G with K47Q+R354Q+Y209Q substitution (SEQ ID NO: 26), VSIV with SpyTag inserted at site-1 with (SEQ ID NO: 85) and without (SEQ ID NO: 78) a linker all showed relative luminescence levels comparable or higher than the wild type VSIV-G (FIGS. 4A and 4B). These results suggested that insertion of SpyTag at site-1, on the N-terminus, in the VSIV-G seems to be well tolerated and does not negatively impact G protein mediated membrane fusion.

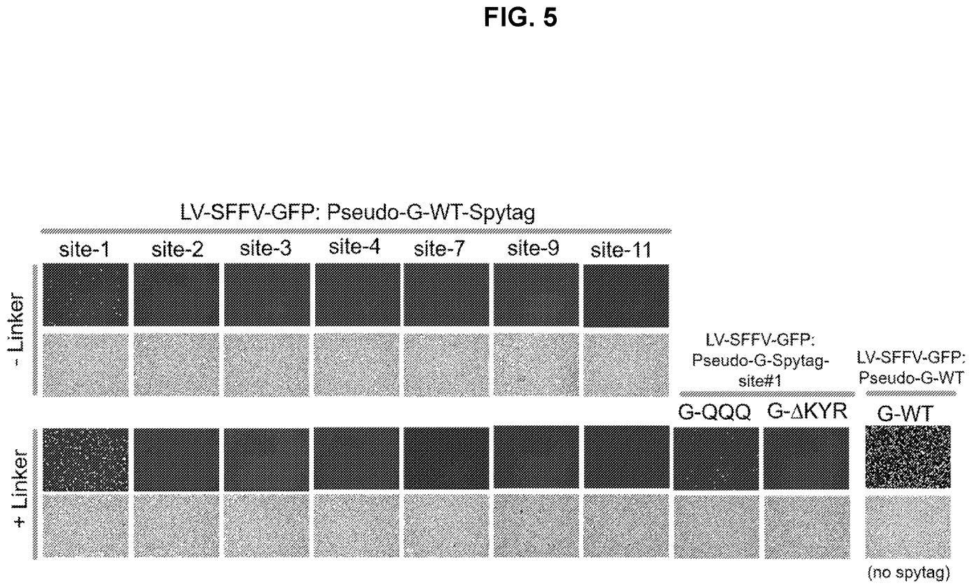

The effect of various SpyTag insertion sites in constructs with and without linkers on lentivirus infectivity was tested. K562 cells were transduced with 10 ul of lentiviruses containing a GFP expressing cassette pseudotyped with either VSIV-G-SpyTag with or without linker as shown in Tables 1 and 2, SpyTag VSIV-G with K47Q+R354Q+Y209Q substitution (G-QQQ) and linker (SEQ ID NO: 93), SpyTag VSIV-G with a K47+R354+Y209 deletion (G-AKYR) and linker (SEQ ID NO: 92), or VSIV-G without SpyTag (G-WT) (SEQ ID NO: 96). 72 hours after transduction, fluorescent (top) and phase contrast (bottom) micrographs were captured by Nikon microscope at 10× objective lens (FIG. 5). The wild type VSIV-G lentivirus infected cells showed GFP positive cells, but only the SpyTag VSIV-G lentiviruses pseudotyped with ST-1-VSV-G-WT (SEQ ID NO: 78), ST-1-L-VSV-G-WT (SEQ ID NO: 85), or ST-1-L-VSV-G-QQQ (SEQ ID NO: 93) resulted in GFP positive cells. These results further support that insertion of SpyTag at site-1, on the N-terminus of the VSIV-G is well tolerated. This also suggests that VSIV-G with SpyTag insertion at site-1 are capable of fusion and can support lentiviral infection.

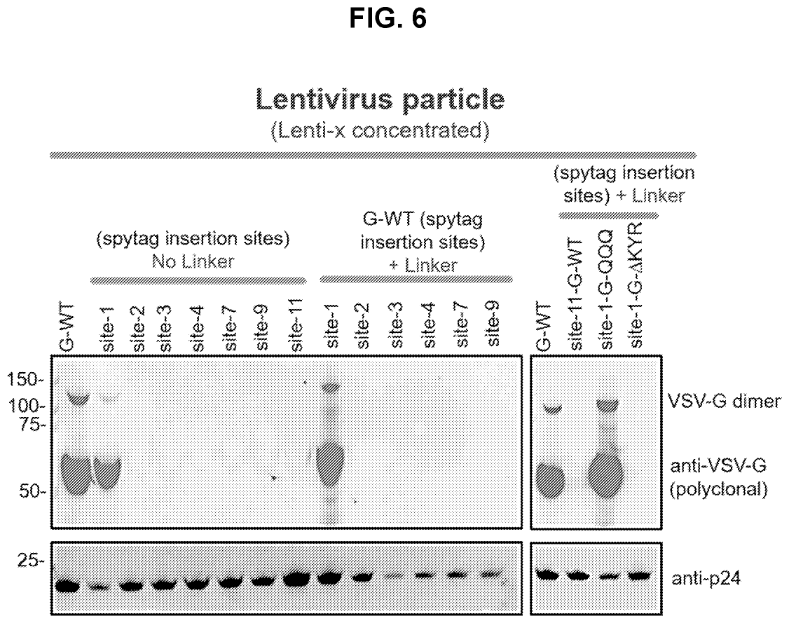

To further test the effects of SpyTag insertion with or without linkers in lentivirus western blotting analysis was performed. Lentiviruses were pseudotyped with VSIV-Gs with SpyTag inserted with or without linkers as shown in Tables 1 and 2, VSIV-G with K47Q+R354Q+Y209Q substitution (G-QQQ) with SpyTag and linker (SEQ ID NO: 93), VSIV-G with a K47+R354+Y209 deletion (G-AKYR) with SpyTag and linker (SEQ ID NO: 92), or VSIV-G without SpyTag (G-WT) (SEQ ID NO: 96). The lentiviruses were Lenti-X concentrated and 12.5 pd was loaded into each well. The samples were run on a 4-12% Bis-Tris Gel followed by western blotting with anti-VSIV-G polyclonal antibody and p24 antibody (FIG. 6). The western blotting of the virion pellet showed that the wild type VSIV-G lentivirus infected cells showed VSIV-G antibody binding, but only the lentiviruses pseudotyped with VSIV-G or VSIV-G-QQQ with SpyTag inserted at site 1 with and without a linker resulted in cells with VSIV-G antibody binding. These results further support that SpyTag insertion at site-1 is well tolerated for incorporation into the lentivirus particle.

### Example 3

This example demonstrates transfection using SpyTag VSIV-G encoding plasmids and SpyCatcher targeting molecule encoding plasmids.

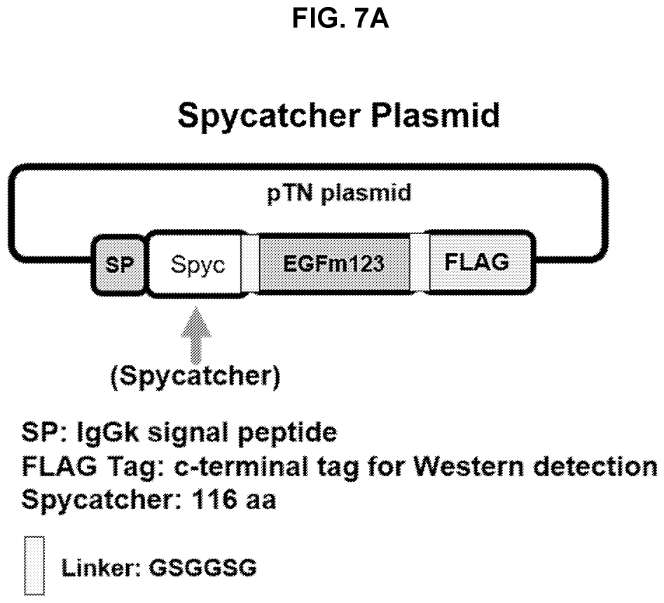

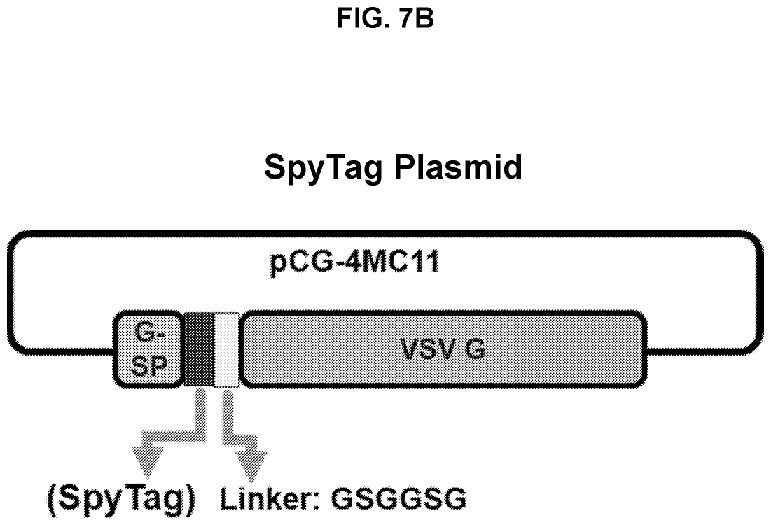

A targeting molecule plasmid, encoding SpyCatcher (the SpyCatcher meant for binding a VSIV-G with a SpyTag) was developed. The SpyCatcher targeting plasmid (SEQ ID NO: 99) was designed in a pTN expression plasmid to encode an immunoglobin kappa chain (IgGk) secretory signal peptide (SP) (SEQ ID NO: 61), SpyCatcher (SEQ ID NO: 30), linkers (SEQ ID NO: 36) flanking the targeting molecule, and a FLAG tag (SEQ ID NO: 62) at the C-terminus for western detection (FIG. 7A). The targeting molecule selected for this construct was a modified version of epidermal growth factor (EGFm123) (SEQ ID NO: 59). The SpyTag VSIV-G plasmid (SEQ ID NO: 93) used in these experiments was designed in a pCG-4MC11 expression plasmid to encode a G glycoprotein signal peptide (G-SP) (SEQ ID NO: 60), SpyTag (SEQ ID NO: 1) at insertion site-1, a linker (SEQ ID NO: 12), and VSIV-G with a K47Q+R354Q+Y209Q substitution (VSV G) (SEQ ID NO: 21) (FIG. 7B).

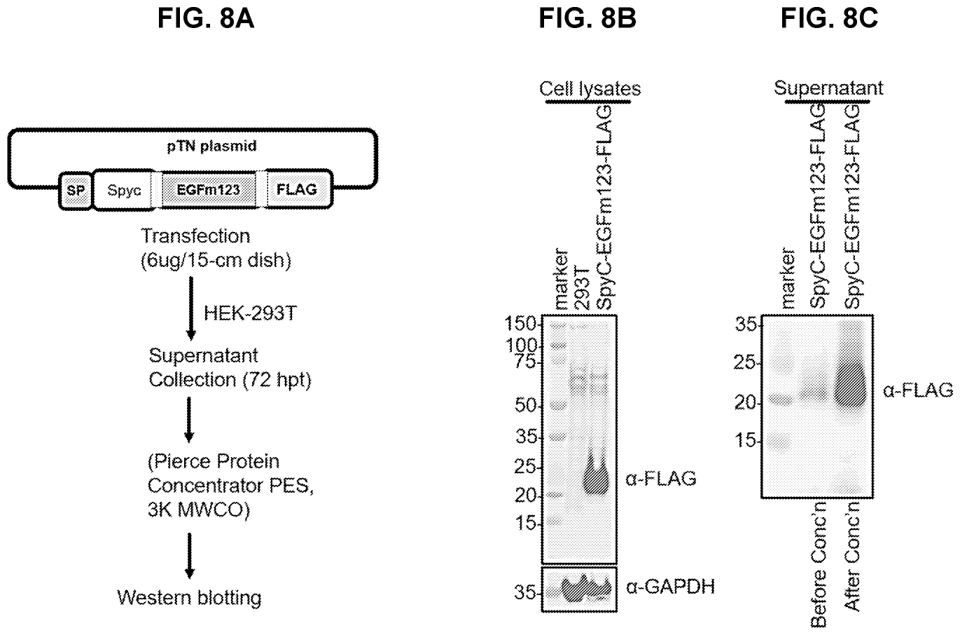

To test the transfection of the SpyCatcher targeting plasmid an experiment was performed as shown in FIG. 8A. HEK293T cells were transfected at a concentration of 6 μg per 15 cm dish. 72 hours after transfection supernatant was collected, clarified, passed through a 0.45 micron filter and then subjected to Pierce protein concentrator PES, 3K MWCO. Western blotting was performed on the cell lysates using anti-FLAG and anti-GAPDH antibodies (FIG. 8B). Western blotting was also performed on the supernatants collected before and after concentration using anti-FLAG antibodies (FIG. 8C). FLAG antibody binding was observed in both unconcentrated and concentrated supernatant suggesting that HEK293 cells secrete the SpyCatcher targeting molecule recombinant fusion protein. The darker band of FLAG antibody binding suggests that a SpyCatcher targeting molecule can be secreted and concentrated from the transfected HEK293T cells.

### Example 4

This example demonstrates the construction of targeted pseudotyped lentivirus particles by cotransfection or coexpression of the SpyCatcher plasmid with the lentiviral, envelope, and packaging plasmids.

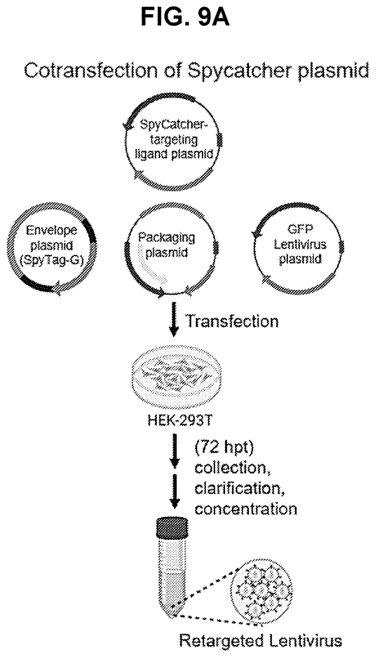

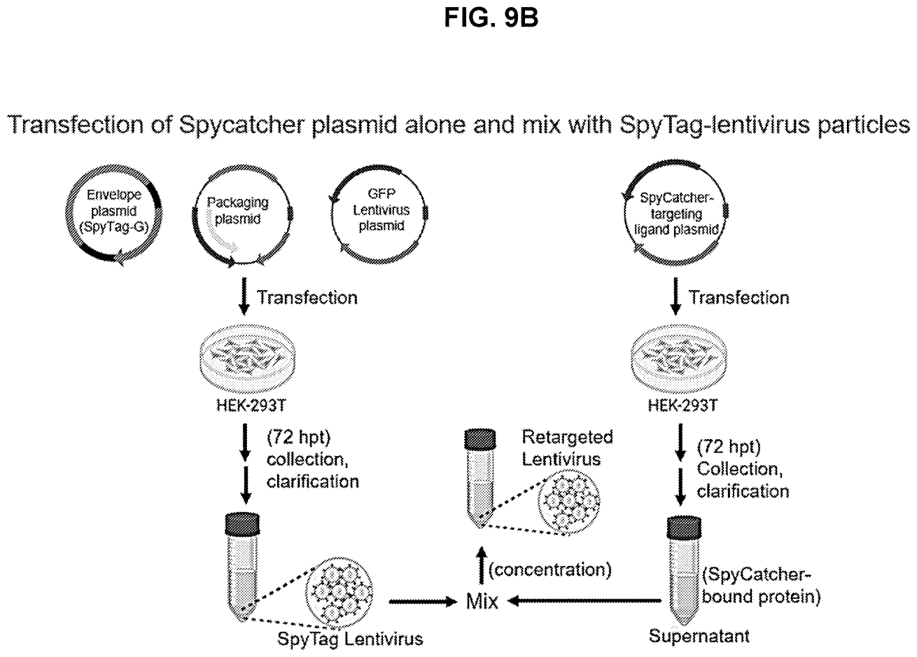

A method of cotransfecting the SpyCatcher targeting molecule recombinant fusion protein together with the VSIV-G SpyTag envelope plasmid, lentiviral plasmid, and packaging plasmid was developed as shown in FIG. 9A.

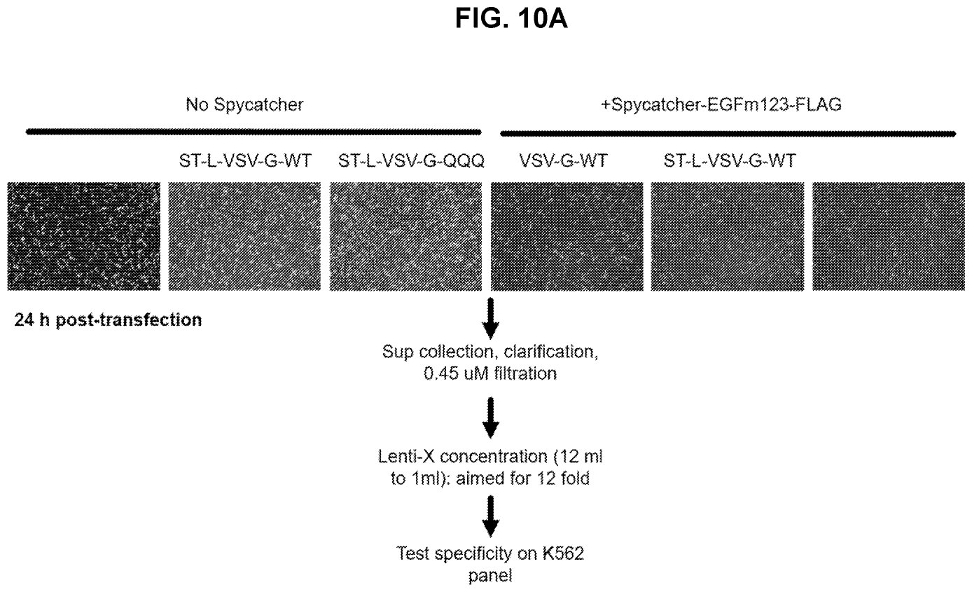

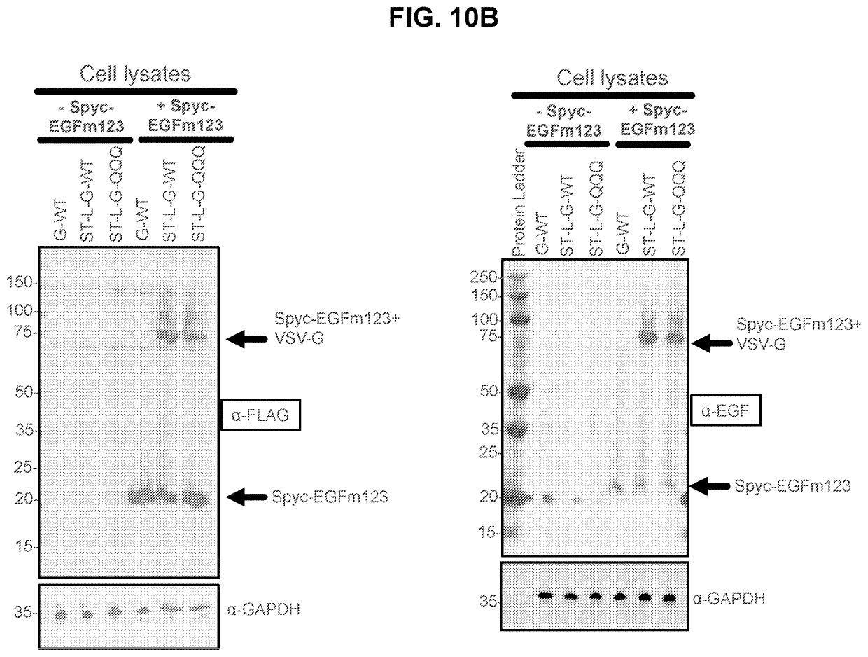

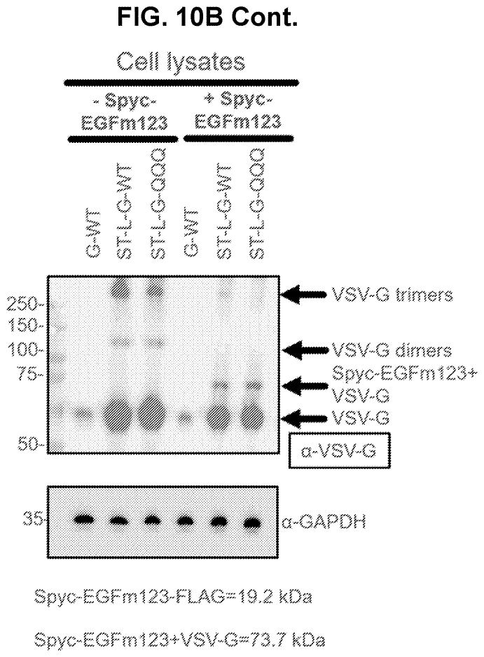

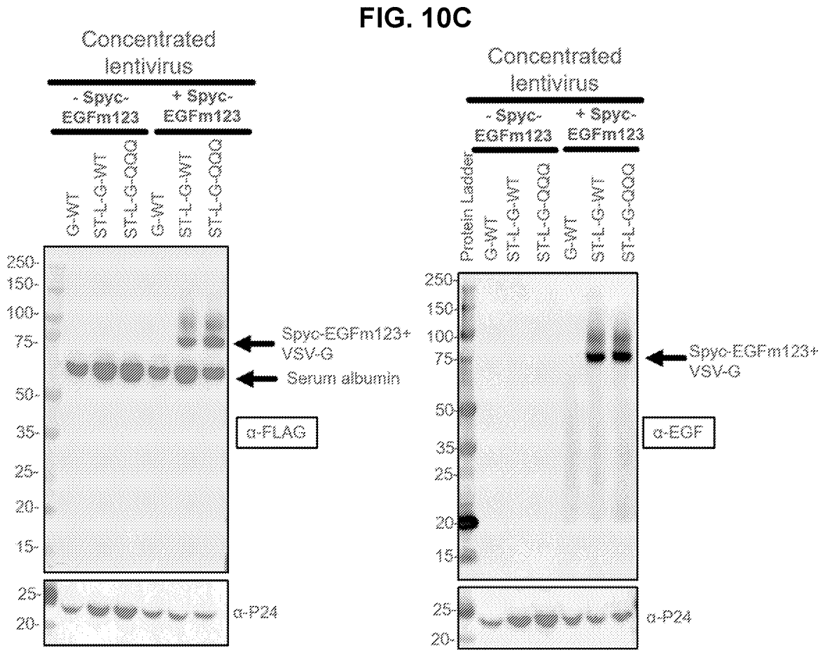

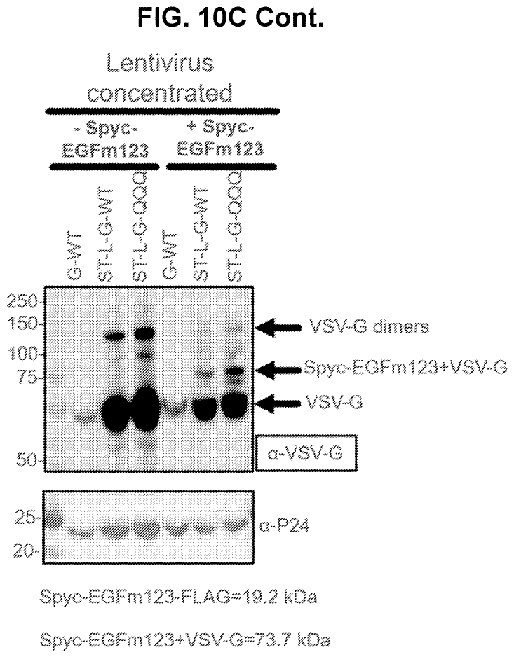

To test this method an experiment was designed as shown in FIG. 10A. HEK293T cells were cotransfected with the VSIV-G envelope expression plasmids without SpyTag as a negative control (VSV-G-WT) (SEQ ID NO: 96), the VSIV-G envelope expression plasmids with SpyTag inserted at site-1 with a linker (ST-L-VSV-G-WT) (SEQ ID NO: 85) or K47Q+R354Q+Y209Q substitution VSIV-G envelope expression plasmids with SpyTag inserted at site-1 with a linker (ST-L-VSV-G-QQQ) (SEQ ID NO: 93) with or without SpyCatcher-EGFm123-FLAG plasmid (SEQ ID NO: 99), along with the GFP lentivirus plasmid and the packaging plasmid. 24 hours after transfection cells were imaged using fluorescence microscopy to observe GFP expression. 72 hours after transfection the supernatant was collected, clarified, and passed through a 0.45 micron filter to collect the lentiviruses. Lentiviruses were then concentrated by Lenti-X concentrator. The lentiviruses were then used to transduce K562 cells.

Western blot analyses were performed on the cell lysates using anti-FLAG, anti-EGF or anti-VSIV-G antibodies (FIG. 10B). The sizes of proteins studied in this experiment were determined in kilodaltons (kDa), as shown in Table 4. The cell lysate results show bands at approximately 75 kDa, suggesting that covalent binding between the SpyCatcher-EGFm123 and VSIV-G-ST proteins occur inside the cells. Western blot analyses were also performed on the concentrated virus pellet with anti-FLAG, anti-EGF or anti-VSIV-G antibodies (FIG. 0C). The concentrated lentivirus also showed bands corresponding to covalently bound SpyCatcher-EGFm123-FLAG recombinant fusion proteins and SpyTag VSIV-G recombinant fusion proteins, suggesting that the SpyCatcher recombinant fusion protein binds to the SpyTag-VSIV-G on the lentivirus surface. Bands corresponding to the size of VSIV-G dimers and trimers were also observed suggesting SpyTag insertion and does not prevent the formation of VSIV-G dimers and trimers.

TABLE 4

| Protein | Size (kDa) |
|---|---|
| VSIV-G | 54.5 |
| EGFm123 | 5.38 |
| SpyCatcher | 12.4 |
| Spy Tag | 1.4 |
| GAPDH | 37 |
| P24 | 24 |
| SpyCatcher-EGFm123 | 19.2 |
| SpyCatcher-EGFm123-VSIV-G | 73.7 |

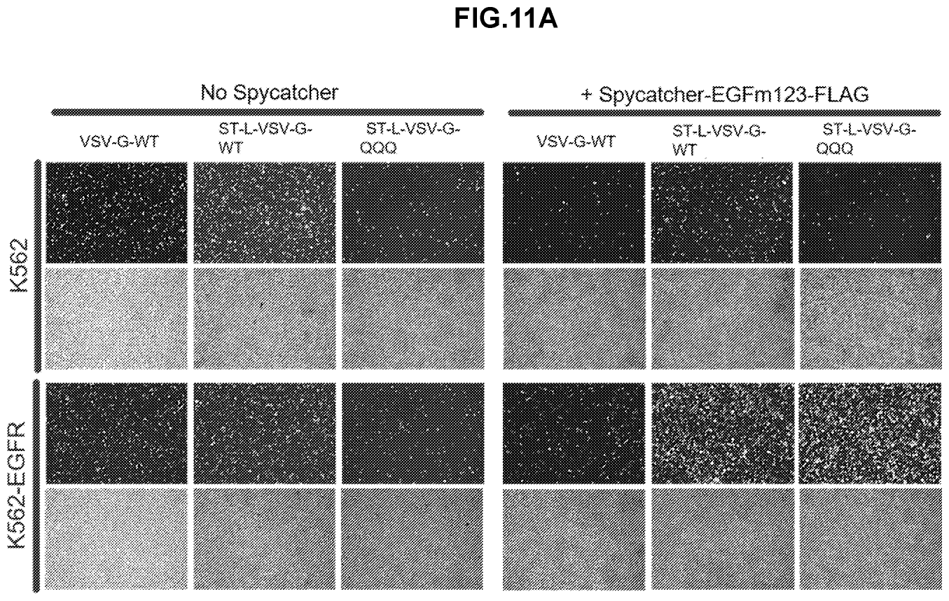

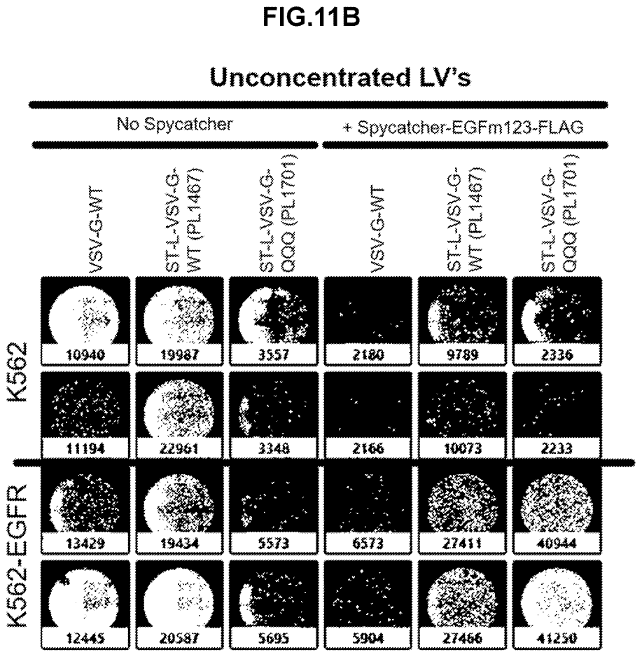

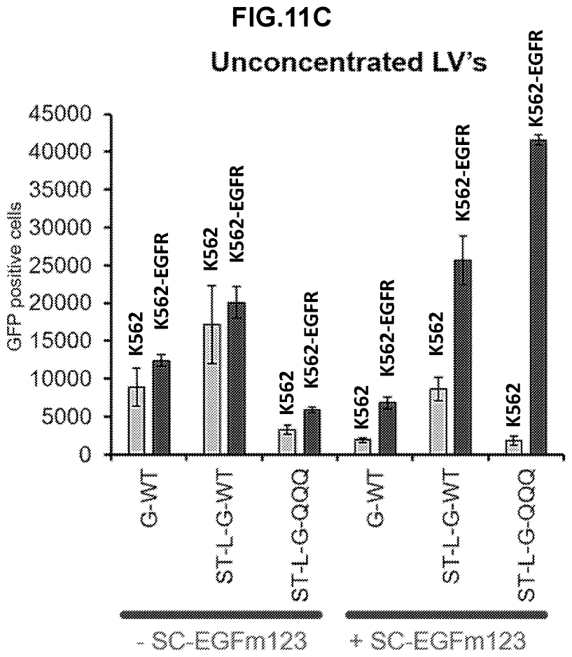

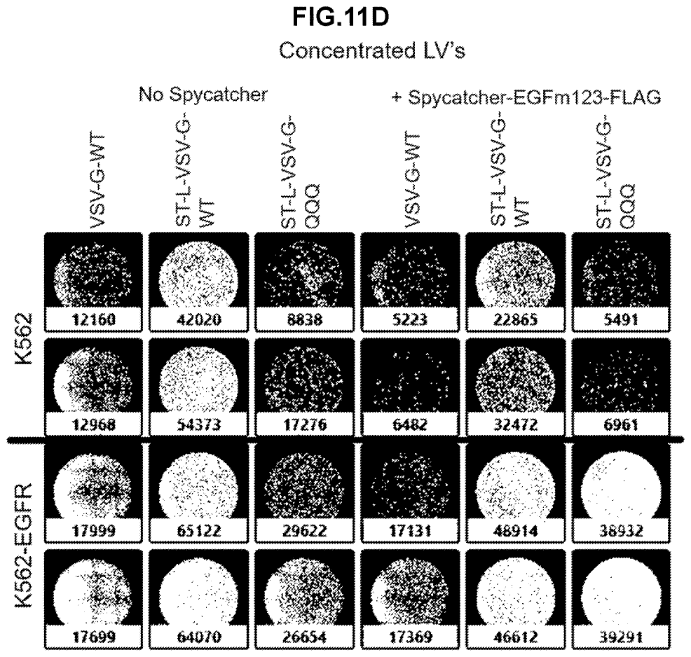

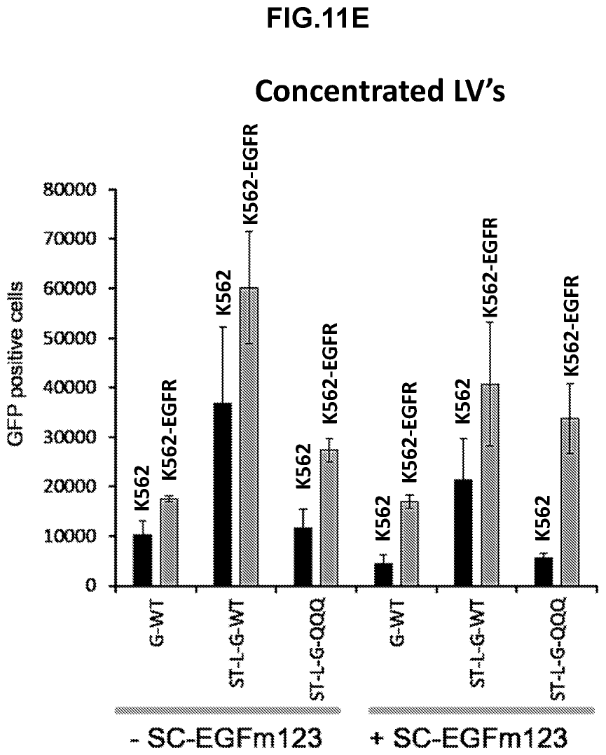

To test the ability of the SpyCatcher targeting molecule bound to SpyTag VSIV-G in lentivirus to target specific cell types for infection K562 cells, lacking the EGF receptor (EGFR), or K562 cells expressing the EGFR (K562-EGFR) were transduced with 20 ul of pseudotyped lentiviruses. The lentiviruses were pseudotyped with wild type VSIV-G without SpyTag (VSV-G-WT) (SEQ ID NO: 21), VSIV-G with SpyTag inserted at site-1 with a linker (ST-L-VSV-G-WT) (SEQ ID NO: 65), or K47Q+R354Q+Y209Q substitution VSIV-G with SpyTag inserted at site-1 with a linker (ST-L-VSV-G-QQQ) (SEQ ID NO: 66) with or without coexpressed SpyCatcher-EGFm123-FLAG (SEQ ID NO: 63). 72 hours after transduction the cells were imaged using fluorescent (top) and phase contrast (bottom) microscopy with a Nikon florescent microscope using a 10× objective (FIG. 11A). A CELIGO machine was used to image (FIG. 11B) and quantify the number of GFP positive cells (FIG. 11C) at 72 hours after transduction with unconcentrated lentivirus. These results suggest that the lentiviruses pseudotyped with the SpyTag VSIV-G bound to the SpyCatcher targeting molecule showed an increased transduction onto the K562-EGFR cells, particularly with the LDLR blinded VSIV-G with the K47Q+R354Q+Y209Q substitution, suggesting the specific targeting was mediated by the retargeted lentivirus. The same experiment was performed with concentrated lentivirus and similar results were observed (FIGS. 11D and 11E).

### Example 5

This example demonstrates the construction of targeted pseudotyped lentivirus particles by transfection of the SpyCatcher plasmid alone, collection of supernatant, and mixing via administration of that supernatant to cells transfected with the lentiviral, envelope, and packaging plasmids.

A method of mixing the SpyCatcher targeting molecule recombinant fusion protein together with the VSIV-G SpyTag envelope plasmid, lentiviral plasmid, and packaging plasmid was developed as shown in FIG. 9B.

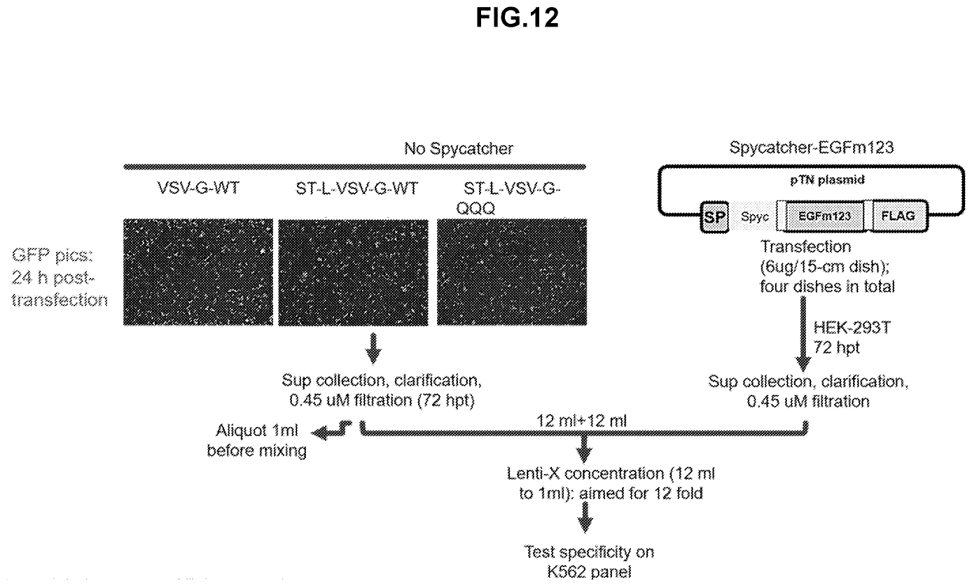

To test this method an experiment was designed as shown in FIG. 12. HEK-293T cells were transfected with cotransfected with the VSIV-G envelope expression plasmids without SpyTag as a negative control (VSV-G-WT) (SEQ ID NO: 96), the VSIV-G envelope expression plasmids with SpyTag inserted at site-1 with a linker (ST-L-VSV-G-WT) (SEQ ID NO: 85) or K47Q+R354Q+Y209Q substitution VSIV-G envelope expression plasmids with SpyTag inserted at site-1 with a linker (ST-L-VSV-G-QQQ) (SEQ ID NO: 93), along with the GFP lentivirus plasmid and the packaging plasmid. 24 hours after transfection cells were imaged using fluorescence microscopy to observe GFP expression. 72 hours after transfection the supernatant was collected, clarified, and passed through a 0.45 micron filter to collect the lentiviruses. In parallel HEK293T cells were transfected with the SpyCatcher-EGFm123-FLAG plasmid (SEQ ID NO: 99) at a concentration of 6 μg per 15 cm dish. 72 hours after transfection the supernatant was collected, clarified, and passed through a 0.45 micron filter to collect the targeting recombinant fusion protein, SpyCatcher-EGFm123-FLAG (SEQ ID NO: 65). The lentiviruses were then mixed with the targeting recombinant fusion protein and concentrated by Lenti-X concentrator. These lentiviruses were then used to transduce K562 cells.

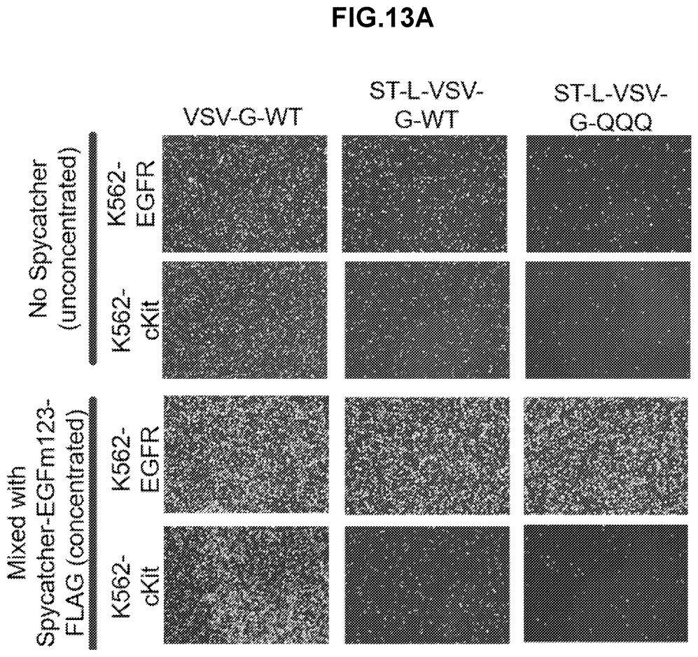

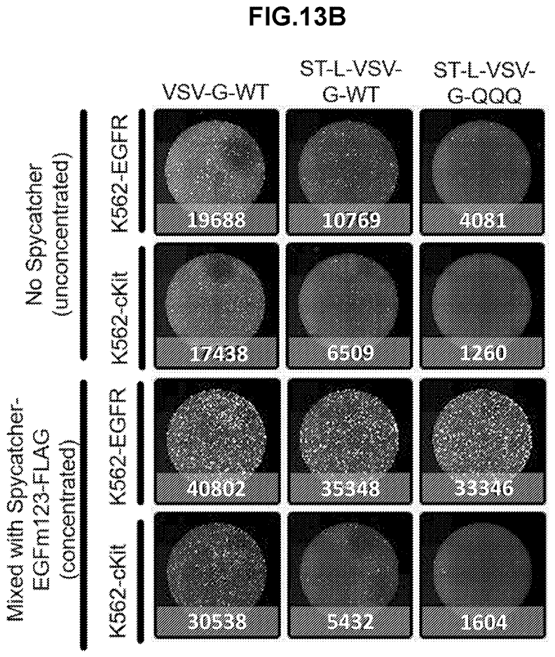

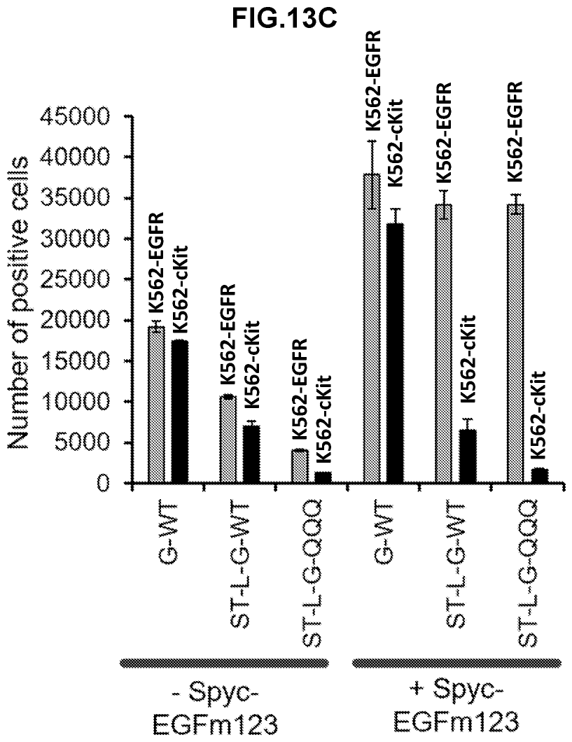

K562 cells expressing cKit, a stem cell factor (SCF) receptor, or EGFR were transduced with 10 ul of SpyTag VSIV-G pseudotyped lentiviruses alone or mixed with the SpyCatcher recombinant fusion protein. 72 hours after transduction the cells were imaged with a Nikon florescent microscope using a 10× objective (FIG. 13A). A CELIGO machine was used to image (FIG. 13B) and count the number of GFP positive cells (FIG. 13C) at 72 hours after transduction. The lentiviruses pseudotyped with SpyTag VSIV-G (ST-L-VSV-G-WT (SEQ ID NO: 85) and ST-L-VSV-G-QQQ (SEQ ID NO: 93)) that were mixed with the SpyCatcher recombinant fusion protein (Spycatcher-EGFm123-FLAG (SEQ ID NO: 65)) showed strong specificity for transducing the K562-EGFR cells compared to the K562-cKit cells, and those not mixed with the SpyCatcher recombinant fusion protein. These results suggest that the lentiviruses decorated with SpyTag VSIV-G and bound to a SpyCatcher targeting molecule recombinant fusion protein have the ability to selectively transduce specific cell types. This suggests the potential of this method for production of virions capable of delivering genetic cargo specifically to target cells in an in vivo setting.

### Example 6

This example demonstrates the ability of lentiviruses pseudotyped with SpyTag VSIV-G bound to a SpyCatcher recombinant fusion protein to infect specific cell types.

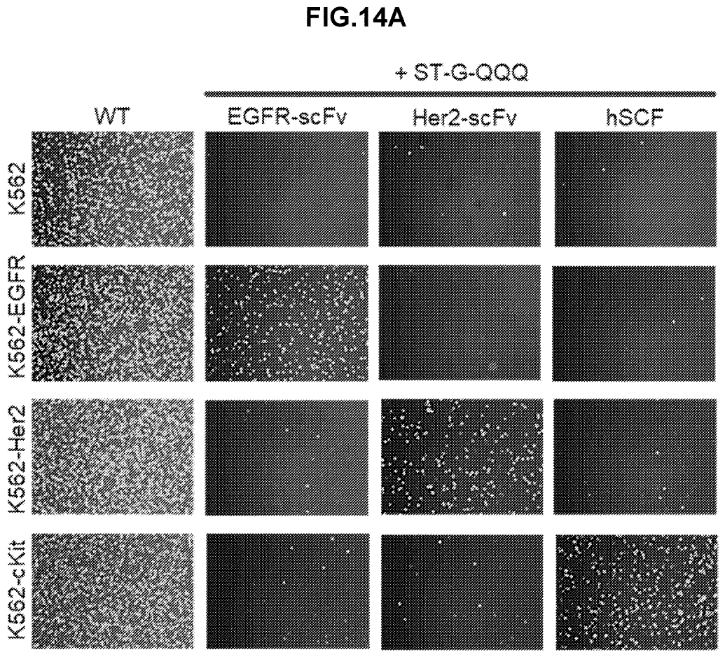

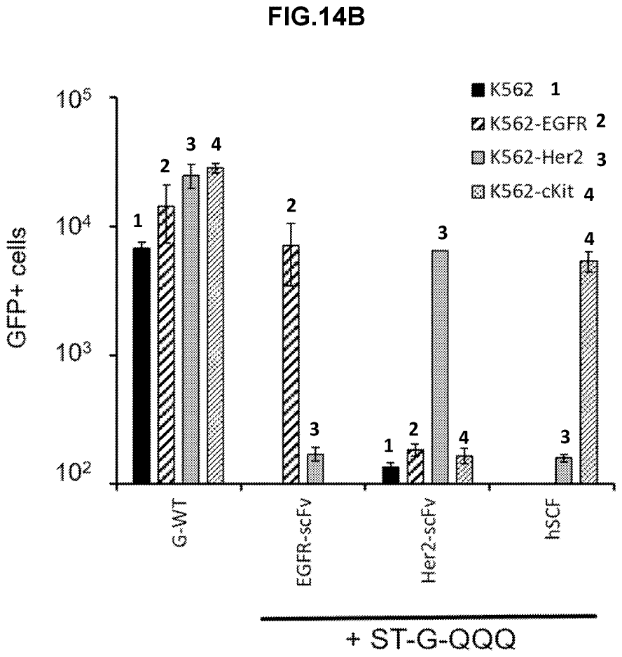

K562 cells innately express LDL receptor (LDLR). K562 cells, K562 cells expressing EGFR (K562-EGFR), K562 cells expressing Her2 (K562-Her2), and K562 cells expressing cKit (K562-cKit) were transduced with 10 μl of wild type VSIV-G (WT) (SEQ ID NO: 21) pseudotyped lentivirus or SpyTag VSIV-G-QQQ (ST-G-QQQ) (SEQ ID NO: 66) pseudotyped lentivirus with a coexpressed SpyCatcher (SEQ ID NO: 30) with EGF receptor single chain fragment variable (EGFR-scFv) (SEQ ID NO: 49), Her2 receptor scFv (Her2-scFv) (SEQ ID NO: 51), or human stem cell factor ligand (hSCF) (SEQ ID NO: 52) as the targeting molecule. 72 hours after transduction the cells were imaged with a Nikon florescent microscope using a 10× objective (FIG. 14A) and the number of GFP positive cells was quantified (FIG. 14B). All the K562 cells infected with the lentivirus pseudotyped with wild type VSIV-G showed counts of cells expressing GFP. This suggests that wild type VSIV-G decorated lentivirus were able to infect all the K562 cells by binding to LDLR and inducing membrane fusion. The K562 cells infected with the lentivirus pseudotyped with a SpyTag VSIV-G-QQQ coexpressing a SpyCatcher recombinant fusion protein showed very low counts of cells expressing GFP. This suggests that the LDLR blinded VSIV-G-QQQ has reduced binding affinity to LDLR and therefore has reduced infectivity in cells not expressing a binding partner for the targeting molecule. The K562 cells expressing EGFR infected with the lentivirus pseudotyped with a SpyTag VSIV-G-QQQ bound to a SpyCatcher only showed high counts of cells expressing GFP when the targeting molecule was EGFR-scFv (SEQ ID NO: 49). The K562 cells expressing Her2 infected with the lentivirus pseudotyped with a SpyTag VSIV-G-QQQ bound to a SpyCatcher only showed high counts of cells expressing GFP when the targeting molecule was Her2-scFv (SEQ ID NO: 51). The K562 cells expressing cKit infected with the lentivirus pseudotyped with a SpyTag VSIV-G-QQQ coexpressing a SpyCatcher only showed high counts of cells expressing GFP when the targeting molecule was hSCF (SEQ ID NO: 52). These results suggest that lentivirus pseudotyped with a SpyTag VSIV-G-QQQ coexpressing a SpyCatcher have reduced affinity for LDLR and specifically bind cells expressing surface proteins that the targeting molecule can bind. This suggests that this system could be used to target lentiviruses to infect specific cell types based on the expression of cell surface proteins. This suggests the potential of this method for production of virions capable of delivering genetic cargo specifically to target cells in an in vivo setting.

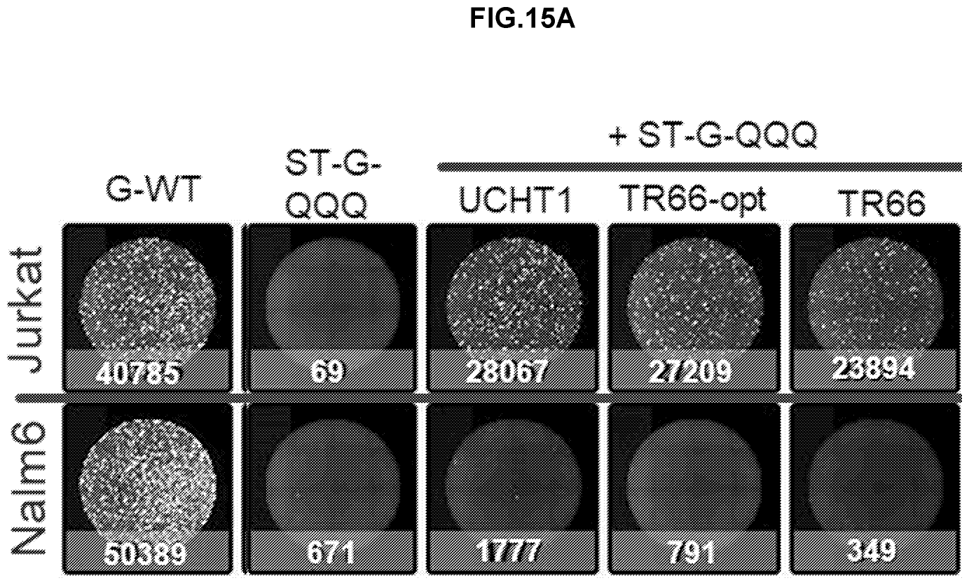

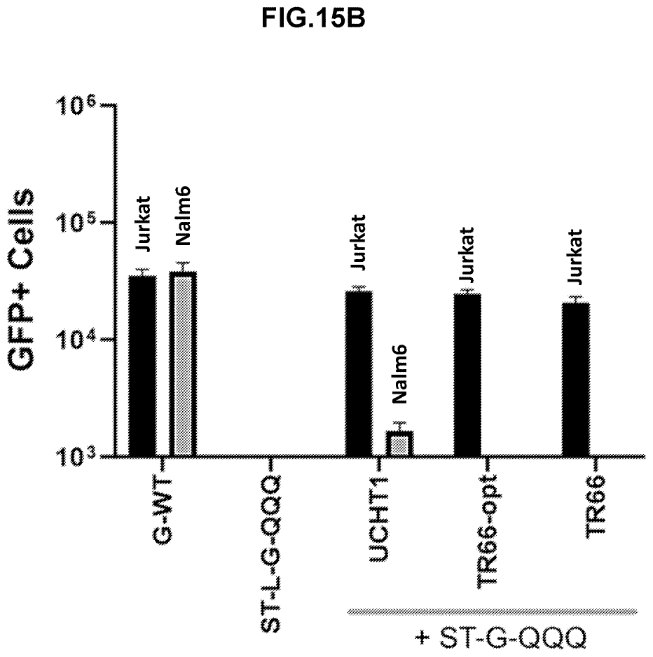

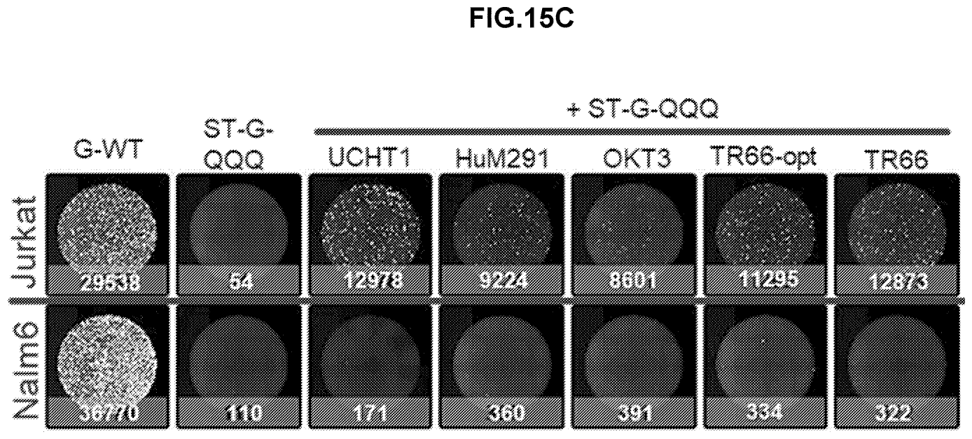

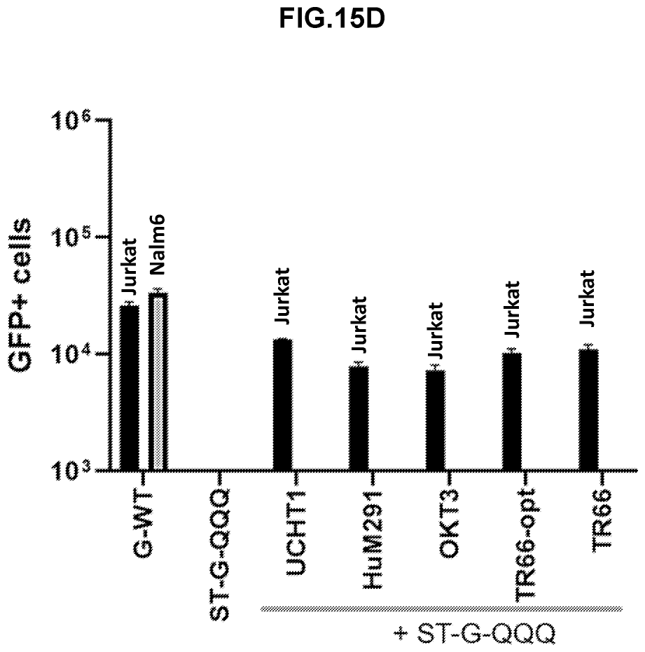

To test the specificity of lentiviruses pseudotyped with SpyTag VSIV-G bound to a SpyCatcher recombinant fusion protein to infect T cells further experiments were conducted using a T cell line (Jurkat) and B cell line (Nalm6). To target T cells, which express CD3, SpyCatcher constructs were designed with targeting molecules against CD3, including UCHT1 scFv (SEQ ID NO: 104), TR66 scFv (SEQ ID NO: 102), TR66-optimized scFv (SEQ ID NO: 103), HuM291 scFv (SEQ ID NO: 100), and OKT3 scFv (SEQ ID NO: 101). Lentiviruses were produced using the coexpression or mixing approach as previously described. Jurkat cells and Nalm6 cells were transduced with 20 μL of lentivirus pseudotyped with wild type VSIV-G (G-WT) (SEQ ID NO: 21), SpyTag VSIV-G-QQQ (ST-G-QQQ) (SEQ ID NO: 66) alone; or ST-G-QQQ (SEQ ID NO: 66) coexpressed with a SpyCatcher (SEQ ID NO: 30) with UCHT1 scFv (SEQ ID NOs: 45 and 46), TR66 scFv (SEQ ID NOs: 41 and 42), or TR66-optimized scFv (SEQ ID NOs: 43 and 44) targeting molecule; or ST-G-QQQ (SEQ ID NO: 66) mixed with SpyCatcher (SEQ ID NO: 30) with UCHT1 scFv (SEQ ID NOs: 45 and 46), HuM291 antibody (SEQ ID NOs: 37 and 38), OKT3 antibody (SEQ ID NOs: 39 and 40), TR66 scFv (SEQ ID NOs: 41 and 42), or TR66-optimized scFv (SEQ ID NOs: 43 and 44) targeting molecule. 48 hours after transduction with lentiviruses produced using the coexpression approach the cells were imaged with a Nikon florescent microscope using a 10× objective (FIG. 15A) and the number of GFP positive cells was quantified (FIG. 15B). 72 hours after transduction with lentiviruses produced using the mixing approach the cells were imaged with a Nikon florescent microscope using a 10× objective (FIG. 15C) and the number of GFP positive cells was quantified (FIG. 15D). Both the Jurkat and Nalm6 cells infected with the lentivirus pseudotyped with wild type VSIV-G (SEQ ID NO: 21) showed high counts of cells expressing GFP. This suggests that wild type VSIV-G decorated lentivirus were able to infect both Jurkat and Nalm6 cells by binding to LDLR and inducing membrane fusion. The Nalm6 cells infected with the lentivirus pseudotyped with a SpyTag VSIV-G-QQQ coexpressing or mixed with a SpyCatcher with a CD3 targeting molecule showed very low counts of cells expressing GFP. This suggests that the LDLR blinded VSIV-G-QQQ has reduced binding affinity to LDLR and therefore has reduced infectivity in cells not expressing CD3. The Jurkat cells infected with the lentivirus pseudotyped with a SpyTag VSIV-G-QQQ coexpressing or mixed with a SpyCatcher with a CD3 targeting molecule showed high counts of cells expressing GFP. This suggests that CD3 targeting molecule on the SpyCatcher is binding to surface CD3 on Jurkat cells and thereby initiating membrane fusion via the VSIV-G-QQQ. This further supports that this system could be used to target lentiviruses to infect specific cell types based on the expression of cell surface proteins. This also further supports the potential of this method for production of virions capable of delivering genetic cargo specifically to target cells in an in vivo setting with minimal background infection.

### Example 7

This example demonstrates the ability of lentiviruses pseudotyped with SpyTag VSIV-G bound to a SpyCatcher with CD3 targeting molecule to infect human peripheral blood mononuclear cells (PBMCs).

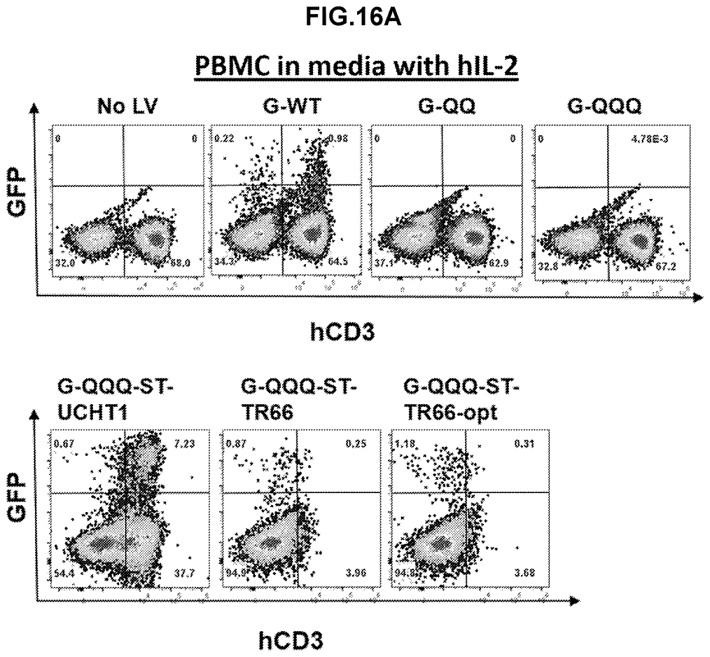

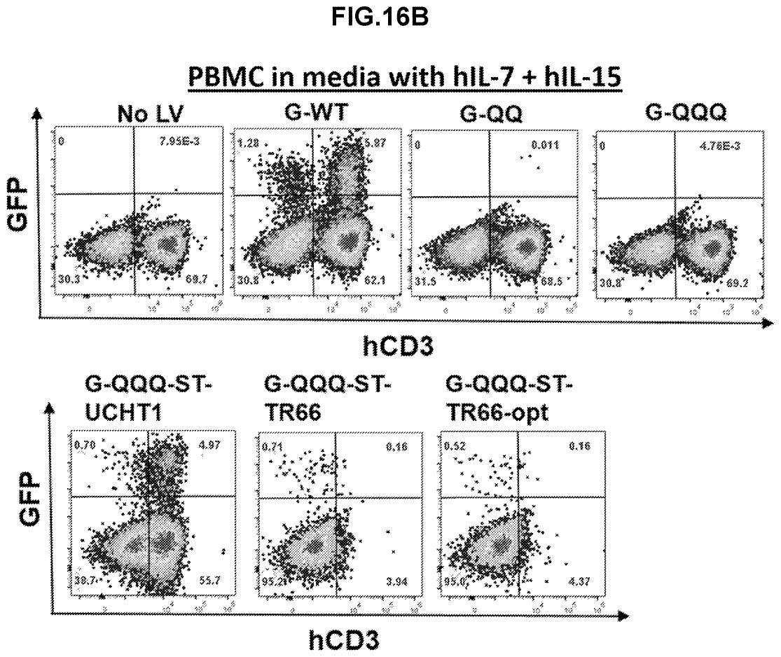

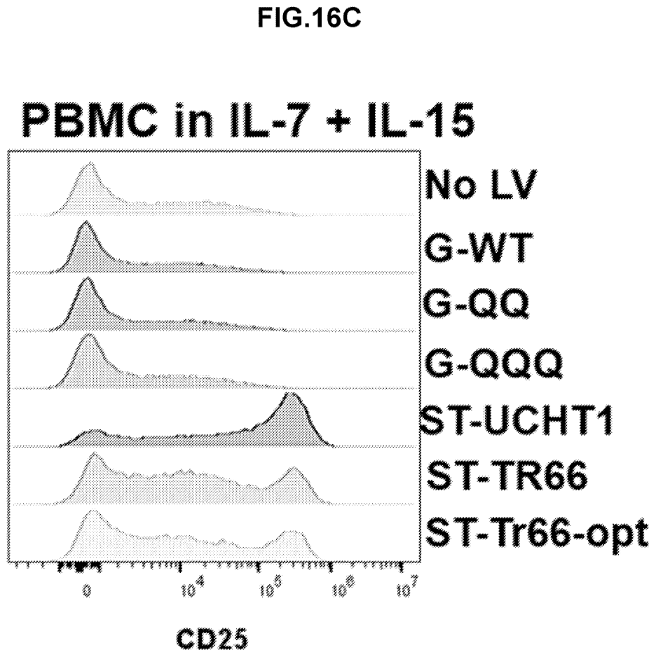

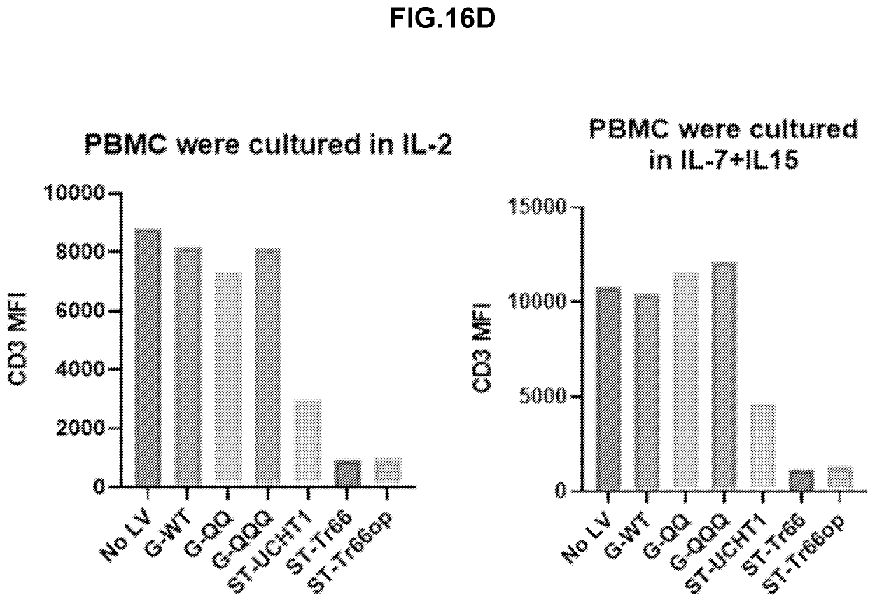

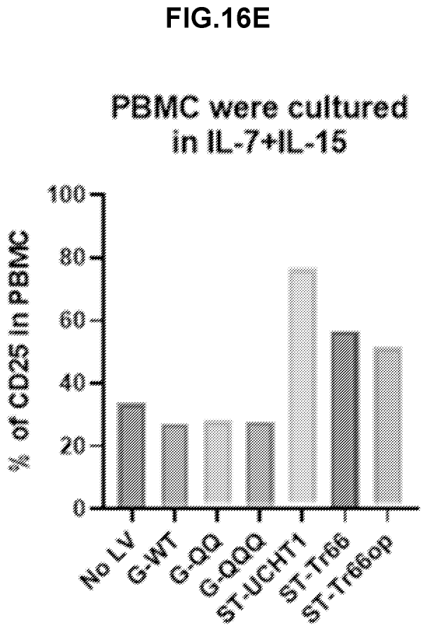

Whole blood samples were collected from healthy human subjects. PBMCs were then isolated using density gradient medium with Ficoll. Harvested PBMCs were rinsed with an appropriate buffer. PMBCs were then cultured in appropriate media containing either hIL-2 for T cell maintenance or hIL-7 and hIL-15 for T cell activation. The PBMCs were then infected with lentivirus pseudotyped with wild type VSIV-G (G-WT) (SEQ ID NO: 21), K47Q+R354Q substitution VSIV-G (G-QQ) (SEQ ID NO: 25), K47Q+R354Q+Y209Q substitution VSIV-G (G-QQQ) (SEQ ID NO: 26), or SpyTag K47Q+R354Q+Y209Q substitution VSIV-G (ST-G-QQQ) (SEQ ID NO: 66) alone or bound to SpyCatcher (SEQ ID NO: 30) with targeting molecule UCHT1 (G-QQQ-ST-UCHT1) (SEQ ID NOs: 45 and 46), TR66-opt (G-QQQ-ST-TR66-opt) (SEQ ID NOs: 43 and 44), or TR66 (G-QQQ-ST-TR66) (SEQ ID NOs: 41 and 42) at a multiplicity of infection (MOI) of 5. 3 days post infection the PBMCs cultured in media containing hIL-2 (FIG. 16A) or hIL-7 and hIL-15 (FIG. 16B) underwent flow analysis for human CD3 (hCD3), and the results were quantified (FIG. 16D). These results suggest that the lentiviruses pseudotyped with G-WT (SEQ ID NO: 21) and to a lesser extent G-QQ (SEQ ID NO: 25) and G-QQQ (SEQ ID NO: 26) infected most PBMCs, while those pseudotyped with G-QQQ-ST (SEQ ID NO: 66) and a CD3 targeting molecule infected specifically T cells in the PBMCs. This suggests that this system could be used in vivo to target specific cell populations, such as CD3 expressing T cells. 3 days post infection the PBMCs cultured in media containing hIL-7 and hIL-15 also underwent flow analysis for CD25 (FIG. 16C), and the results were quantified (FIG. 16E). The increased percentage of CD25 in PBMCs infected with the lentivirus pseudotyped with VSIV-G-QQQ bound to a SpyCatcher with a targeting molecule against CD3 compared to the other PBMC samples suggests that the infected T cells are activated. This suggests that this system could be used in vivo to activate T cells for treatments of diseases.

### Example 8

This example demonstrates methods of increasing the proportion of SpyTag VSIV-G bound to SpyCatcher and removing unbound SpyCatcher recombinant fusion protein using different concentration methods, mixing times, and third generation SpyTag and SpyCatcher.

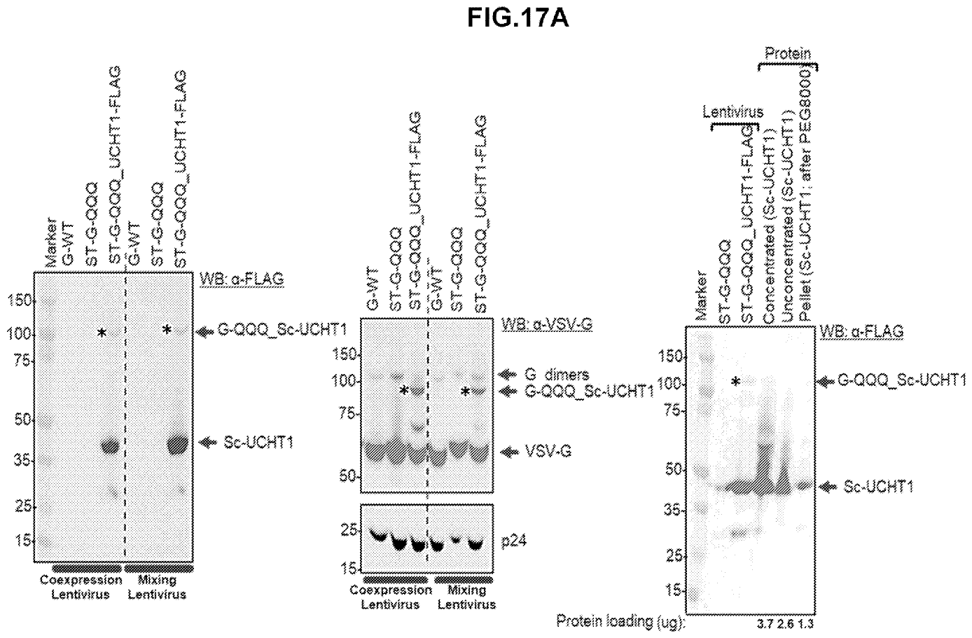

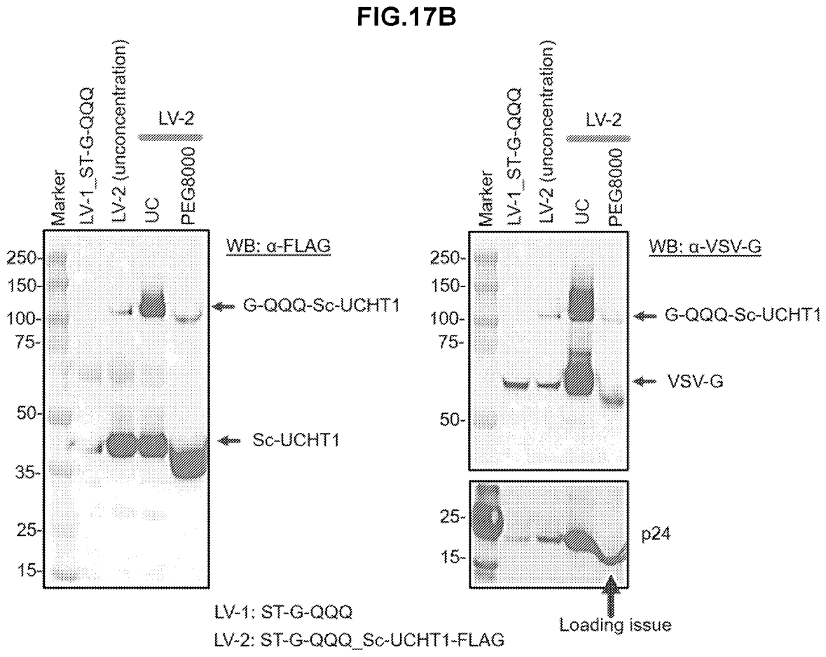

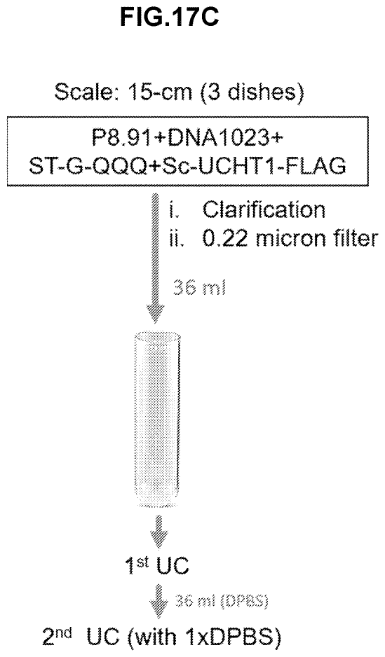

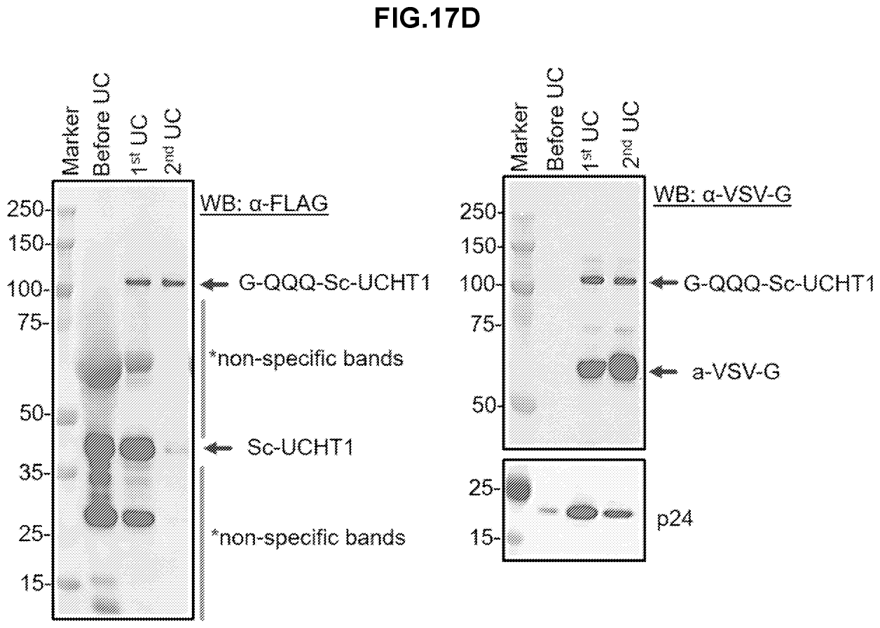

Experiments were performed to determine what percentage of SpyTag VSIV-G was bound to SpyCatcher in lentivirus. Lentivirus pseudotyped with VSIV-G (G-WT) (SEQ ID NO: 21), SpyTag K47Q+R354Q+Y209Q substitution G glycoprotein alone (ST-G-QQQ) (SEQ ID NO: 66) or with bound SpyCatcher with UCHT1 scFv and FLAG tag (SEQ ID NO: 67) (ST-G-QQQ_UCHT1-FLAG) were created using coexpression and mixing approaches as previously described. Lentiviruses were harvested from the producer cell culture supernatant and centrifuged at 800 relative centrifugal force (g) for 10 minutes at 4° C. and then the supernatant was filtered through 0.45 micron filter. The filtrate was then mixed at a 3:1 ratio with polyethylene glycol having an average molecular weight of 8,000 (PEG8000) obtained from SIGMA-ALDRICH®. The filtrate and PEG8000 solution was then shaken for 1 minute and incubated with rocking at 60 rotations per minute (rpm) for at least 4 hours at 4° C. and then spun down at 1600 g for 1 hour at 4° C. The supernatant is then removed and the viral pellet is resuspended. Unbound SpyCatcher recombinant fusion protein was precipitated by PEG8000 beside the virus particles. Western blot analysis was then performed on the lentiviruses against a FLAG antibody and a VSIV-G glycoprotein antibody (FIG. 17A). Based on visual analysis comparing the G-QQQ_Sc-UCHT1 bands to the VSV-G bands only about 10% of the SpyTag VSIV-G-QQQ were bound to SpyCatcher in lentivirus produced by coexpression or mixing approaches. Visual analysis comparing the G-QQQ_Sc-UCHT1 band to the Sc-UCHT1 band also revealed that most of the SpyCatcher recombinant fusion protein is unbound in lentiviruses produced by coexpression or mixing approaches. These results suggest that lentivirus pseudotyped with SpyTag VSIV-G-QQQ are able to successfully target cells expressing surface proteins even when only about 10% of the SpyTag VSIV-G-QQQ are bound to SpyCatcher with a targeting molecule.

To test the removal of unbound Spycatcher targeting molecules in the virus supernatants, ultracentrifugation was used to compare with PEG8000 concentration method. Lentivirus pseudotyped with SpyTag K47Q+R354Q+Y209Q substitution G glycoprotein alone (LV-1_ST-G-QQQ) (SEQ ID NO: 66) or with bound SpyCatcher with UCHT1 scFv and FLAG tag (SEQ ID NO: 67) (LV-2) were produced using the coexpression approach as previously described. Lentivirus pseudotyped with ST-G-QQQ-Sc-UCHT1-FLAG (LV-2) was collected and concentrated using ultracentrifugation (UC) or PEG8000. For ultracentrifugation concentration supernatant was collected and clarified by spinning at 2,000 rotations per minute (rpm) for 5 minutes and then passed through a 0.22 micron filter. The clarified supernatant was then loaded into ultracentrifuge clear tubes and spun at 27,100 rpm for 1.5 hours at 4° C., and in some protocols a second round of ultracentrifugation was performed. Western blot analysis was then performed on the lentiviruses against a FLAG antibody and a VSIV G glycoprotein antibody (FIG. 17B). Visual analysis of the G-QQQ-Sc-UCHT1 bands showed that the percentage of ST-G-QQQ bound to Sc-UCHT1 was clearly increased in the concentrated samples compared to unconcentrated and moreover, even higher than in the PEG8000 concentrated samples. A large amount of unbound SpyCatcher recombinant fusion protein was still observed in samples obtained using both ultracentrifugation and PEG8000 concentration methods.

To increase the percentage of SpyTag VSIV-G-QQQ (SEQ ID NO: 66) that were bound to SpyCatcher (SEQ ID NO: 30) in lentivirus and reduce the amount of unbound SpyCatcher recombinant fusion protein further tests of concentration methods using ultracentrifugation were performed as shown in the flowchart in FIG. 17C. Lentivirus pseudotyped with SpyTag K47Q+R354Q+Y209Q substitution G glycoprotein (SEQ ID NO: 66) with bound SpyCatcher with UCHT1 scFv and FLAG tag (SEQ ID NO: 67) (ST-G-QQQ_Sc-UCHT1-FLAG) were produced using the coexpression approach as previously described in three 15 cm dishes. Samples were then clarified and run through a 0.22 micron filter using 36 mL of wash. The sample was then concentrated by ultracentrifugation using 36 mL of DPBS and the pellet was resuspended in 1 mL. The sample was then concentrated by ultracentrifugation a second time using 1×DPBS and the pellet was resuspended in 1 mL. Western blot analysis was then performed on 20 μL of the unconcentrated (Before UC), first ultracentrifugation (1^{st} UC), and second ultracentrifugation (2^{nd} UC) samples of lentivirus against a FLAG antibody and a VSIV G glycoprotein antibody (FIG. 17D). Visual analysis of the G-QQQ-Sc-UCHT1 bands suggest that both 1 and 2 rounds of ultracentrifugation increased the percentage of ST-G-QQQ bound to Sc-UCHT1 compared to unconcentrated. Visual analysis of the Sc-UCHT1 band indicates that a second round of ultracentrifugation decreases the amount of unbound SpyCatcher recombinant fusion protein comparted to unconcentrated or only one ultracentrifugation. This suggests that concentration with two rounds of ultracentrifugation can increase the percentage of SpyTag rhabdoviral G glycoproteins bound to SpyCatcher with targeting molecules and decrease the amount of unbound SpyCatcher recombinant fusion protein.

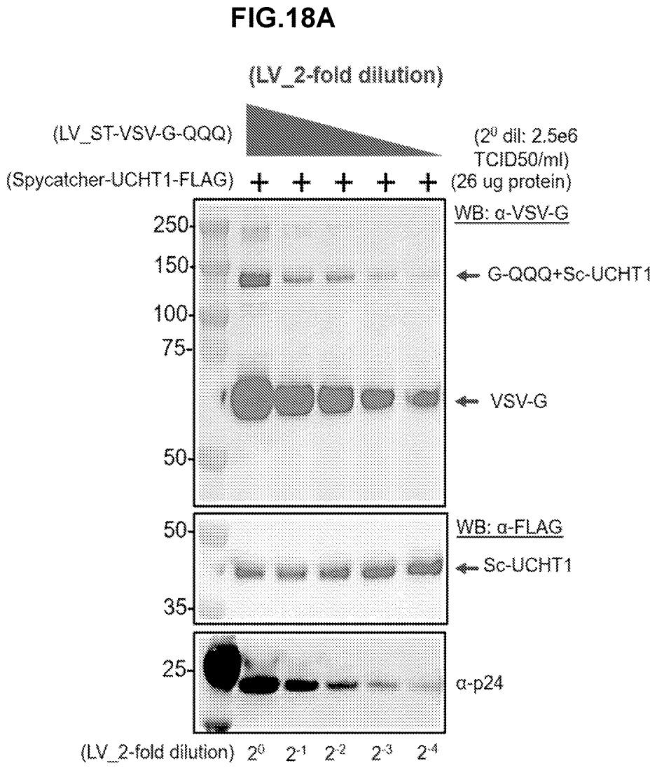

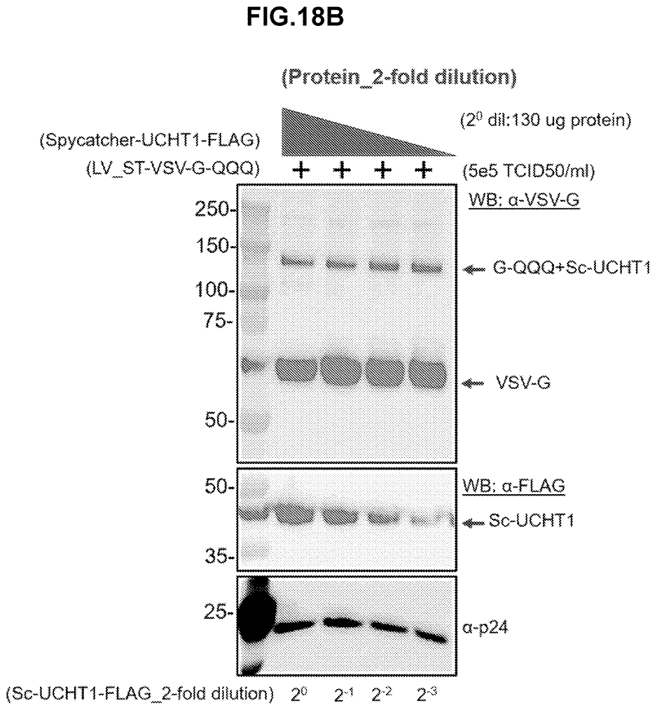

Experiments were performed to determine what effect the ratio of lentivirus pseudotyped with SpyTag rhabdoviral G glycoprotein to SpyCatcher with targeting molecule and the mixing time would have on the percentage of SpyTag VSIV-G recombinant fusion protein bound to SpyCatcher in lentivirus produced by the mixing approach dilution experiments were performed. Lentivirus pseudotyped with SpyTag K47Q+R354Q+Y209Q substitution G glycoprotein (SEQ ID NO: 66) (LV_ST-VSV-G-QQQ) and SpyCatcher with UCHT1 scFv and FLAG tag (Spycatcher-UCHT1-FLAG) (SEQ ID NO: 67) were produced separately using the mixing approach as previously described. 10 fold dilutions of LV_ST-VSV-G-QQQ starting from a concentration of 2.5e6 TCID50/mL (2⁰) were made and mixed with 26 μg of Spycatcher-UCHT1-FLAG. Western blot analysis was then performed on the samples against a FLAG antibody and a VSIV-G glycoprotein antibody (FIG. 18A). Visual comparison of the G-QQQ+Sc-UCHT1 and VSV-G bands suggest that the percentage of SpyTag VSIV-G recombinant fusion protein bound to SpyCatcher in lentivirus was not affected by changing the ratio of lentivirus pseudotyped with SpyTag rhabdoviral G glycoprotein recombinant fusion protein to SpyCatcher with targeting molecule recombinant fusion protein. Another dilution experiment was performed using the mixing approach previously described, but with 10 fold dilutions of Spycatcher-UCHT1-FLAG starting from 130 μg (2⁰) mixed with LV_ST-VSV-G-QQQ at a concentration of 5e5 TCID50/mL. Western blot analysis was then performed on the samples against a FLAG antibody and a VSIV-G glycoprotein antibody (FIG. 18B1). Visual analysis of the bands showed no obvious effect on the percentage of SpyTag VSIV-G recombinant fusion protein bound to SpyCatcher recombinant fusion protein in lentivirus.

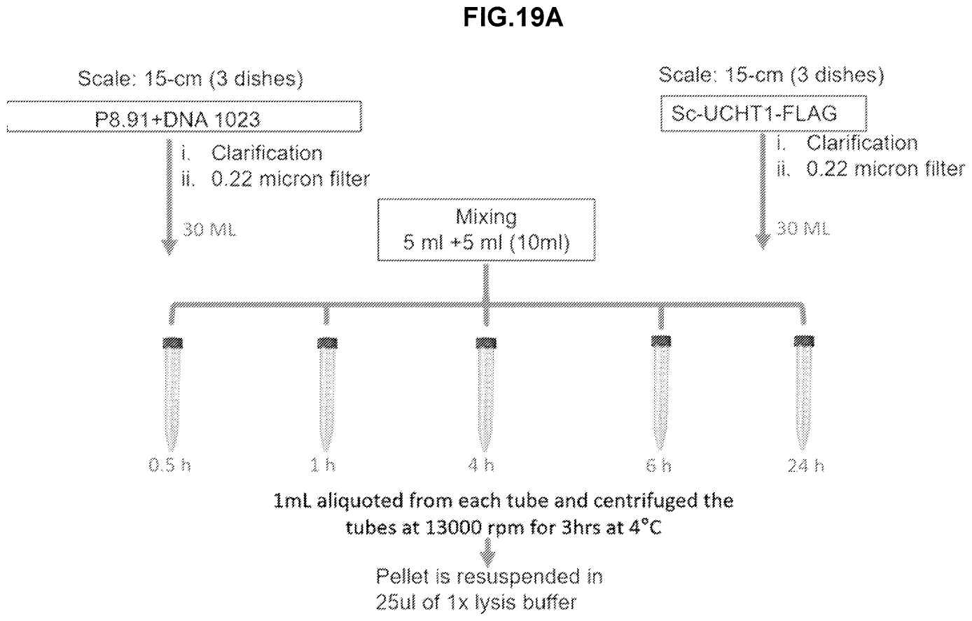

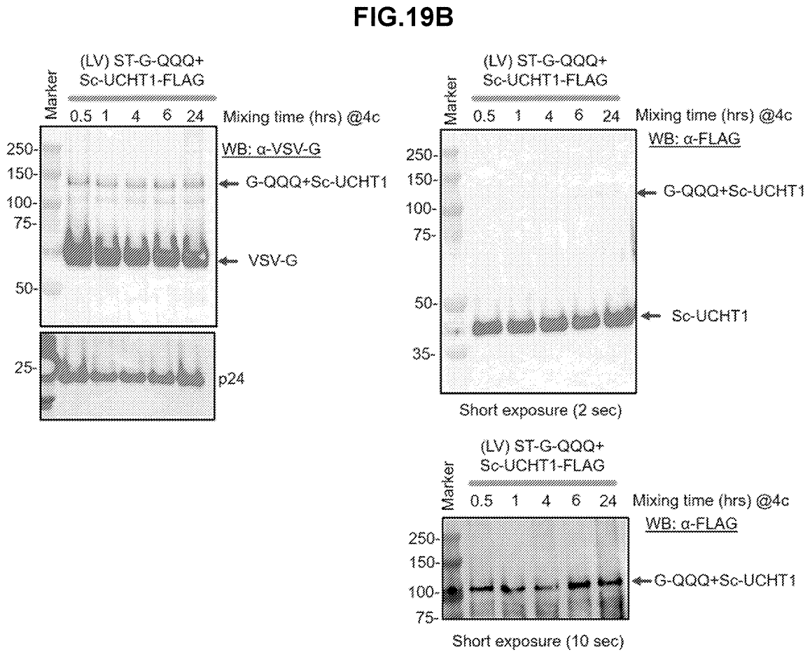

Experiments to determine the effect of mixing time on the percentage of SpyTag VSIV-G recombinant fusion protein bound to SpyCatcher recombinant fusion protein in lentivirus were performed as shown in the flowchart of FIG. 19A. Lentivirus pseudotyped with SpyTag K47Q+R354Q+Y209Q substitution G glycoprotein (SEQ ID NO: 66) (LV_ST-VSV-G-QQQ) and SpyCatcher with UCHT1 scFv and FLAG tag (Spycatcher-UCHT1-FLAG) (SEQ ID NO: 67) were produced separately using the mixing approach as previously described in three 15 cm dishes. Samples were then clarified and run through a 0.22 micron filter using 30 mL of wash. 5 mL of the LV-ST-G-QQQ were mixed with 5 mL of the Sc-UCHT1-FLAG for 0.5, 1, 4, 6, or 24 hours at room temperature on a rocker. After the mixing time was complete 1 mL of each sample was centrifuged at 13000 rpm for 3 hours at 4° C., and the resulting pellet was resuspend in 25 μL of 1× lysis buffer. Western blot analysis was then performed on the samples against a FLAG antibody and a VSIV-G glycoprotein antibody with short exposure of 2 or 10 seconds (FIG. 19B). Visual analysis of the western blots suggest that the percentage of SpyTag VSIV-G recombinant fusion protein bound to SpyCatcher recombinant fusion protein in lentivirus was not affected by additional mixing time beyond 0.5 hours. This suggests that the binding of SpyTag VSIV-G recombinant fusion protein to SpyCatcher recombinant fusion protein in lentivirus occurs in less than 0.5 hour.

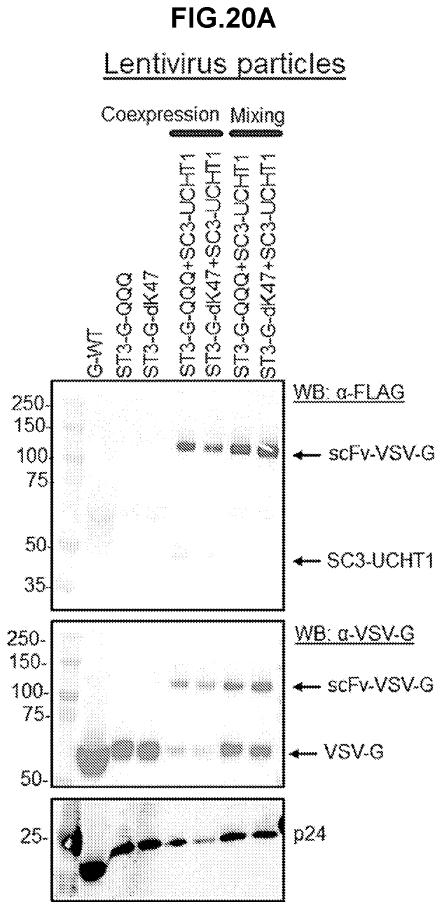

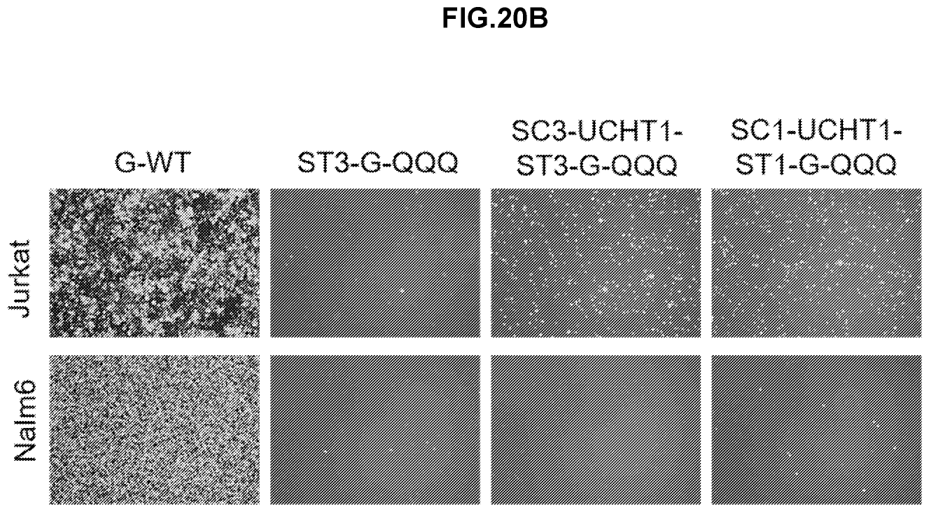

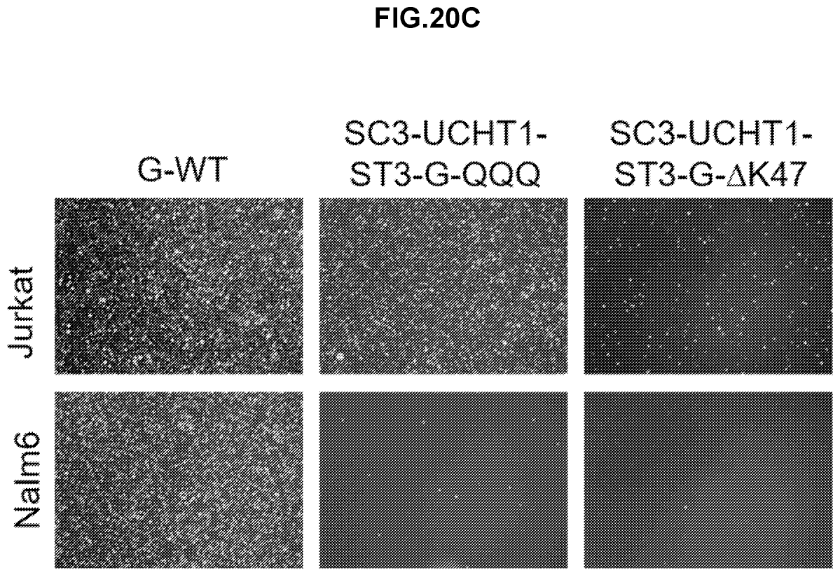

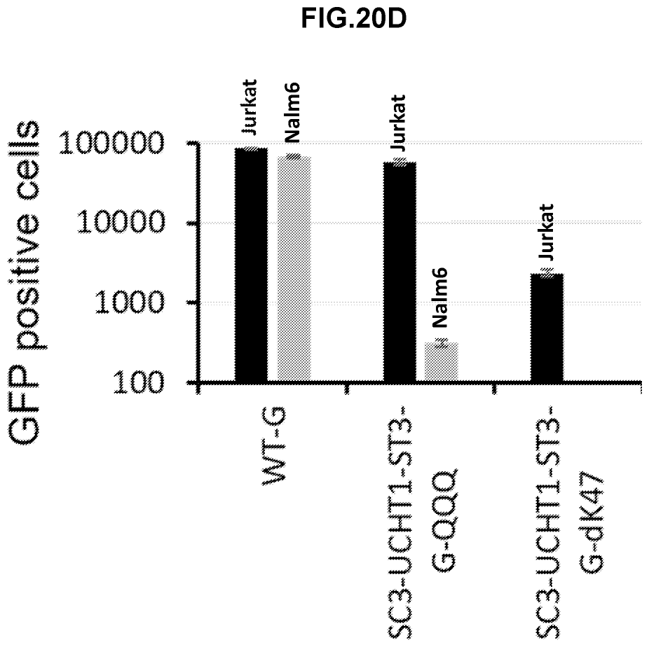

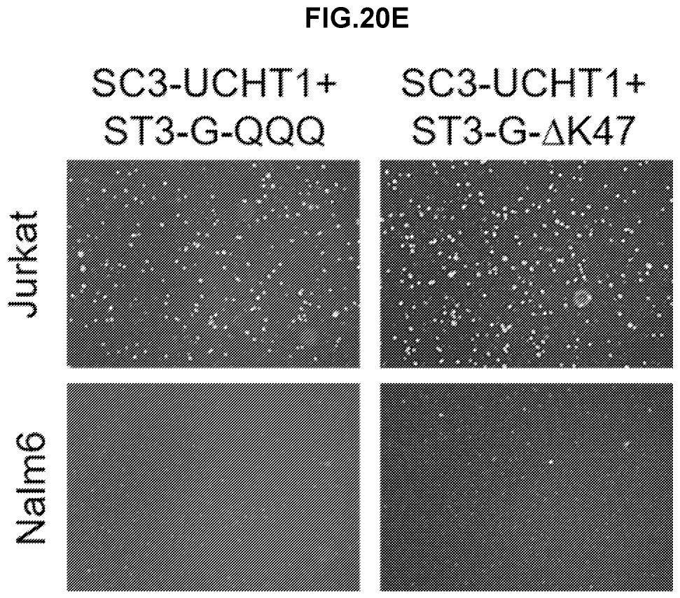

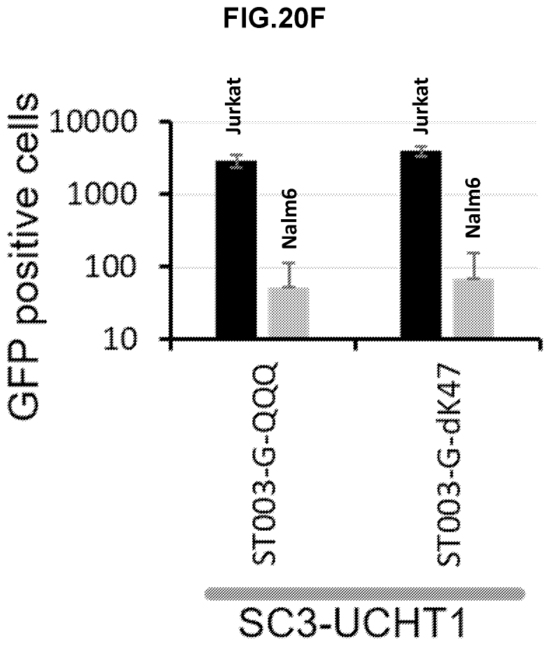

To increase the percentage of SpyTag VSIV-G-QQQ recombinant fusion protein bound to SpyCatcher recombinant fusion protein in lentivirus new plasmids using third generation SpyTag and SpyCatcher were designed and tested. New plasmids for lentivirus production were designed replacing the first generation SpyTag, SpyTag001 (SEQ ID NO: 1), and first generation SpyCatcher, SpyCatcher001 (SEQ ID NO: 30), with third generation SpyTag, SpyTag003 (SEQ ID NO: 3), and third generation SpyCatcher, SpyCatcher 003 (SEQ ID NO: 32), respectively. Plasmids encoding third generation SpyTag fused to a K47Q+R354Q+Y209Q substitution G glycoprotein (SEQ ID NO: 94), third generation SpyTag fused to a K47 deletion G glycoprotein (SEQ ID NO: 95), third generation SpyCatcher fused to a TR66-opt scFv and FLAG tag (SEQ ID NO: 114), third generation SpyCatcher fused to a UCHT1 scFv and FLAG tag (SEQ ID NO: 115), third generation SpyCatcher fused to a hSCF ligand and FLAG tag (SEQ ID NO: 116), and third generation SpyCatcher fused to a mSCF and FLAG tag (SEQ ID NO: 117) were created. Lentiviruses pseudotyped with VSIV-G glycoprotein (G-WT) (SEQ ID NO: 21), third generation SpyTag K47Q+R354Q+Y209Q substitution G glycoprotein alone (ST3-G-QQQ) (SEQ ID NO: 68) or bound to third generation SpyCatcher with UCHT1 scFV and FLAG tag (SEQ ID NO: 69) (ST3-G-QQQ+SC3-UCHT1), third generation SpyTag K47 deletion G glycoprotein alone (ST3-G-ΔK47) (SEQ ID NO: 70) or bound to third generation SpyCatcher with UCHT1 scFV and FLAG tag (SEQ ID NO: 69) (ST3-G-ΔK47+SC3-UCHT1) were produced using the coexpression as previously described or mixing approach with 30 minutes of mixing at room temperature followed by 1 hour at 4° C. and then an ultracentrifugation step as previously described. The G-WT lentivirus was concentrated using the PEG8000 method previously described, and all of the other lentivirus samples were concentrated with two rounds of ultracentrifugation followed by resuspension of the pellet in 1× DPBS as previously described. Western blot analysis was then performed on 10 μL of each sample against a FLAG antibody and a VSIV-G glycoprotein antibody (FIG. 20A). Visual comparison of the scFv-VSV-G and VSV-G bands suggest that about 50% of the ST3-G-QQQ bound to SC3-UCHT1-FLAG in lentivirus. This suggests that third generation SpyTag and SpyCatcher may show improved specificity in targeting and increased infectivity. Visual analysis of the SC3-UCHT1 band also suggests that there are low amounts of unbound SpyCatcher recombinant fusion protein. This suggests that use of the third generation SpyTag and SpyCatcher could improve the efficacy of this system in vivo for targeting specific cell populations and reducing levels of unbound SpyCatcher targeting molecules proteins.

To compare the targeting specificity of first and third generation SpyTag and SpyCatcher Jurkat and Nalm6 cells were infected with 10 μL of lentivirus pseudotyped with VSIV-G glycoprotein (G-WT) (SEQ ID NO: 21), third generation SpyTag K47Q+R354Q+Y209Q substitution G glycoprotein alone (ST3-G-QQQ) (SEQ ID NO: 68) or bound to third generation SpyCatcher with UCHT1 scFV and FLAG tag (SEQ ID NO: 69) (SC3-UCHT1-ST3-G-QQQ), or first generation SpyTag K47Q+R354Q+Y209Q substitution G glycoprotein (SEQ ID NO: 660 bound to first generation SpyCatcher with UCHT1 scFV and FLAG tag (SEQ ID NO: 67) (SC3-UCHT1-ST1-G-QQQ) produced by the coexpression approach as previously described. After transduction, fluorescent micrographs were captured by Nikon microscope at 10× objective lens with 50 ms exposure for cells infected with G-WT lentivirus and 500 ms exposures for all the other cells (FIG. 20B). While these lentiviruses were produced in different batches and the transduction was based on lentivirus volume the results suggest that in this system third generation SpyTag and SpyCatcher has similarly effective binding specificity to the first generation. The low number of infected Nalm6 cells infected by SC3-UCHT1-ST3-G-QQQ also suggests that third generation SpyTag and SpyCatcher may reduce off target binding.

Additional testing of the third generation SpyTag and SpyCatcher was performed comparing LDLR blinding mutations in VSIV-G and the coexpression and mixing approach to lentivirus production. Jurkat and Nalm6 cells were seeded at 5×10⁴ cells/well and then infected with 5×10⁷ (estimated MOI=10) lentivirus particles pseudotyped with VSIV-G glycoprotein (G-WT) (SEQ ID NO: 21), third generation SpyTag K47Q+R354Q+Y209Q substitution G glycoprotein (SEQ ID NO: 68) bound to third generation SpyCatcher with UCHT1 scFv and FLAG tag (SEQ ID NO: 69) (ST3-G-QQQ+SC3-UCHT1), or third generation SpyTag K47 deletion G glycoprotein (SEQ ID NO: 70) bound to third generation SpyCatcher with UCHT1 scFv and FLAG tag (SEQ ID NO: 70) (SC3-UCHT1+ST3-G-ΔK47) produced by the coexpression approach as previously described. 120 hours post transduction, fluorescent micrographs were captured by Nikon microscope at 10× objective lens with 50 ms exposure for cells infected with G-WT lentivirus and 300 ms exposures for all the other cells (FIG. 20C) and a CELIGO machine was used to quantify the number of GFP positive cells (FIG. 20D). A similar experiment was performed in Jurkat and Nalm6 cells that were seeded at 5×10⁴ cells/well and then infected with 5×10⁷ lentivirus particles pseudotyped with third generation SpyTag K47Q+R354Q+Y209Q substitution G glycoprotein (SEQ ID NO: 68) bound to third generation SpyCatcher with UCHT1 scFV and FLAG tag (SEQ ID NO: 69) (ST3-G-QQQ+SC3-UCHT1) or 2.5×10⁸ lentivirus particles pseudotyped with third generation SpyTag K47 deletion G glycoprotein (SEQ ID NO: 70) bound to third generation SpyCatcher with UCHT1 scFv and FLAG tag (SEQ ID NO: 69) (SC3-UCHT1+ST3-G-ΔK47) produced by the mixing approach as previously described. 120 hours post transduction, fluorescent micrographs were captured by Nikon microscope at 10× objective lens with 300 ms exposures (FIG. 20E) and a CELIGO machine was used to quantify the number of GFP positive cells (FIG. 20F). The results of these experiments suggest that third generation SpyTag and SpyCatcher demonstrate targeting specificity in this system. The low number of Nalm6 cells infected by the lentivirus pseudotyped with SC3-UCHT1-ST3-G-ΔK47 also suggests that the K47 deletion G glycoprotein (SEQ ID NO: 27) reduces LDLR binding and off target infection even more than the K47Q+R354Q+Y209Q substitution G glycoprotein (SEQ ID NO: 26), at least when producing lentivirus using the coexpression approach.

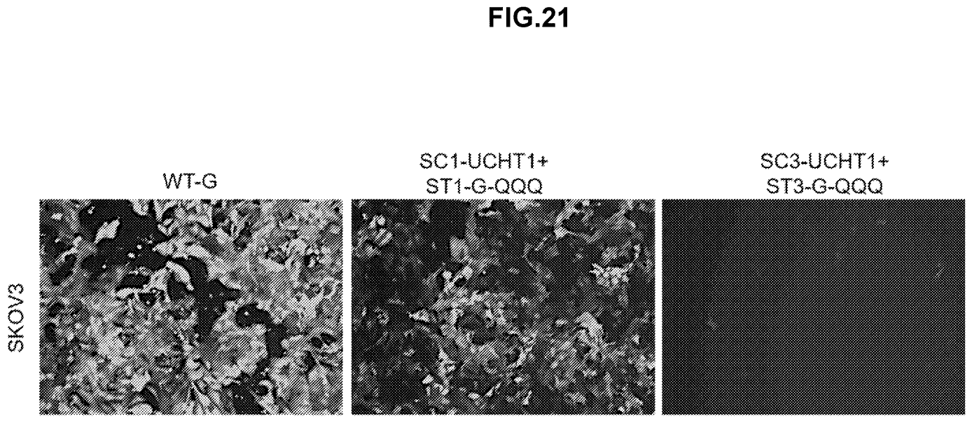

First generation SpyTag and SpyCatcher are known to have non-specific interactions with cells from a human ovarian cancer line (SKOV3). To compare off target binding in this system SKOV3 cells were seeded at 1×10⁴ cells/well and then infected with 1×10⁶ lentivirus particles pseudotyped with VSIV-G glycoprotein (G-WT) (SEQ ID NO: 21), third generation SpyTag K47Q+R354Q+Y209Q substitution G glycoprotein bound (SEQ ID NO: 68) to third generation SpyCatcher with UCHT1 scFV and FLAG tag (SEQ ID NO: 69) (ST3-G-QQQ+SC3-UCHT1), or first generation SpyTag K47Q+R354Q+Y209Q substitution G glycoprotein (SEQ ID NO: 70) bound to first generation SpyCatcher with UCHT1 scFv and FLAG tag (SEQ ID NO: 69) (SC1-UCHT1+ST1-G-QQQ). 120 hours post transduction, fluorescent micrographs were captured by Nikon microscope at 10× objective lens with 30 ms exposure for the WT-G cells and 100 ms exposure for the other cells (FIG. 21). These results suggest that while lentiviruses pseudotyped with first generation SpyTag and SpyCatcher in this system can have off target interactions, use of third generation SpyTag and SpyCatcher reduce or abolish this off target interaction. This suggests that lentiviruses pseudotyped with third generation SpyTag VSIV-G-QQQ bound to SpyCatcher with a targeting molecule could be used in vivo for genetic delivery to specific cell targets with minimal off target infection.

### Example 9

This example demonstrates the targeting specificity of lentivirus pseudotyped with alternative rhabdoviral G glycoproteins compared to VSIV-G.

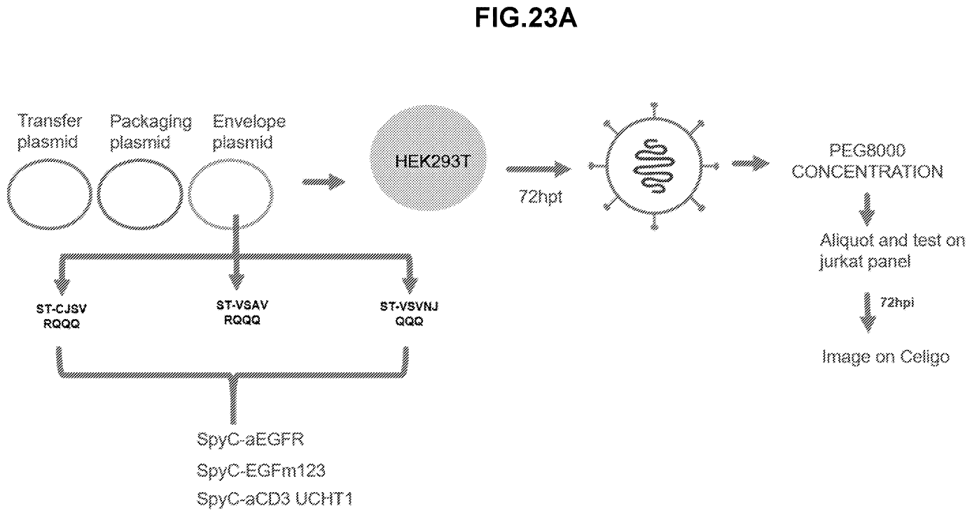

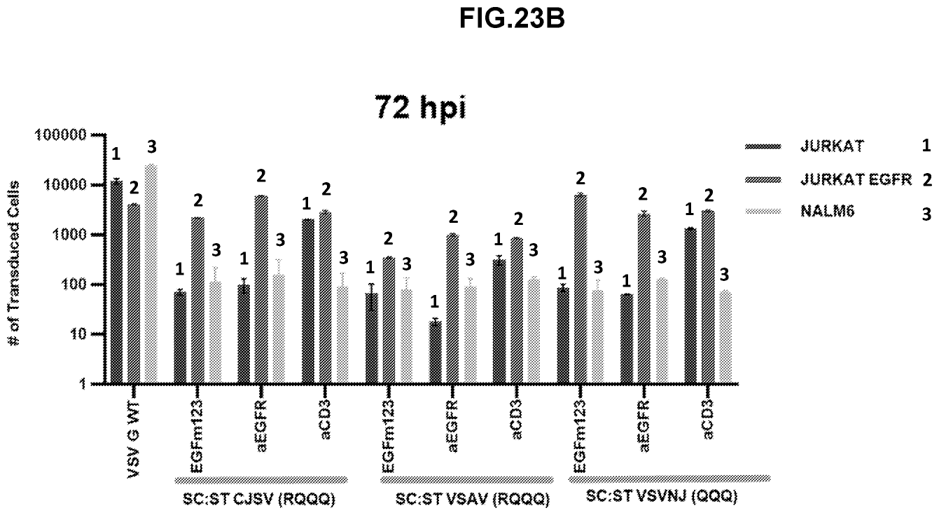

In order to test the targeting specificity of alternative rhabdoviral G glycoproteins compared to VSIV-G other Rhabdoviruses were considered (FIGS. 22A and 22B). Three alternative rhabdoviruses from Vesiculovirus genus were selected for further testing, Vesiculovirus newjersey (VSVNJ), Vesiculovirus Alagoas (VSAV) with a K1R substitution (SEQ ID NO: 124) or a G115A substitution (SEQ ID NO: 125), and Vesiculovirus Carajas (VSCV) with a K1R substitution (SEQ ID NO: 126) or a G115A substitution (SEQ ID NO: 127). The experiment was performed as indicated in the flowchart shown in FIG. 23A, and lentiviruses were produced using the previously described mixing approach. A VSCV-G plasmid (SEQ ID NO: 12)) encoding a VSCV signal peptide (SP) (SEQ ID NO: 123) N terminal to a SpyTag (SEQ ID NO: 1) fused with K1R+K47Q+R354Q+Y209Q substitution VSCV-G (SEQ ID NO: 71) via a linker (SEQ ID NO: 12), a VSAV-G plasmid (SEQ ID NO: 119) encoding a VSAV SP (SEQ ID NO: 122) N terminal to SpyTag (SEQ ID NO: 1) fused with K1R+K47Q+R354Q+Y209Q substitution VSAV-G (SEQ ID NO: 72) via a linker (SEQ ID NO: 12), and a VSVNJ-G plasmid (SEQ ID NO: 118) encoding a VSVNJ SP (SEQ ID NO: 121) N terminal to SpyTag (SEQ ID NO: 1) fused with K47Q+R354Q+Y209Q substitution VSVNJ-G (SEQ ID NO: 118) via a linker (SEQ ID NO: 12) were created. The number of Jurkat cells, Jurkat cells expressing EGFR, and Nalm6 cells transduced with lentivirus pseudotyped with wild type VSIV-G without SpyTag (VSV G WT) (SEQ ID NO: 21), and lentivirus pseudotyped with SpyTag K47Q+R354Q+Y209Q substitution CJSV-G (ST CJSV (RQQQ)) (SEQ ID NO: 74), SpyTag K47Q+R354Q+Y209Q substitution VSAV-G (ST VSAV (RQQQ)) (SEQ ID NO: 75), or SpyTag K47Q+R354Q+Y209Q substitution VSVNJ-G (ST VSVNJ (QQQ)) (SEQ ID NO: 73) bound to a recombinant fusion protein of SpyCatcher (SEQ ID NO: 30) fused with EGFm123 (SEQ ID NO: 59), EGFR scFV (aEGFR) (SEQ ID NO: 49), or UCHT1 scFv (aCD3) (SEQ ID NOs: 45 and 46) as a targeting molecule was quantified using a logarithmic scale (FIG. 23B). A higher number of Jurkat cells expressing EGFR were transduced by lentiviruses pseudotyped by ST CJSV (RQQQ) (SEQ ID NO: 74), ST VSAV (RQQQ) (SEQ ID NO: 75), or ST VSVNJ (QQQ) (SEQ ID NO: 73) bound to a recombinant fusion protein of SpyCatcher fused with either EGFm123 (SEQ ID NO: 59) or EGFR scFv (SEQ ID NO: 49) as targeting molecule compared to the number of transduced Jurkat and Nalm6 cells. A higher number of Jurkat cells and Jurkat cells expressing EGFR were transduced by lentiviruses pseudotyped by ST CJSV (RQQQ) (SEQ ID NO: 74), ST VSAV (RQQQ) (SEQ ID NO: 75), or ST VSVNJ (QQQ) (SEQ ID NO: 73) bound to a recombinant fusion protein of SpyCatcher (SEQ ID NO: 30) fused with UCHT1 scFv (SEQ ID NOs: 45 and 46) as targeting molecule compared to the number of transduced Nalm6 cells. These results suggest that lentivirus pseudotyped with ST CJSV (RQQQ) (SEQ ID NO: 74), ST VSAV (RQQQ) (SEQ) ID NO: 75), or ST VSVNJ (QQQ) (SEQ ID NO: 73) bound to SpyCatcher with a targeting molecule have low background infectivity compared to wild type VSIV-G (SEQ ID NO: 21) and specifically bind cells expressing surface proteins that the targeting molecule can bind. This suggests that this system is versatile in displaying a variety of targeting molecules on the lentivirus surface pseudotyped with alternate viral glycoproteins. This suggests the potential of this method for production of virions capable of delivering genetic cargo specifically to target cells in an in vivo setting.

### Example 10

This example demonstrates the serum stability of lentiviruses pseudotyped with SpyTag VSIV-G bound to a SpyCatcher.

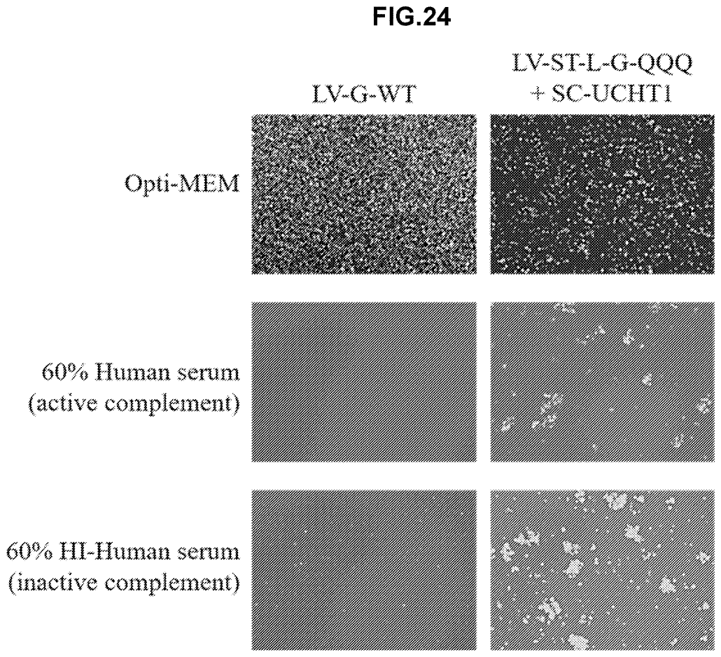

In order to test the efficacy of lentivirus pseudotyped with a recombinant fusion protein of SpyTag fused to receptor blinded rhabdoviral G glycoprotein bound to a recombinant fusion protein of SpyCatcher fused to a targeting molecule in vivo, experiments were performed to determine the serum stability of these lentiviruses. Jurkat cells were cultured in media containing Opti-MEM™, 60% complement active human serum, or 60% heat inactivated (HI) human serum and transduced with 20 μL of lentivirus containing a GFP-expression cassette and pseudotyped with either VSIV-G glycoprotein (LV-G-WT) (SEQ ID NO: 21) or a recombinant fusion protein of first generation SpyTag fused to VSIV-G-QQQ (SEQ ID NO: 66) bound to a recombinant fusion protein of first generation SpyCatcher fused to UCHT1 scFv (SEQ ID NO: 67) (LV-ST-L-G-QQQ+SC-UCHT1). 48 hours post-transduction fluorescent micrographs were captured by Nikon microscope at 10× objective lens (FIG. 24). These micrographs showed that lentiviruses pseudotyped with VSIV-G alone were inactivated by both complement active human serum and HI human serum, however the lentiviruses pseudotyped with the recombinant fusion proteins of SpyTag fused to VSIV-G-QQQ (SEQ ID NO: 66) bound to SpyCatcher fused to UCHT1 scFv (SEQ ID NO: 67) showed resistance to serum inactivation by both complement active human serum and HI human serum. These results suggest that lentiviruses pseudotyped with a recombinant protein of a SpyTag fused to a receptor blinded rhabdoviral G glycoprotein bound to a recombinant fusion protein of a SpyCatcher fused to a targeting molecule would be useful for specific cell types, such as T cells, in vivo as they demonstrate resistance to serum inactivation.

Lengthy table referenced here
US20260285912A1-20260924-T00001
Please refer to the end of the specification for access instructions.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

LENGTHY TABLES
The patent application contains a lengthy table section. A copy of the table is available in electronic form from the USPTO web site (https://seqdata.uspto.gov/docdetail?docId=US20260285912A1). An electronic copy of the table will also be available from the USPTO upon request and payment of the fee set forth in 37 CFR 1.19(b)(3).

1. A recombinant fusion protein comprising (a) a rhabdoviral G glycoprotein or a functional fragment or derivative thereof and (b) a polypeptide (polypeptide tag) that is capable of forming a covalent bond with an amino acid residue in a second recombinant fusion protein, wherein the polypeptide tag is N-terminal to the rhabdoviral G glycoprotein, and wherein the second recombinant fusion protein comprises a polypeptide (polypeptide catcher) that is capable of forming a covalent bond with an amino acid residue in the polypeptide tag.

2. The recombinant fusion protein of claim 1, polypeptide tag is SpyTag (SEQ ID NO: 1), SpyTag002 (SEQ ID NO: 2), SpyTag003 (SEQ ID NO: 3), Isopeptag (SEQ ID NO: 4), or SnoopTag (SEQ ID NO: 5).

3. The recombinant fusion protein of claim 2, wherein the polypeptide tag is SpyTag (SEQ ID NO: 1).

4. The recombinant fusion protein of any one of claims 1-3, wherein the fusion protein comprises a linker between the polypeptide tag and the rhabdoviral G glycoprotein or functional fragment or derivative thereof.

5. The recombinant fusion protein of claim 4, wherein the linker is flexible.

6. The recombinant fusion protein of claim 5, wherein the linker is
(SEQ ID NO: 130)
AAASGGSGGGGSGGGGSGP,
(SEQ ID NO: 131)
AAASGGSGGGGSGGGGS,
(SEQ ID NO: 132)
GGGGSGGGGSGGGGSGGGGS,
(SEQ ID NO: 36)
GGGGSGGGGSGGGGS,
(SEQ ID NO: 133)
GGGGSGGGGS,
(SEQ ID NO: 134)
GGGGS,
(SEQ ID NO: 135)
GGGGGGGG,
(SEQ ID NO: 136)
GGGGGG,
(SEQ ID NO: 137)
GSAGSAAGSGEF,
and
(SEQ ID NO: 138)
VPGVGVPGVG.

7. The recombinant fusion protein of claim 4, wherein the linker is rigid.

8. The recombinant fusion protein of claim 7, wherein the linker is
(SEQ ID NO: 139)
PAPAP,
(SEQ ID NO: 140)
EAAAKEAAAKEAAAK,
(SEQ ID NO: 141)
EAAAKEAAAK,
(SEQ ID NO: 142)
EAAAK,
(SEQ ID NO: 143)
AEAAAKEAAAKEAAAKEAAAKALEAEAAAKEAAAKEAAAKEAAAKA,
(SEQ ID NO: 144)
AEAAAKEAAAKA,
(SEQ ID NO: 145)
ESKYGPPCPPCP,
(SEQ ID NO: 146)
CPPCPAPELLGGPSVF,
and
alanine-proline (AP) repeated for a total of 10 to 34 amino acids (SEQ ID NO: 147).

9. The recombinant fusion protein of any one of claims 1-8, wherein the rhabdoviral G glycoprotein or functional fragment or derivative thereof is of a Flanders virus glycoprotein (FLAV-G) (SEQ ID NO: 13), a Chandipura virus glycoprotein (CHPV-G) (SEQ ID NO: 14), a Perinet virus glycoprotein (PERV-G) (SEQ ID NO: 15), a Piry virus glycoprotein (PIRYV-G) (SEQ ID NO: 16), a Fukuoka virus glycoprotein (FUKV-G) (SEQ ID NO: 17), a Joinjakaka virus glycoprotein (JOIV-G) (SEQ ID NO: 18), a Kumasi virus glycoprotein (KRV-G) (SEQ ID NO: 19), a Keuraliba virus glycoprotein (KEUV-G) (SEQ ID NO: 20), an Isfahan glycoprotein (ISFV-G), a Jurona glycoprotein (JURV-G), a Mediterranean Bat glycoprotein (MBV-G), a Malpais Spring glycoprotein (MSPV-G), a Radi glycoprotein (RADV-G), a Rhinolophus affinis-G, a Yug Bugdanavoc glycoprotein (YBV-G), a Yinshui Bat glycoprotein (YSBV-G), a Kimberley glycoprotein (KIMV-G), a Kanyawara glycoprotein (KYAV-G), a La Joya glycoprotein (LJV-G), a Mosquiero glycoprotein (MQOV-G), a Parry Creek glycoprotein (PCV-G), a Bas Congo glycoprotein (BASV-G), a Bovine Ephemeral fever glycoprotein (BEFV-G), a Curionopolis glycoprotein (CURV-G), a Drosophila melanogaster sigmavirus glycoprotein (DMelSV-G), a Niakha glycoprotein (NIAV-G), a Puerto almandras glycoprotein (PTAMV-G), or a Tupaia rhabdovirus (TUPTV-G).

10. The recombinant fusion protein of any one of claims 1-8, wherein the rhabdoviral G glycoprotein or functional fragment or derivative thereof is of Vesiculovirus glycoprotein or a functional fragment or derivative thereof.

11. The recombinant fusion protein of any one of claims 1-8, wherein the rhabdoviral G glycoprotein or functional fragment or derivative thereof is of Vesiculovirus indiana, Vesiculovirus newjersey, Vesiculovirus carajas, or Vesiculovirus alagoas.

12. The recombinant fusion protein of claim 11, wherein the rhabdoviral G glycoprotein or functional fragment or derivative thereof is of Vesiculovirus indiana (SEQ ID NO: 21).

13. The recombinant fusion protein of claim 11, wherein the rhabdoviral G glycoprotein or functional fragment or derivative thereof is of Vesiculovirus newjersey (SEQ ID NO: 22).

14. The recombinant fusion protein of any one of claims 1-13, wherein the rhabdoviral G glycoprotein is substantially intact.

15. The recombinant fusion protein of any one of claims 1-13, wherein the rhabdoviral G glycoprotein is a functional fragment or derivative thereof.

16. The recombinant fusion protein of claim 15, wherein the cytoplasmic tail of the glycoprotein is truncated, deleted, or replaced with another sequence.

17. The recombinant fusion protein of any one of claims 1-16, wherein the rhabdoviral G glycoprotein is engineered to reduce or abolish its natural receptor binding specificity.

18. The recombinant fusion protein of claim 17, wherein the rhabdoviral G glycoprotein is engineered to have a mutation to reduce or abolish its natural receptor binding specificity.

19. The recombinant fusion protein of claim 18, wherein the rhabdoviral G glycoprotein comprises a mutation at one or more positions corresponding to H8, K47, Y209, and K354 on the Vesiculovirus indiana glycoprotein (SEQ ID NO: 21).

20. The recombinant fusion protein of claim 18 or 19, wherein the mutation is a substitution.

21. The recombinant fusion protein of claim 20, wherein the substitution is with a Q.

22. The recombinant fusion protein of claim 20 or 21, wherein the mutation is a substitution at three or more positions corresponding to H8, K47, Y209, and K354 on the Vesiculovirus indiana glycoprotein (SEQ ID NO: 21).

23. The recombinant fusion protein of claim 18 or 19, wherein the mutation is a deletion.

24. The recombinant fusion protein of claim 23, wherein the mutation is a single deletion at K47.

25. The recombinant fusion protein of any one of claims 1-24, wherein the polypeptide tag is covalently bonded to the polypeptide catcher.

26. The recombinant fusion protein of claim 25, wherein the formation of the covalent bond is spontaneous.

27. The recombinant fusion protein of claim 25 or 26, wherein the covalent bond is an isopeptide bond.

28. The recombinant fusion protein of any one of claims 25-27, wherein the polypeptide tag and the polypeptide catcher are the pair SpyTag and SpyCatcher (SEQ ID NO: 1 and SEQ ID NO: 30), SpyTag002 and SpyCatcher002 (SEQ ID NO: 2 and SEQ ID NO: 31), SpyTag003 and SpyCatcher003 (SEQ ID NO: 3 and SEQ ID NO: 32), SpyTag and KTag (SEQ ID NO: 1 and SEQ ID NO: 33), Isopeptag and pilin-C(SEQ ID NO: 4 and SEQ ID NO: 34), or SnoopTag and SnoopCatcher (SEQ ID NO: 5 and SEQ ID NO: 35).

29. The recombinant fusion protein of any one of claims 25-28, wherein the second recombinant fusion protein comprises a targeting molecule.

30. The recombinant fusion protein of claim 29, wherein the targeting molecule is a ligand.

31. The recombinant fusion protein of claim 30, wherein the ligand is epidermal growth factor (EGF), a mutant EGF (EFGm123) (SEQ ID NO: 59), stem cell factor (SCF) (SEQ ID NO: 52), thrombopoietin (TPO) (SEQ ID NO: 55), human hepatocyte growth factor (HGF) (SEQ ID NO: 56), or type 1 insulin-like growth factor (IGF1) (SEQ ID NO: 58).

32. The recombinant fusion protein of claim 29, wherein the targeting molecule is a single chain variable fragment (scFv).

33. The recombinant fusion protein of claim 32, wherein the scFv binds epidermal growth factor (EGF), human epidermal growth factor receptor 2 (Her2), cluster of differentiation 3 (CD3), or cluster of differentiation 117 (cKit), mucin-16 (MUC16), B cell maturation antigen (BCMA), or Nectin4.

34. The recombinant fusion protein of any one of claims 29-33, wherein the second recombinant fusion protein comprises a linker between the targeting molecule and the polypeptide catcher.

35. The recombinant fusion protein of claim 34, wherein the linker is flexible.

36. The recombinant fusion protein of claim 35, wherein the linker is
(SEQ ID NO: 130)
AAASGGSGGGGSGGGGSGP,
(SEQ ID NO: 131)
AAASGGSGGGGSGGGGS,
(SEQ ID NO: 132)
GGGGSGGGGSGGGGSGGGGS,
(SEQ ID NO: 36)
GGGGSGGGGSGGGGS,
(SEQ ID NO: 133)
GGGGSGGGGS,
(SEQ ID NO: 134)
GGGGS,
(SEQ ID NO: 135)
GGGGGGGG,
(SEQ ID NO: 136)
GGGGGG,
(SEQ ID NO: 137)
GSAGSAAGSGEF,
and
(SEQ ID NO: 138)
VPGVGVPGVG.

37. The recombinant fusion protein of claim 34, wherein the linker is rigid.

38. The recombinant fusion protein of claim 37, wherein the linker is
(SEQ ID NO: 139)
PAPAP,
(SEQ ID NO: 140)
EAAAKEAAAKEAAAK,
(SEQ ID NO: 141)
EAAAKEAAAK,
(SEQ ID NO: 142)
EAAAK,
(SEQ ID NO: 143)
AEAAAKEAAAKEAAAKEAAAKALEAEAAAKEAAAKEAAAKEAAAKA,
(SEQ ID NO: 144)
AEAAAKEAAAKA,
(SEQ ID NO: 145)
ESKYGPPCPPCP,
(SEQ ID NO: 146)
CPPCPAPELLGGPSVF,
and
alanine-proline (AP) repeated for a total of 10 to 34 amino acids (SEQ ID NO: 147).

39. The recombinant fusion protein of any one of claims 29-38, wherein the targeting molecule is N terminal to the polypeptide catcher.

40. The recombinant fusion protein of any one of claims 29-38, wherein the targeting molecule is C terminal to the polypeptide catcher.

41. The recombinant fusion protein of any one of claims 25-40, wherein the second recombinant fusion protein comprises a signal peptide.

42. The recombinant fusion protein of any one of claims 25-41, wherein the second recombinant fusion protein comprises a tag for western detection or purification.

43. The recombinant fusion protein of claim 42, wherein the tag is a tag for western detection.

44. The recombinant fusion protein of claim 42, wherein the tag is a tag for purification.

45. A recombinant protein system comprising (a) a recombinant fusion protein comprising (1) a rhabdoviral G glycoprotein or a functional fragment or derivative thereof and (2) a polypeptide (polypeptide tag) that is capable of forming a covalent bond with an amino acid residue in a second recombinant fusion protein, wherein the polypeptide tag is N-terminal to the rhabdoviral G glycoprotein, and (b) the second recombinant fusion protein, wherein the second recombinant fusion protein comprises a polypeptide (polypeptide catcher) that is capable of forming a covalent bond with an amino acid residue in the polypeptide tag.

46. The recombinant protein system of claim 45, wherein the polypeptide tag and the polypeptide catcher are the pair SpyTag and SpyCatcher (SEQ ID NO: 1 and SEQ ID NO: 30), SpyTag002 and SpyCatcher002 (SEQ ID NO: 2 and SEQ ID NO: 31), SpyTag003 and SpyCatcher003 (SEQ ID NO: 3 and SEQ ID NO: 32), SpyTag and KTag (SEQ ID NO: 1 and SEQ ID NO: 33), Isopeptag and pilin-C(SEQ ID NO: 4 and SEQ ID NO: 34), or SnoopTag and SnoopCatcher (SEQ ID NO: 5 and SEQ ID NO: 35).

47. The recombinant protein system of claim 46, wherein the polypeptide tag and the polypeptide catcher are the pair SpyTag003 and SpyCatcher003 (SEQ ID NO: 3 and SEQ ID NO: 32).

48. The recombinant protein system of any one of claims 45-47, wherein the recombinant fusion protein comprises a linker between the polypeptide tag and the rhabdoviral G glycoprotein or functional fragment or derivative thereof.

49. The recombinant protein system of claim 48, wherein the linker is flexible.

50. The recombinant protein system of claim 49, wherein the linker is
(SEQ ID NO: 130)
AAASGGSGGGGSGGGGSGP,
(SEQ ID NO: 131)
AAASGGSGGGGSGGGGS,
(SEQ ID NO: 132)
GGGGSGGGGSGGGGSGGGGS,
(SEQ ID NO: 36)
GGGGSGGGGSGGGGS,
(SEQ ID NO: 133)
GGGGSGGGGS,
(SEQ ID NO: 134)
GGGGS,
(SEQ ID NO: 135)
GGGGGGGG,
(SEQ ID NO: 136)
GGGGGG,
(SEQ ID NO: 137)
GSAGSAAGSGEF,
and
(SEQ ID NO: 138)
VPGVGVPGVG.

51. The recombinant protein system of claim 48, wherein the linker is rigid.

52. The recombinant protein system of claim 51, wherein the linker is
(SEQ ID NO: 139)
PAPAP,
(SEQ ID NO: 140)
EAAAKEAAAKEAAAK,
(SEQ ID NO: 141)
EAAAKEAAAK,
(SEQ ID NO: 142)
EAAAK,
(SEQ ID NO: 143)
AEAAAKEAAAKEAAAKEAAAKALEAEAAAKEAAAKEAAAKEAAAKA,
(SEQ ID NO: 144)
AEAAAKEAAAKA,
(SEQ ID NO: 145)
ESKYGPPCPPCP,
(SEQ ID NO: 146)
CPPCPAPELLGGPSVF,
and
alanine-proline (AP) repeated for a total of 10 to 34 amino acids (SEQ ID NO: 147).

53. The recombinant protein system of any one of claims 45-52, wherein the rhabdoviral G glycoprotein or functional fragment or derivative thereof is of a Flanders virus glycoprotein (FLAV-G) (SEQ ID NO: 13), a Chandipura virus glycoprotein (CHPV-G) (SEQ ID NO: 14), a Perinet virus glycoprotein (PERV-G) (SEQ ID NO: 15), a Piry virus glycoprotein (PIRYV-G) (SEQ ID NO: 16), a Fukuoka virus glycoprotein (FUKV-G) (SEQ ID NO: 17), a Joinjakaka virus glycoprotein (JOIV-G) (SEQ ID NO: 18), a Kumasi virus glycoprotein (KRV-G) (SEQ ID NO: 19), a Keuraliba virus glycoprotein (KEUV-G) (SEQ ID NO: 20), an Isfahan glycoprotein (ISFV-G), a Jurona glycoprotein (JURV-G), a Mediterranean Bat glycoprotein (MBV-G), a Malpais Spring glycoprotein (MSPV-G), a Radi glycoprotein (RADV-G), a Rhinolophus affinis-G, a Yug Bugdanavoc glycoprotein (YBV-G), a Yinshui Bat glycoprotein (YSBV-G), a Kimberley glycoprotein (KIMV-G), a Kanyawara glycoprotein (KYAV-G), a La Joya glycoprotein (LJV-G), a Mosquiero glycoprotein (MQOV-G), a Parry Creek glycoprotein (PCV-G), a Bas Congo glycoprotein (BASV-G), a Bovine Ephemeral fever glycoprotein (BEFV-G), a Curionopolis glycoprotein (CURV-G), a Drosophila melanogaster sigmavirus glycoprotein (DMelSV-G), a Niakha glycoprotein (NIAV-G), a Puerto almandras glycoprotein (PTAMV-G), or a Tupaia rhabdovirus (TUPTV-G).

54. The recombinant protein system of any one of claims 45-52, wherein the rhabdoviral G glycoprotein or functional fragment or derivative thereof is of a Vesiculovirus glycoprotein or a functional fragment or derivative thereof.

55. The recombinant protein system of any one of claims 45-52, wherein the rhabdoviral G glycoprotein or functional fragment or derivative thereof is of Vesiculovirus indiana, Vesiculovirus newjersey, Vesiculovirus carajas, or Vesiculovirus alagoas.

56. The recombinant protein system of claim 55, wherein the rhabdoviral G glycoprotein or functional fragment or derivative thereof is of Vesiculovirus indiana (SEQ ID NO: 21).

57. The recombinant protein system of claim 55, wherein the rhabdoviral G glycoprotein or functional fragment or derivative thereof is of Vesiculovirus newjersey (SEQ ID NO: 22).

58. The recombinant protein system of any one of claims 45-57, wherein the rhabdoviral G glycoprotein is substantially intact.

59. The recombinant protein system of any one of claims 45-57, wherein the rhabdoviral G glycoprotein is a functional fragment or derivative thereof.

60. The recombinant protein system of claim 59, wherein the cytoplasmic tail of the glycoprotein is truncated, deleted, or replaced with another sequence.

61. The recombinant protein system of any one of claims 45-60, wherein the rhabdoviral G glycoprotein is engineered to reduce or abolish its natural receptor binding specificity.

62. The recombinant protein system of claim 61, wherein the rhabdoviral G glycoprotein is engineered to have a mutation to reduce or abolish its natural receptor binding specificity.

63. The recombinant protein system of claim 62, wherein the rhabdoviral G glycoprotein comprises a mutation at one or more positions corresponding to H8, K47, Y209, and K354 on the Vesiculovirus indiana glycoprotein (SEQ ID NO: 21)

64. The recombinant protein system of claim 62 or 63, wherein the mutation is a substitution.

65. The recombinant protein system of claim 64, wherein the substitution is with a Q.

66. The recombinant protein system of claim 64 or 65, wherein the mutation is a substitution at three or more positions corresponding to H8, K47, Y209, and K354 on the Vesiculovirus indiana glycoprotein (SEQ ID NO: 21)

67. The recombinant protein system of claim 62 or 63, wherein the mutation is a deletion.

68. The recombinant protein system of claim 67, wherein the mutation is a single deletion at K47.

69. The recombinant protein system of any one of claims 45-68, wherein the second recombinant fusion protein comprises a targeting molecule.

70. The recombinant protein system of claim 69, wherein the targeting molecule is a ligand.

71. The recombinant protein system of claim 70, wherein the ligand is epidermal growth factor (EGF), a mutant EGF (EFGm123) (SEQ ID NO: 59), stem cell factor (SCF) (SEQ ID NO: 52), thrombopoietin (TPO) (SEQ ID NO: 55), human hepatocyte growth factor (HGF) (SEQ ID NO: 56), or type 1 insulin-like growth factor (IGF1) (SEQ ID NO: 58).

72. The recombinant protein system of claim 69, wherein the targeting molecule is a single chain variable fragment (scFv).

73. The recombinant protein system of claim 72, wherein the scFv binds epidermal growth factor (EGF), human epidermal growth factor receptor 2 (Her2), cluster of differentiation 3 (CD3), or cluster of differentiation 117 (cKit), mucin-16 (MUC16), B cell maturation antigen (BCMA), or Nectin4.

74. The recombinant protein system of any one of claims 69-73, wherein the second recombinant fusion protein comprises a linker between the targeting molecule and the polypeptide catcher.

75. The recombinant protein system of claim 74, wherein the linker is flexible.

76. The recombinant protein system of claim 75, wherein the linker is
(SEQ ID NO: 130)
AAASGGSGGGGSGGGGSGP,
(SEQ ID NO: 131)
AAASGGSGGGGSGGGGS,
(SEQ ID NO: 132)
GGGGSGGGGSGGGGSGGGGS,
(SEQ ID NO: 36)
GGGGSGGGGSGGGGS,
(SEQ ID NO: 133)
GGGGSGGGGS,
(SEQ ID NO: 134)
GGGGS,
(SEQ ID NO: 135)
GGGGGGGG,
(SEQ ID NO: 136)
GGGGGG,
(SEQ ID NO: 137)
GSAGSAAGSGEF,
and
(SEQ ID NO: 138)
VPGVGVPGVG.

77. The recombinant protein system of claim 74, wherein the linker is rigid.

78. The recombinant protein system of claim 77, wherein the linker is
(SEQ ID NO: 139)
PAPAP,
(SEQ ID NO: 140)
EAAAKEAAAKEAAAK,
(SEQ ID NO: 141)
EAAAKEAAAK,
(SEQ ID NO: 142)
EAAAK,
(SEQ ID NO: 143)
AEAAAKEAAAKEAAAKEAAAKALEAEAAAKEAAAKEAAAKEAAAKA,
(SEQ ID NO: 144)
AEAAAKEAAAKA,
(SEQ ID NO: 145)
ESKYGPPCPPCP,
(SEQ ID NO: 146)
CPPCPAPELLGGPSVF,
and
alanine-proline (AP) repeated for a total of 10 to 34 amino acids (SEQ ID NO: 147).

79. The recombinant protein system of any one of claims 69-78, wherein the targeting molecule is N terminal to the polypeptide catcher.

80. The recombinant protein system of any one of claims 69-78, wherein the targeting molecule is C terminal to the polypeptide catcher.

81. The recombinant protein system of any one of claims 45-80, wherein the second recombinant fusion protein comprises a signal peptide.

82. The recombinant protein system of any one of claims 45-80, wherein the second recombinant fusion protein comprises a tag for western detection or purification.

83. The recombinant protein system of claim 82, wherein the tag is a tag for western detection.

84. The recombinant protein system of claim 82, wherein the tag is a tag for purification.

85. A membraned vesicle comprising the recombinant fusion protein of any one of claims 1-44 or the recombinant protein system of any one of claims 45-84.

86. The membraned vesicle of claim 85, wherein the vesicle is a gesicle or an exosome.

87. The membraned vesicle of claim 85 or 86, wherein the membraned vesicle comprises a mixed trimer, wherein the mixed trimer comprises (a) at least one recombinant fusion protein, wherein the polypeptide tag of the recombinant fusion protein is covalently bound to the polypeptide catcher of the second recombinant fusion protein, and (b) at least one (i) recombinant fusion protein, wherein the polypeptide tag of the recombinant fusion protein is not covalently bound to a polypeptide catcher or (ii) rhabdoviral G glycoprotein or functional fragment or derivative thereof that is not within a recombinant fusion protein.

88. The membraned vesicle of claim 87, wherein the membraned vesicle comprises an unmixed trimer, wherein the unmixed trimer comprises only rhabdoviral G glycoprotein or a functional fragment or derivative thereof that is not within a recombinant fusion protein.

89. The membraned vesicle of claim 88, wherein the membraned vesicle comprises the maximum number of the recombinant fusion proteins, wherein the polypeptide tag of the recombinant fusion protein is covalently bound to the polypeptide catcher of the second recombinant fusion protein, possible to be accommodated within the membrane of the membraned vesicle.

90. An enveloped viral particle comprising the recombinant fusion protein of any one of claims 1-44 or the recombinant protein system of any one of claims 45-84.

91. The enveloped viral particle of claim 90, wherein the enveloped viral particle comprises a mixed trimer, wherein the mixed trimer comprises (a) at least one recombinant fusion protein, wherein the polypeptide tag of the recombinant fusion protein is covalently bound to the polypeptide catcher of the second recombinant fusion protein, and (b) at least one (i) recombinant fusion protein, wherein the polypeptide tag of the recombinant fusion protein is not covalently bound to a polypeptide catcher or (ii) rhabdoviral G glycoprotein or functional fragment or derivative thereof that is not within a recombinant fusion protein.

92. The enveloped viral particle of claim 91, wherein the enveloped viral particle comprises an unmixed trimer, wherein the unmixed trimer comprises only rhabdoviral G glycoprotein or a functional fragment or derivative thereof that is not within a recombinant fusion protein.

93. The enveloped viral particle of claim 92, wherein the enveloped viral particle comprises the maximum number of the recombinant fusion proteins possible to be accommodated within the membrane of the enveloped viral particle.

94. A recombinant viral vector comprising a nucleotide encapsulated by the membraned vesicle of any one of claims 85-89 or the enveloped viral particle of any one of claims 90-93.

95. A composition comprising a pharmaceutically acceptable carrier and the membraned vesicle of any one of claims 85-89, the enveloped viral particle of any one of claims 90-93, or the recombinant viral vector of claim 94.

96. A method of delivering a payload to a target cell, the method comprising contacting the target cell with the membraned vesicle of any one of claims 85-89, the enveloped viral particle of any one of claims 90-93, the recombinant viral vector of claim 94, or the composition of claim 95.

97. The method of claim 96, wherein the target cell in in vitro or ex vivo.

98. The method of claim 96, wherein the target cell in in vivo.

99. The method of any one of claims 96-98, wherein the payload is a nucleotide sequence encoding a chimeric antigen receptor (CAR) comprising an antigen binding domain, a transmembrane domain, and an intracellular signaling domain.

100. A retroviral vector expression system comprising one or more nucleotide sequences encoding the recombinant fusion protein of any one of claims 1-44 or the recombinant protein system of any one of claims 45-84.

101. The retroviral vector expression system of claim 100, wherein the retroviral vector expression system comprises a vector construct and helper constructs that are each on separate plasmids.

102. The retroviral vector expression system of claim 100 or 101, wherein the retroviral vector expression system is a lentirival vector expression system.

103. The retroviral vector expression system of any one of claims 100-102, comprising a nucleotide sequence encoding a chimeric antigen receptor (CAR) comprising an antigen binding domain, a transmembrane domain, and an intracellular signaling domain.

104. A method of making a membraned vesicle, an enveloped viral particle, or a recombinant viral vector, the method comprising:
a) transfecting or transducing a packaging host cell with the retroviral vector expression system of any one of claims 100-103; and
b) recovering the membraned vesicle, enveloped viral particle, or recombinant viral vector produced by the transfected or transduced packaging host cell.

105. A method of making a membraned vesicle, an enveloped viral particle, or a recombinant viral vector, the method comprising:
a) transfecting or transducing a first host cell with a first vector expression system comprising one or more nucleotide sequences encoding the recombinant fusion protein of any one of claims 1-24, wherein the first host cell is a packaging host cell;
b) transfecting or transducing a second host cell with a second vector expression system comprising one or more nucleotide sequences encoding the second recombinant fusion protein;
c) collecting supernatant from the transfected or transduced second host cell;
d) administering the supernatant to the transfected or transduced first host cell; and
e) recovering the membraned vesicle, enveloped viral particle, or recombinant viral vector produced by the transfected or transduced first host cell after administration of supernatant produced by the transfected or transduced second host cell.

106. The method of claim 105, wherein the polypeptide tag and the polypeptide catcher are the pair SpyTag and SpyCatcher (SEQ ID NO: 1 and SEQ ID NO: 30), SpyTag002 and SpyCatcher002 (SEQ ID NO: 2 and SEQ ID NO: 31), SpyTag003 and SpyCatcher003 (SEQ ID NO: 3 and SEQ ID NO: 32), SpyTag and KTag (SEQ ID NO: 1 and SEQ ID NO: 33), Isopeptag and pilin-C(SEQ ID NO: 4 and SEQ ID NO: 34), or SnoopTag and SnoopCatcher (SEQ ID NO: 5 and SEQ ID NO: 35).

107. The method of claim 105 or 106, wherein the second recombinant fusion protein comprises a targeting molecule.

108. The method of claim 107, wherein the targeting molecule is a ligand.

109. The method of claim 108, wherein the ligand is epidermal growth factor (EGF), a mutant EGF (EFGm123) (SEQ ID NO: 59), stem cell factor (SCF) (SEQ ID NO: 52), thrombopoietin (TPO) (SEQ ID NO: 55), human hepatocyte growth factor (HGF) (SEQ ID NO: 56), or type 1 insulin-like growth factor (IGF1) (SEQ ID NO: 58).

110. The method of claim 107, wherein the targeting molecule is a single chain variable fragment (scFv).

111. The method of claim 110, wherein the scFv binds epidermal growth factor (EGF), human epidermal growth factor receptor 2 (Her2), cluster of differentiation 3 (CD3), or cluster of differentiation 117 (cKit), mucin-16 (MUC16), B cell maturation antigen (BCMA), or Nectin4.

112. The method of any one of claims 107-111, wherein the second recombinant fusion protein comprises a linker between the targeting molecule and the polypeptide catcher.

113. The method of claim 112, wherein the linker is flexible.

114. The method of claim 113, wherein the linker is
(SEQ ID NO: 130)
AAASGGSGGGGSGGGGSGP,
(SEQ ID NO: 131)
AAASGGSGGGGSGGGGS,
(SEQ ID NO: 132)
GGGGSGGGGSGGGGSGGGGS,
(SEQ ID NO: 36)
GGGGSGGGGSGGGGS,
(SEQ ID NO: 133)
GGGGSGGGGS,
(SEQ ID NO: 134)
GGGGS,
(SEQ ID NO: 135)
GGGGGGGG,
(SEQ ID NO: 136)
GGGGGG,
(SEQ ID NO: 137)
GSAGSAAGSGEF,
and
(SEQ ID NO: 138)
VPGVGVPGVG.

115. The method of claim 112, wherein the linker is rigid.

116. The method of claim 115, wherein the linker is
(SEQ ID NO: 139)
PAPAP,
(SEQ ID NO: 140)
EAAAKEAAAKEAAAK,
(SEQ ID NO: 141)
EAAAKEAAAK,
(SEQ ID NO: 142)
EAAAK,
(SEQ ID NO: 143)
AEAAAKEAAAKEAAAKEAAAKALEAEAAAKEAAAKEAAAKEAAAKA,
(SEQ ID NO: 144)
AEAAAKEAAAKA,
(SEQ ID NO: 145)
ESKYGPPCPPCP,
(SEQ ID NO: 146)
CPPCPAPELLGGPSVF,
and
alanine-proline (AP) repeated for a total of 10 to 34 amino acids (SEQ ID NO: 147).

117. The method of any one of claims 107-116, wherein the targeting molecule is N terminal to the polypeptide catcher.

118. The method of any one of claims 107-116, wherein the targeting molecule is C terminal to the polypeptide catcher.

119. The method of any one of claims 105-118, wherein the second recombinant fusion protein comprises a signal peptide.

120. The method of any one of claims 105-119, wherein the second recombinant fusion protein comprises a tag for western detection or purification.

121. The method of claim 120, wherein the tag is a tag for western detection.

122. The method of claim 120, wherein the tag is a tag for purification.

123. The method of claim any one of claims 105-122, wherein the packaging host cell is HEK 293T or a 293 cell suspension line.

124. A plasmid comprising one or more nucleotide sequences encoding the recombinant fusion protein of any one of claims 1-44 or the recombinant protein system of any one of claims 45-84.

125. The composition of claim 95 or the retroviral vector expression system of any one of claims 100-103 for use in treating a disease in a mammal.

126. The method of claim 125, wherein the mammal is a human.

127. The method of claim 125 or 126, wherein the disease is a genetic disease.

128. The method of claim 125 or 126, wherein the disease is a cancer.

129. A method of making a mixed rhabdoviral G glycoprotein trimer, the method comprising:
a. transfecting or transducing a packaging host cell with the retroviral vector expression system of any one of claims 100-103; and
b. recovering the mixed rhabdoviral G glycoprotein trimer.

130. A method of making a mixed rhabdoviral G glycoprotein trimer, the method comprising:
a. transfecting or transducing a first host cell with a first vector expression system comprising one or more nucleotide sequences encoding the recombinant fusion protein of any one of claims 1-24, wherein the first host cell is a packaging host cell;
b. transfecting or transducing a second host cell with a second vector expression system comprising one or more nucleotide sequences encoding the second recombinant fusion protein;
c. contacting virus produced by the first host cell with second recombinant fusion protein produced by the second host cell; and
d. obtaining virus decorated with the second recombinant fusion protein,
wherein the virus comprises a mixed trimer, wherein the mixed trimer comprises (a) at least one recombinant fusion protein, wherein the polypeptide tag of the recombinant fusion protein is covalently bound to the polypeptide catcher of the second recombinant fusion protein, and (b) at least one (i) recombinant fusion protein, wherein the polypeptide tag of the recombinant fusion protein is not covalently bound to a polypeptide catcher or (ii) rhabdoviral G glycoprotein or a functional fragment or derivative thereof that is not within a recombinant fusion protein.

131. The method of claim 130, wherein the polypeptide tag and the polypeptide catcher are the pair SpyTag and SpyCatcher (SEQ ID NO: 1 and SEQ ID NO: 30), SpyTag002 and SpyCatcher002 (SEQ ID NO: 2 and SEQ ID NO: 31), SpyTag003 and SpyCatcher003 (SEQ ID NO: 3 and SEQ ID NO: 32), SpyTag and KTag (SEQ ID NO: 1 and SEQ ID NO: 33), Isopeptag and pilin-C(SEQ ID NO: 4 and SEQ ID NO: 34), or SnoopTag and SnoopCatcher (SEQ ID NO: 5 and SEQ ID NO: 35).

132. The method of claim 130 or 131, wherein the second recombinant fusion protein comprises a targeting molecule.

133. The method of claim 132, wherein the targeting molecule is a ligand.

134. The method of claim 133, wherein the ligand is epidermal growth factor (EGF), a mutant EGF (EFGm123) (SEQ ID NO: 59), stem cell factor (SCF) (SEQ ID NO: 52), thrombopoietin (TPO) (SEQ ID NO: 55), human hepatocyte growth factor (HGF) (SEQ ID NO: 56), or type 1 insulin-like growth factor (IGF1) (SEQ ID NO: 58).

135. The method of claim 132, wherein the targeting molecule is a single chain variable fragment (scFv).

136. The method of claim 135, wherein the scFv binds epidermal growth factor (EGF), human epidermal growth factor receptor 2 (Her2), cluster of differentiation 3 (CD3), or cluster of differentiation 117 (cKit), mucin-16 (MUC16), B cell maturation antigen (BCMA), or Nectin4.

137. The method of any one of claims 132-136, wherein the second recombinant fusion protein comprises a linker between the targeting molecule and the polypeptide catcher.

138. The method of claim 137, wherein the linker is flexible.

139. The method of claim 138, wherein the linker is
(SEQ ID NO: 130)
AAASGGSGGGGSGGGGSGP,
(SEQ ID NO: 131)
AAASGGSGGGGSGGGGS,
(SEQ ID NO: 132)
GGGGSGGGGSGGGGSGGGGS,
(SEQ ID NO: 36)
GGGGSGGGGSGGGGS,
(SEQ ID NO: 133)
GGGGSGGGGS,
(SEQ ID NO: 134)
GGGGS,
(SEQ ID NO: 135)
GGGGGGGG,
(SEQ ID NO: 136)
GGGGGG,
(SEQ ID NO: 137)
GSAGSAAGSGEF,
and
(SEQ ID NO: 138)
VPGVGVPGVG.

140. The method of claim 137, wherein the linker is rigid.

141. The method of claim 140, wherein the linker is
(SEQ ID NO: 139)
PAPAP,
(SEQ ID NO: 140)
EAAAKEAAAKEAAAK,
(SEQ ID NO: 141)
EAAAKEAAAK,
(SEQ ID NO: 142)
EAAAK,
(SEQ ID NO: 143)
AEAAAKEAAAKEAAAKEAAAKALEAEAAAKEAAAKEAAAKEAAAKA,
(SEQ ID NO: 144)
AEAAAKEAAAKA,
(SEQ ID NO: 145)
ESKYGPPCPPCP,
(SEQ ID NO: 146)
CPPCPAPELLGGPSVF,
and
alanine-proline (AP) repeated for a total of 10 to 34 amino acids (SEQ ID NO: 147).

142. The method of any one of claims 132-141, wherein the targeting molecule is N terminal to the polypeptide catcher.

143. The method of any one of claims 132-141, wherein the targeting molecule is C terminal to the polypeptide catcher.

144. The method of any one of claims 130-143, wherein the second recombinant fusion protein comprises a signal peptide.

145. The method of any one of claims 130-144, wherein the second recombinant fusion protein comprises a tag for western detection or purification.

146. The method of claim 145, wherein the tag is a tag for western detection.

147. The method of claim 145, wherein the tag is a tag for purification.

148. The method of claim any one of claims 130-147, wherein the packaging host cell is HEK 293T or a 293 cell suspension line.

149. A method of reducing inhibition of rhabdoviral G glycoprotein-mediated fusion in serum, wherein the inhibition is by complement inactivation or competitive binding with LDL or vLDL, the method comprising covalently bonding to a rhabdoviral G glycoprotein or functional fragment or derivative thereof a targeting molecule, and exposing the rhabdoviral G glycoprotein or functional fragment or derivative thereof with targeting molecule to serum, wherein complement inactivation or competitive binding with LDL or vLDL is inhibited.

150. The method of claim 149, wherein the targeting molecule is a ligand.

151. The method of claim 150, wherein the ligand is epidermal growth factor (EGF), a mutant EGF (EFGm123) (SEQ ID NO: 59), stem cell factor (SCF) (SEQ ID NO: 52), thrombopoietin (TPO) (SEQ ID NO: 55), human hepatocyte growth factor (HGF) (SEQ ID NO: 56), or type 1 insulin-like growth factor (IGF1) (SEQ ID NO: 58).

152. The method of claim 149, wherein the targeting molecule is a single chain variable fragment (scFv).

153. The method of claim 152, wherein the scFv binds epidermal growth factor (EGF), human epidermal growth factor receptor 2 (Her2), cluster of differentiation 3 (CD3), or cluster of differentiation 117 (cKit), mucin-16 (MUC16), B cell maturation antigen (BCMA), or Nectin4.

154. The method of any one of claims 149-153, wherein the second recombinant fusion protein comprises a linker between the targeting molecule and the polypeptide catcher.

155. The method of claim 154, wherein the linker is flexible.

156. The method of claim 155, wherein the linker is
(SEQ ID NO: 130)
AAASGGSGGGGSGGGGSGP,
(SEQ ID NO: 131)
AAASGGSGGGGSGGGGS,
(SEQ ID NO: 132)
GGGGSGGGGSGGGGSGGGGS,
(SEQ ID NO: 36)
GGGGSGGGGSGGGGS,
(SEQ ID NO: 133)
GGGGSGGGGS,
(SEQ ID NO: 134)
GGGGS,
(SEQ ID NO: 135)
GGGGGGGG,
(SEQ ID NO: 136)
GGGGGG,
(SEQ ID NO: 137)
GSAGSAAGSGEF,
and
(SEQ ID NO: 138)
VPGVGVPGVG.

157. The method of claim 154, wherein the linker is rigid.

158. The method of claim 157, wherein the linker is
(SEQ ID NO: 139)
PAPAP,
(SEQ ID NO: 140)
EAAAKEAAAKEAAAK,
(SEQ ID NO: 141)
EAAAKEAAAK,
(SEQ ID NO: 142)
EAAAK,
(SEQ ID NO: 143)
AEAAAKEAAAKEAAAKEAAAKALEAEAAAKEAAAKEAAAKEAAAKA,
(SEQ ID NO: 144)
AEAAAKEAAAKA,
(SEQ ID NO: 145)
ESKYGPPCPPCP,
(SEQ ID NO: 146)
CPPCPAPELLGGPSVF,
and
alanine-proline (AP) repeated for a total of 10 to 34 amino acids (SEQ ID NO: 147).

159. A method of reducing inhibition of rhabdoviral G glycoprotein-mediated fusion in serum, wherein the inhibition is by complement inactivation or competitive binding with LDL or vLDL, the method comprising producing the rhabdoviral G glycoprotein or functional fragment or derivative thereof as the rhabdoviral G glycoprotein or functional fragment or derivative thereof within the recombinant fusion protein of any one of claims 25-44, and exposing the rhabdoviral G glycoprotein or functional fragment or derivative thereof to serum, wherein complement inactivation or competitive binding with LDL or vLDL is inhibited.
